(19) United States
(12) Patent Application Publication
(10) Pub. No.: US 2026/0097097 A1
(43) Pub. Date: Apr. 9, 2026

(54) FORMULATIONS COMPRISING ACTRIIA POLYPEPTIDE VARIANTS

(71) Applicant: MERCK SHARP & DOHME LLC, Rahway, NJ (US)

(72) Inventors: Vikram Sadineni, Rahway, NJ (US); Meena Nikhil Bhiwankar, Rahway, NJ (US); Xiaoqing Liu, Rahway, NJ (US)

(21) Appl. No.: 19/321,957

(22) Filed: Sep. 8, 2025

Related U.S. Application Data
(63) Continuation of application No. PCT/US2024/013378, filed on Jan. 29, 2024.
(60) Provisional application No. 63/451,198, filed on Mar. 9, 2023.

(51) Int. Cl. A61K 38/17; A61K 9/19; A61K 47/12; A61K 47/22; A61K 47/26

(52) U.S. Cl. CPC A61K 38/179; A61K 9/19; A61K 47/12; A61K 47/22; A61K 47/26

(57) ABSTRACT

In certain aspects, the present disclosure provides lyophilized pharmaceutical formulations comprising a recombinant fusion protein comprising an extracellular domain (ECD) of human activin receptor type-IIA (ActRIIa) proteins or derivatives thereof linked to a constant domain of an immunoglobulin, such as human IgG1 Fc domain and one or more pharmaceutical additives and/or excipients.

### CROSS-REFERENCE TO RELATED APPLICATION

This application is a continuation of International Patent Application No. PCT/US2024/013378, filed Jan. 29, 2024, which claims the benefit of U.S. Provisional Application No. 63/451,198, filed Mar. 9, 2023, the content of which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Sep. 8, 2025, is named Merck_220265_Sequence_Listing.xml and is 37,246 bytes in size.

### FIELD

Described herein are lyophilized pharmaceutical formulations comprising a recombinant fusion protein comprising an extracellular domain (ECD) of human activin receptor type-IIA (ActRIIa) protein or derivatives thereof linked to a constant domain of an immunoglobulin, such as human IgG1 Fc domain and one or more pharmaceutical additives and/or excipients as defined herein.

### BACKGROUND

Pulmonary hypertension (PH) is a disease characterized by high blood pressure in lung vasculature, including pulmonary arteries, pulmonary veins, and pulmonary capillaries. In general, PH is defined as a mean pulmonary arterial (PA) pressure ≥25 mm Hg at rest or ≥30 mm Hg with exercise (Hill et al., Respiratory Care 54(7): 958-68 (2009)). The main PH symptom is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches (Simonneau et al., JACC 54(1):S44-54 (2009)). Clinical classification of PH was first proposed in 1973, and a recent updated clinical classification was endorsed by the World Health Organization (WHO) in 2008. According to the updated PH clinical classification, there are five main groups of PH: (1) pulmonary arterial hypertension (PAH), characterized by a PA wedge pressure ≤15 mm Hg; (2) PH owing to a left heart disease (also known as pulmonary venous hypertension or congestive heart failure), (3) PH characterized by a PA wedge pressure >15 mm Hg; (4) PH owing to lung diseases and/or hypoxia; chronic thromboemboli PH; and (5) PH with unclear or multifactorial etiologies (Simonneau et al., JACC 54(1):S44-54(2009); Hill et al., Respiratory Care 54(7):958-68(2009)). PAH is further classified into idiopathic PAH (IPAH), a sporadic disease in which there is neither a family history of PAH nor an identified risk factor; heritable PAH; PAH induced by drugs and toxins; PAH associated with connective tissue diseases, HIV infection, portal hypertension, congenital heart diseases, schistosomiasis, and chronic hemolytic anemia; and persistent PH of new borns (Simonneau et al., JACC 54(1):S44-54 (2009)). Diagnosis of various types of PH requires a series of tests.

In general, PH treatment depends on the cause or classification of the PH. Where PH is caused by a known medicine or medical condition, it is known as a secondary PH, and its treatment is usually directed at the underlying disease. Treatment of pulmonary venous hypertension generally involves optimizing left ventricular function by administering diuretics, beta blockers, and ACE inhibitors, or repairing or replacing a mitral valve or aortic valve. PAH therapies include pulmonary vasodilators, digoxin, diuretics, anticoagulants, and oxygen therapy. Pulmonary vasodilators target different pathways, including prostacyclin pathway (e.g., prostacyclins, including intravenous epoprostenol, subcutaneous or intravenous treprostinil, and inhaled iloprost), nitric oxide pathway (e.g., phosphodiesterase-5 inhibitors, including sildenafil and tadalafil), and endotheline-1 pathway (e.g., endothelin receptor antagonists, including oral bosentan and oral ambrisentan) (Humbert, M. Am. J. Respir. Crit. Care Med. 179:650-6 (2009); Hill et al., Respiratory Care 54(7): 958-68 (2009)). However, current therapies provide no cure for PH, and they do not directly treat the underling vascular remodeling and muscularization of blood vessels observed in many PH patients.

Thus, it is an object of the present disclosure to provide pharmaceutical formulations comprising an ActRIIa fusion protein and corresponding methods for treating, preventing, or reducing the progression rate and/or severity of PH, particular treating, preventing or reducing the progression rate and/or severity of one or more PH-associated complications.

### SUMMARY

Provided herein are pharmaceutical formulations comprising a recombinant fusion protein comprising an extracellular domain (ECD) of human activin receptor type-IIA (ActRIIa) or a variant thereof linked to a constant domain of an immunoglobulin, such as a human IgG1 Fc domain, and one or more pharmaceutical additives and/or excipients, wherein the formulation is lyophilized.

In some embodiments, the ActRIIa protein comprises an amino acid sequence that is at least 70% (e.g., at least 70%, 75%, 80%, 85%, 86%, 87%, 88%, 89%, 90%, 91%, 92%, 93%, 94%, 95%, 96%, 97%, 98%, 99%, or 100%) identical to an amino acid sequence that begins at any one of amino acids 21, 22, 23, 24, 25, 26, 27, 28, 29, or 30 of SEQ ID NO: 9 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 9. In some embodiments, the ActRIIa protein comprises an amino acid sequence that is at least 70% (e.g., at least 70%, 75%, 80%, 85%, 86%, 87%, 88%, 89%, 90%, 91%, 92%, 93%, 94%, 95%, 96%, 97%, 98%, 99%, or 100%) identical to the amino acid sequence of SEQ ID NO: 10. In some embodiments, the ActRIIa protein comprises an amino acid sequence that is at least 70% (e.g., at least 70%, 75%, 80%, 85%, 86%, 87%, 88%, 89%, 90%, 91%, 92%, 93%, 94%, 95%, 96%, 97%, 98%, 99%, or 100%) identical to the amino acid sequence of SEQ ID NO: 11. In some embodiments, the ActRIIa protein is a fusion protein comprising an ActRIIa extracellular domain and one or more protein domains heterologous to ActRIIa. In some embodiments, the ActRIIa protein is a fusion protein comprising an Fc domain of an immunoglobulin. In some embodiments, the Fc domain of the immunoglobulin is an Fc domain of an IgG1 immunoglobulin. In some embodiments, the ActRIIa fusion protein further comprises a linker domain positioned between the ActRIIa protein domain and the one or more heterologous domains (e.g., an Fc immunoglobulin domain). In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ActRIIa fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 32. In some embodiments, the ActRIIa fusion protein comprises the amino acid sequence of SEQ ID NO: 32. In some embodiments, the ActRIIa fusion protein consists of the amino acid sequence of SEQ ID NO: 32. In some embodiments, the ActRIIa fusion protein consists of the amino acid sequence of SEQ ID NO: 41. In some embodiments, the ActRIIa fusion protein consists of the amino acid sequence of SEQ ID NO: 32 or 41. In some embodiments, the ActRIIa fusion protein consists of a variant of the amino acid sequence set forth in SEQ ID NO: 32, wherein the sequence is lacking the C-terminal lysine residue of SEQ ID NO: 32. In some embodiments, the ActRIIa fusion protein variant lacking the C-terminal lysine residue comprises or consists of the amino acid sequence of SEQ ID NO:41. In some embodiments, the ActRIIa fusion protein is part of a homodimer protein complex. In some embodiments, the ActRIIa fusion protein is glycosylated. In some embodiments, the ActRIIa fusion protein has a glycosylation pattern obtainable by expression in a Chinese hamster ovary cell.

The pharmaceutical formulations described herein comprise an ActRIIa fusion protein and one or more pharmaceutical additives and/or excipients. In certain embodiments, the pharmaceutical formulations described herein comprise a human ActRIIa fusion protein or a variant thereof, a buffer, a surfactant and a stabilizer.

In some embodiments, one or more of the pharmaceutical additives and/or excipients is a buffering agent. In some embodiments, the buffering agent is selected to be physiologically compatible and to maintain a pH above 2. In some embodiments, the buffering agent is selected to be physiologically compatible and capable of maintaining a reconstituted lyophilized solution described herein at a pH above 2. In some embodiments, the buffering agent is selected to be physiologically compatible and to maintain a pH of the pharmaceutical formulation at a pH of 4.0, 4.1, 4.2, 4.3, 4.4, 4.5, 4.6, 4.7, 4.8, 4.9, 5.0, 5.1, 5.2, 5.3, 5.4, 5.5, 5.6 5.7, 5.8, 5.9 6.0, 6.1, 6.2, 6.3, 6.4, 6.5, 6.6 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In some embodiments, the buffering agent is selected to be physiologically compatible and to maintain a pH of the pharmaceutical formulation between 5-7 (i.e., 5.0-7.0). In some embodiments, the buffering agent is selected to be physiologically compatible and to maintain a pH of the pharmaceutical formulation at a pH of 5.5, 5.6 5.7, 5.8, 5.9 6.0, 6.1, 6.2, 6.3, 6.4, 6.5, 6.6 6.7, 6.8, 6.9 or 7.0. In some embodiments, the buffering agent is selected to be physiologically compatible and to maintain a pH of the pharmaceutical formulation between at a pH of 5.5-6.5 (i.e., from 5.5 to 6.5). In some embodiments, the buffering agent is selected to be physiologically compatible and to maintain a pH of the pharmaceutical formulation of 5.8.

In some embodiments, the buffering agent comprises organic acids, succinate, phosphate, acetate, citrate, citric acid, Tris, HEPES, amino acids, or mixtures of amino acids. In certain embodiments, the buffer is selected from the group consisting of sodium citrate, succinate, and histidine. In some embodiments, the buffering agent comprises citrate. In certain embodiments, wherein the buffer is citrate, the citrate buffer comprises tri-sodium citrate dihydrate and citric acid monohydrate. In some embodiments, the buffering agent comprises tri-sodium citrate dihydrate. In some embodiments, the buffering agent comprises citric acid monohydrate. In some embodiments, the buffering agent comprises tri-sodium citrate dihydrate and citric acid monohydrate. In certain embodiments, wherein the protein has a negative charge due to the presence of glycans, the buffer is not histidine.

In some embodiments, the buffering agent is present in an amount of between 4 mM and 50 mM. In some embodiments, the buffering agent is present in a concentration of at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In some embodiments, the pharmaceutical formulation comprises at least 10 mM of buffering agent.

In some embodiments, the buffering agent is a citrate buffer that maintains a pH of the pharmaceutical formulation at a pH from about 5.5 to about pH 6.5. In some embodiments, the buffering agent is a succinate buffer that maintains a pH of the pharmaceutical formulation at a pH from about 5.5 to about pH 6.5. In some embodiments, the buffering agent is a histidine buffer that maintains a pH of the pharmaceutical formulation at a pH from about 5.5 to about pH 6.0. In some embodiments, the buffering agent is a citrate buffer that maintains a pH of the pharmaceutical formulation at a pH of about 5.8. In some embodiments, the buffering agent is a succinate buffer that maintains a pH of the pharmaceutical formulation at a pH of about 5.8.

In some embodiments, the buffering agent is 10 mM of a citrate buffer that maintains a pH of the pharmaceutical formulation at a pH from about 5.5 to about pH 6.5. In some embodiments, the buffering agent is 10 mM of a succinate buffer that maintains a pH of the pharmaceutical formulation at a pH from about 5.5 to about pH 6.5. In some embodiments, the buffering agent is 10 mM of a histidine buffer that maintains a pH of the pharmaceutical formulation at a pH from about 5.5 to about pH 6.0. In some embodiments, the buffering agent is 10 mM of a citrate buffer that maintains a pH of the pharmaceutical formulation at a pH of about 5.8. In some embodiments, the buffering agent is 10 mM of a succinate buffer that maintains a pH of the pharmaceutical formulation at a pH of about 5.8.

In some embodiments, one or more of the pharmaceutical additives and/or excipients is a surfactant. In some embodiments, the surfactant is selected from the group consisting of: sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt, benzalkonium chloride, benzethonium chloride, cetylpyridinium chloride monohydrate, hexadecyltrimethylammonium bromide, CHAPS, CHAPSO, SB3-10, SB3-12, digitonin, Triton X-100, Triton X-114, TWEEN-20, TWEEN-80, lauromacrogol 400, polyoxyl 40 stearate, polyoxyethylene hydrogenated castor oil 10, 40, 50 and 60, glycerol monostearate, polysorbate 40, polysorbate 60, polysorbate 65, polysorbate 80, and soy lecithin. In some embodiments, the surfactant is polysorbate 80 or polysorbate 20. In some embodiments, the surfactant is polysorbate 80.

In some embodiments, the surfactant is present in an amount of 0.05-0.3 mg/mL. In some embodiments, the pharmaceutical formulation comprises at least 0.001, 0.002, 0.003, 0.004, 0.005, 0.01, 0.02, 0.03, 0.05, 0.06, 0.07, 0.08, 0.09, 0.1, 0.2, 0.3, 0.4, 0.5, 0.6, 0.7, 0.8 0.9, or 1.0% weight/volume of surfactant. In some embodiments, the pharmaceutical formulation comprises 0.01 to 0.05% weight/volume surfactant. In some embodiments, the pharmaceutical formulation comprises at least 0.02% weight/volume surfactant. In some embodiments, the pharmaceutical formulation comprises 0.02% weight/volume surfactant. In some embodiments, the pharmaceutical formulation comprises 0.02% polysorbate 80 or polysorbate 20.

In some embodiments, one or more of the pharmaceutical additives and/or excipients is a stabilizer. In some embodiments, the stabilizer is selected from the group consisting of: sucrose, trehalose, mannose, maltose, lactose, glucose, raffinose, cellobiose, gentiobiose, isomaltose, arabinose, glucosamine, fructose, mannitol, sorbitol, poly-hydroxy compounds, polysaccharides, dextran, starch, hydroxyethyl starch, cyclodextrins, N-methyl pyrollidene, cellulose, and hyaluronic acid. In some embodiments, the stabilizer is sucrose.

In some embodiments, the stabilizer is present in the pharmaceutical formulation at a concentration between 2-16% weight/volume. In some embodiments, the pharmaceutical formulation comprises between about 8% to about 10% weight/volume stabilizer. In some embodiments, the pharmaceutical formulation comprises at least 0.005% weight/volume, 0.01% weight/volume, 0.02% weight/volume, 0.03% weight/volume, 0.05% weight/volume, 0.06% weight/volume, 0.07% weight/volume, 0.08% weight/volume, 0.09% weight/volume, 0.1% weight/volume, 0.5% weight/volume, 0.7% weight/volume, 0.8% weight/volume, 0.9% weight/volume, 1.0% weight/volume, 1.2% weight/volume, 1.5% weight/volume, 1.7% weight/volume, 2% weight/volume, 3% weight/volume, 4% weight/volume, 5% weight/volume, 6% weight/volume, 7% weight/volume, 8% weight/volume, 9% weight/volume, 10% weight/volume, 11% weight/volume, 12% weight/volume, 13% weight/volume, 14% weight/volume, 15% weight/volume, 16% weight/volume, 17% weight/volume, 18% weight/volume, 19% weight/volume, or 20% weight/volume stabilizer. In some embodiments, the stabilizer is present at a concentration of at least 8% weight/volume.

In certain embodiments, the pharmaceutical formulations described herein do not contain a salt.

Also described herein are lyophilized pharmaceutical formulations made by lyophilizing the liquid formulations described herein. Also described herein are pharmaceutical formulations, wherein the formulation is reconstituted from a lyophilized formulation.

In some embodiments, the lyophilized pharmaceutical formulation is reconstituted with Sterile Water for Injection. In some embodiments, the lyophilized pharmaceutical formulation is reconstituted with Sterile Water for Injection to a final protein concentration of 10-100 mg/mL. In some embodiments, the lyophilized pharmaceutical formulation is reconstituted with Sterile Water for Injection to a final protein concentration of 45-55 mg/mL. In some embodiments, the lyophilized pharmaceutical formulation is reconstituted with Sterile Water for Injection to a final protein concentration of approximately 10 mg/mL, 15 mg/mL, 20 mg/mL, 25 mg/mL, 30 mg/mL, 35 mg/mL, 45 mg/mL, 46 mg/mL, 47 mg/mL, 48 mg/mL, 49 mg/mL, 50) mg/mL, 51 mg/mL, 52 mg/mL, 53 mg/mL, 54 mg/mL, 55 mg/mL, 60) mg/mL, 65 mg/mL, 70) mg/mL, 75 mg/mL mg/mL, 80 mg/mL, 85 mg/mL, 90) mg/mL, 95 mg/mL or 100 mg/mL. In certain embodiments, the human ActRIIa fusion protein is present in an amount of about 75 mg/mL or less. In certain embodiments, the human ActRIIa fusion protein is present in an amount of about 50 mg/mL.

In some embodiments, the protein is provided in a lyophilized pharmaceutical formulation in a vial. In some embodiments, the lyophilized formulation comprises an ActRIIa fusion protein as defined herein in an amount of 45 mg/vial or 60 mg/vial.

In some embodiments, the lyophilized pharmaceutical formulation is reconstituted with Sterile Water for Injection. In some embodiments, the lyophilized pharmaceutical formulation is reconstituted with Sterile Water for Injection to a final protein concentration of approximately 45 mg/mL, 46 mg/mL, 47 mg/mL, 48 mg/mL, 49 mg/mL, 50) mg/mL, 51 mg/mL, 52 mg/mL, 53 mg/mL, 54 mg/mL, or 55 mg/mL. In some embodiments, the lyophilized pharmaceutical formulation is reconstituted with Sterile Water for Injection to a final protein concentration of approximately 50 mg/mL.

In certain embodiments, the lyophilized formulation is reconstituted with approximately 0.5 mL to 1.8 mL of Sterile Water for Injection. In some embodiments, the lyophilized pharmaceutical formulation is reconstituted with approximately 0.5 mL, 0.56 mL, 0.58 mL, 0.6 mL, 0.62 mL, 0.64 mL, 0.66 mL, 0.68 mL, 0.70 mL, 0.72 mL, 0.74 mL, 0.76 mL, 0.78 mL, 0.8 mL, 0.8 2 mL, 0.84 mL, 0.86 mL, 0.88 mL, 0.9 mL, 0.9 2 mL, 0.94 mL, 0.96 mL, 0.98 mL, 1 mL, 1.1 mL, 1.15 mL, 1.2 mL, 1.25 mL, 1.3 mL, 1.35 mL, 1.4 mL, 1.45 mL, 1.5 mL, 1.55 mL, 1.6 mL, 1.65 mL, 1.7 mL, 1.75 mL, or 1.8 mL of Sterile Water for Injection. In some embodiments, the lyophilized pharmaceutical formulation comprises an ActRIIa fusion protein that is provided in an amount of 45 mg/vial, and the formulation is reconstituted with 0.9 mL, 0.95 mL, 1 mL or 1.1 mL of Sterile Water for Injection. In some embodiments, the lyophilized pharmaceutical formulation comprises an ActRIIa fusion protein that is provided in an amount of 45 mg/vial, and the formulation is reconstituted with 1.1 mL of Sterile Water for Injection. In some embodiments, the lyophilized pharmaceutical formulation comprises an ActRIIa fusion protein that is provided in an amount of 60 mg/vial, and wherein the formulation is reconstituted with 0.90 mL, 0.95 mL, 1.00 mL, 1.05 mL, 1.10 mL, 1.15 mL, 1.20 mL, 1.25 mL, 1.30 mL, 1.35 mL, 1.40 mL, 1.45 mL or 1.50 mL of Sterile Water for Injection. In some embodiments, the lyophilized pharmaceutical formulation comprises an ActRIIa fusion protein that is provided in an amount of 60 mg/vial, and wherein the formulation is reconstituted with 1.30 mL of Sterile Water for Injection.

In certain embodiments, the lyophilized formulation is provided in an amount of 45 mg/vial, and wherein the formulation is reconstituted with between 0.65 mL to 0.75 mL of Sterile Water for Injection. In certain embodiments, the lyophilized formulation is provided in an amount of 60 mg/vial, and wherein the formulation is reconstituted with between 0.65 mL to 1.75 mL of Sterile Water for Injection.

In certain embodiments, the lyophilized pharmaceutical formulation comprises a human ActRIIa fusion protein of SEQ ID NO: 32 or a variant of SEQ ID NO:32 lacking the C-terminal lysine (SEQ ID NO: 41), a buffer, a surfactant and a stabilizer, wherein the buffering agent is selected to be physiologically compatible and to maintain a pH of 5.8 when reconstituted with Sterile Water for Injection. In certain embodiments, the buffering agent comprises citrate. In certain, the stabilizer is sucrose. In certain embodiments, the surfactant is polysorbate 20 or polysorbate 80. In certain embodiments, the surfactant is polysorbate 80. In certain embodiments, the lyophilized pharmaceutical formulation comprises sotatercept, a buffer, a surfactant and a stabilizer, wherein the buffering agent is selected to be physiologically compatible and to maintain a pH of 5.8 when reconstituted with Sterile Water for Injection

In certain embodiments, the lyophilized pharmaceutical formulation comprises a human ActRIIa fusion protein comprising the amino acid sequence of SEQ ID NO: 32 or comprising the amino acid sequence of SEQ ID NO: 41, citrate, polysorbate 80, and sucrose. In some embodiments, the human ActRIIa fusion protein consists of the amino acid sequence of SEQ ID NO: 41. In some embodiments, the lyophilized formulation comprises a human ActRIIa fusion protein comprising the amino acid sequence of SEQ ID NO: 32 and a human ActRIIa fusion protein comprising the amino acid sequence of SEQ ID NO:41. In some embodiments, the lyophilized pharmaceutical formulation comprises sotatercept.

In certain embodiments, the lyophilized pharmaceutical formulation comprises 55.0 mg of a human ActRIIa fusion protein comprising the amino acid sequence of of SEQ ID NO: 32 or a variant of SEQ ID NO:32 lacking the C-terminal lysine residue (e.g., comprising the amino acid sequence of SEQ ID NO: 41), 0.48 mg of citric acid monohydrate, 2.56 mg tri-sodium citrate dihydrate, 0.22 mg polysorbate 80, and 88.0 mg sucrose. In some embodiments, the human ActRIIa fusion protein consists of the amino acid sequence of SEQ ID NO:41. In some embodiments, the lyophilized formulation comprises a human ActRIIa fusion protein comprising the amino acid sequence of SEQ ID NO: 32 and a human ActRIIa fusion protein comprising the amino acid sequence of SEQ ID NO:41. In some embodiments, the lyophilized pharmaceutical formulation comprises sotatercept.

In certain embodiments, the lyophilized pharmaceutical formulation comprises a human ActRIIa fusion protein of 72.5 mg of SEQ ID NO: 32 or a variant of SEQ ID NO:32 lacking the C-terminal lysine residue (SEQ ID NO: 41), 0.64 mg of citric acid monohydrate, 3.37 mg tri-sodium citrate dihydrate, 0.29 mg polysorbate 80, and 116.0 mg sucrose. In some embodiments, the human ActRIIa fusion protein consists of the amino acid sequence of SEQ ID NO:41. In some embodiments, the lyophilized formulation comprises a human ActRIIa fusion protein comprising the amino acid sequence of SEQ ID NO: 32 and a human ActRIIa fusion protein comprising the amino acid sequence of SEQ ID NO:41. In some embodiments, the lyophilized pharmaceutical formulation comprises sotatercept.

In certain embodiments, the reconstituted pharmaceutical formulation comprises 50 mg/mL of a human ActRIIa protein comprising the amino acid sequence of SEQ ID NO: 32 or a human ActRIIa protein comprising the amino acid sequence of SEQ ID NO:41, 10 mM citrate. 0.2 mg/ml polysorbate 80, and 80 mg/ml sucrose at pH 5.8. In some embodiments, the human ActRIIa fusion protein consists of the amino acid sequence of SEQ ID NO:41. In some embodiments, the reconstituted formulation comprises a human ActRIIa fusion protein comprising the amino acid sequence of SEQ ID NO: 32 and a human ActRIIa fusion protein comprising the amino acid sequence of SEQ ID NO:41. In some embodiments, the reconstituted pharmaceutical formulation comprises sotatercept.

In certain embodiments, the pharmaceutical formulations described herein contain a mixture of SEQ ID NO: 32 and a variant of SEQ ID NO:32 lacking the C-terminal lysine residue (SEQ ID NO: 41). In certain embodiments, the pharmaceutical formulations described herein contain a mixture of SEQ ID NO: 32 and a variant of SEQ ID NO:32 lacking the C-terminal lysine residue (SEQ ID NO: 41) wherein the mixture contains 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:32.

In certain embodiments, the pharmaceutical formulations described herein contain a mixture of SEQ ID NO: 32 and a variant of SEQ ID NO:32 lacking the C-terminal lysine residue (SEQ ID NO: 41) wherein the mixture contains about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:32.

In certain embodiments, the pharmaceutical formulations described herein contain a mixture of SEQ ID NO: 32 and a variant of SEQ ID NO:32 lacking the C-terminal lysine residue (SEQ ID NO: 41) wherein the mixture contains 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:41.

In certain embodiments, the pharmaceutical formulations described herein contain a mixture of SEQ ID NO: 32 and a variant of SEQ ID NO:32 lacking the C-terminal lysine residue (SEQ ID NO: 41) wherein the mixture contains about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:41. In certain embodiments, the pharmaceutical formulations described herein contain 100% of SEQ ID NO: 32 by weight. In certain embodiments, the pharmaceutical formulations described herein contain 100% of SEQ ID NO: 41 by weight.

In some embodiments, the pharmaceutical formulation is administered parenterally (e.g., by intravenous infusion). In some embodiments, the pharmaceutical formulation is administered via subcutaneous injection. In certain embodiments, the formulation is contained in a glass vial or injection device.

Also described herein are methods of treating pulmonary arterial hypertension (PAH), comprising administering to a patient in need thereof a pharmaceutical formulation described herein.

Also described herein are uses of a pharmaceutical formulation for the treatment of pulmonary arterial hypertension (PAH), comprising administering to a patient in need thereof the pharmaceutical formulation described herein.

In some embodiments, the disclosure provides a lyophilized pharmaceutical formulation for the treatment of pulmonary arterial hypertension in a subject in need thereof comprising a human ActRIIa fusion protein of SEQ ID NO: 32, citrate, polysorbate 80, and sucrose, wherein the formulation is lyophilized. In some embodiments, the vial comprises a lyophilized pharmaceutical formulation comprising a human ActRIIa fusion protein of SEQ ID NO: 32, citric acid monohydrate, tri-sodium citrate dihydrate, polysorbate 80, and sucrose, wherein the formulation is lyophilized. In some embodiments, the vial comprises a lyophilized pharmaceutical formulation comprising 55 mg of the protein (i.e., ActRIIa fusion protein), 0.48 mg of citric acid monohydrate, 2.56 mg of tri-sodium citrate dihydrate, 0.22 mg polysorbate 80, and 88.0 mg sucrose, wherein the formulation is lyophilized. In some embodiments, the vial comprises a lyophilized pharmaceutical formulation comprising 72.5 mg ActRIIa fusion protein, 0.64 mg citric acid monohydrate, 3.37 mg tri-sodium citrate dihydrate, 0.29 mg polysorbate 80, and 116.0 mg sucrose, wherein the formulation is lyophilized. In some embodiments, the human ActRIIa fusion protein is a variant of SEQ ID NO: 32 lacking the C-terminal lysine. In some embodiments, the human ActRIIa fusion protein comprises the amino acid sequence of SEQ ID NO: 41. In some embodiments, the human ActRIIa fusion protein is sotatercept.

In some embodiments, once reconstituted with Sterile Water, the vial comprises 50 mg/mL of the ActRIIa fusion protein comprising the amino acid sequence of SEQ ID NO: 32, 10 mM citrate, 0.2 mg/ml polysorbate 80, and 8% by weight of sucrose. In some embodiments, the human ActRIIa fusion protein comprises or consists of the amino acid sequence of SEQ ID NO: 41, 10 mM citrate, 0.2 mg/ml polysorbate 80, and 8% by weight of sucrose. In some embodiments, once reconstituted with Sterile Water, the vial comprises 50 mg/mL of sotatercept, 10 mM citrate, 0.2 mg/ml polysorbate 80, and 8% by weight of sucrose.

In some embodiments, the reconstituted pharmaceutical formulation comprises 50 mg/mL of an ActRIIa fusion protein comprising the amino acid sequence of SEQ ID NO: 32, 10 mM citrate, 0.02 mg/ml polysorbate 80, and 80 mg/ml sucrose at pH 5.8. In some embodiments, the human ActRIIa protein comprises the amino acid sequence of SEQ ID NO: 41, 10 mM citrate, 0.02 mg/ml polysorbate 80, and 80 mg/ml sucrose at pH 5.8. In some embodiments, the reconstituted pharmaceutical formulation comprises 50 mg/mL of sotatercept, 10 mM citrate, 0.02 mg/ml polysorbate 80, and 80 mg/ml sucrose at pH 5.8.

### BRIEF DESCRIPTION OF THE DRAWINGS

FIG. 1 shows components of a kit comprising a lyophilized pharmaceutical formulation comprising an ActRIIa fusion protein and an injection device. A vial (1) holds lyophilized pharmaceutical formulation comprising an ActRIIa fusion protein, reconstituted sterile injectable solution, or sterile injectable solution. A prefilled syringe (2) containing a reconstitution solution is used to reconstitute a lyophilized pharmaceutical formulation comprising an ActRIIa fusion protein from (1) into a sterile injectable solution. A vial adapter (3) couples the vial (1) to the pre-filled syringe (2) via attachment to the vial at one end, and attachment to the pre-filled syringe at an opposite end. A syringe (4) and needle (5) are provided for administration of sterile injectable solution. Swab wipes (6) are provided for sterilization of individual kit components.

FIG. 2 shows a multiple sequence alignment of various vertebrate ActRIIa proteins and human ActRIIa (SEQ ID NOs: 62-68).

FIGS. 3A and 3B show the purification of ActRIIa-hFc expressed in CHO cells. The protein purifies as a single, well-defined peak as visualized by sizing column (top panel) and Coomassie stained SDS-PAGE (bottom panel) (left lane: molecular weight standards; right lane: ActRIIa-hFc).

FIGS. 4A and 4B show the binding of ActRIIa-hFc to activin (top panel) and GDF-11 (bottom bpanel), as measured by Biacore™ assay.

FIG. 5 shows the effect of pH and buffer on % monomer species by HP-SEC after 6M at 25° C.

FIG. 6 shows the effect of pH and buffer on binding activity by Biacore™ after 6M 25° C. stability.

FIG. 7 shows the effect of buffer and pH on % HMW, monomer, and % LMW Species by HP-SEC after 3 months on stability.

FIG. 8 shows the effect of pH and buffer on % minor and % major peak by CE-SDS NR after 1 month stability.

FIG. 9 shows the effect of pH and buffer on % acidic, % basic species, and % total main species by Desialylated icIEF after 3 months stability.

FIG. 10 shows the effect of concentration on % monomer species by HP-SEC.

FIG. 11 shows the effect of buffer and concentration on % monomer species by HP-SEC on stability.

FIG. 12 shows the effect of protein and sucrose concentration on purity by HP-SEC on stability.

### DETAILED DESCRIPTION

### Definitions

The terms used in this specification generally have their ordinary meanings in the art, within the context of this disclosure and in the specific context where each term is used. Certain terms are discussed below or elsewhere in the specification, to provide additional guidance to the practitioner in describing the formulations and methods of the disclosure and how to make and use them. The scope or meaning of any use of a term will be apparent from the specific context in which the term is used.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B.” “A” (alone), and “B” (alone). Likewise, the term “and/or” as used in a phrase such as “A, B, and/or C” is intended to encompass each of the following aspects; A, B, and C; A, B, or C; A or C; A or B; B or C; A and C; A and B; B and C; A (alone); B (alone); and C (alone).

Numeric ranges disclosed herein are inclusive of the numbers defining the ranges. For example, pH between 5.0 and 7.0 includes pH 5.0 and pH 7.0 and values between 5.0 and 7.0.

A protein disclosed herein can comprise an amino acid sequence which is not naturally occurring. Such variants necessarily have less than 100% sequence identity or similarity with the starting molecule. In certain embodiments, the variant will have an amino acid sequence from about 75% to less than 100% amino acid sequence identity or similarity with the amino acid sequence of the starting (e.g., naturally-occurring or wild-type) protein, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

### Pharmaceutical Formulations

Provided herein are pharmaceutical formulations comprising a recombinant fusion protein comprising an extracellular domain (ECD) of human activin receptor type-IIA (ActRIIa) proteins or derivatives thereof linked to a constant domain of an immunoglobulin, such as human IgG1 Fc domain, wherein the pharmaceutical formulations are lyophilized. In certain aspects, the disclosure relates to a pharmaceutical formulation comprising an extracellular domain (ECD) of a human ActRIIa protein or a derivative thereof linked to a constant domain of an immunoglobulin, such as human IgG1 Fc domain, wherein the pharmaceutical formulation is reconstituted from a lyophilized formulation into a sterile solution for injection.

In certain aspects, the disclosure relates to a lyophilized pharmaceutical formulation comprising a an extracellular domain (ECD) of human ActRIIa protein or a derivative thereof linked to a constant domain of an immunoglobulin, such as a human IgG1 Fc domain, for reconstitution into a sterile solution for injection. During lyophilization, a liquid formulation is converted from being in an aqueous phase to being in an amorphous solid phase, which is thought to protect the protein from chemical and/or conformational instability. Lyophilization is carried out using techniques common in the art and the lyophilized formulations are optimized for stability, shelf-life, and decreased levels of high molecular weight (HMW) species and aggregates. Tang et al., Pharm Res. 21:191-200. (2004) and Chang et al., Pharm Res. 13:243-9 (1996). The pharmaceutical formulations provided aid in stabilizing the protein against the stresses of manufacturing, shipping and storage. The excipients and additives used in the lyophilized formulations are integral components of a formulation, and therefore need to be safe and well tolerated by patients. For protein drugs, the choice of excipients and additives is particularly important because they can affect both efficacy and immunogenicity of the drug. Excipients and additives are also useful in reducing viscosity of high concentration protein formulations in order to enable their delivery and enhance patient convenience. The formulation excipients and additives disclosed herein provide stability against these stresses. Common excipients are known in the art and can be found in Powell et al., Compendium of Excipients fir Parenteral Formulations (1998), PDA J. Pharm. Sci. Technology, 52:238-311.

In another aspect, provided herein are pharmaceutical formulations comprising an ActRIIa fusion protein. In some embodiments, the formulation is lyophilized. In further embodiments, the formulation is reconstituted from a lyophilized formulation. In certain embodiments, the disclosure provides a pharmaceutical formulation comprising an ActRIIa protein wherein the pharmaceutical formulation is in a lyophilized form in a vial. In certain embodiments, the pharmaceutical formulation comprises between 15 mg to 100 mg of ActRIIa fusion protein. In certain embodiments, the pharmaceutical formulation comprises between 20 mg to 80 mg of ActRIIa fusion protein. In certain embodiments, the pharmaceutical formulation comprises between 40 mg to 70 mg of ActRIIa fusion protein. In certain embodiments, the pharmaceutical formulation comprises about 15 mg, about 17.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, about 50 mg, about 52.5 mg, about 55 mg, about 57.5 mg, about 60 mg, about 62.5 mg, about 65 mg, about 67.5 mg, about 70 mg, about 72.5 mg, about 75 mg, about 77.5 mg, about 80 mg, about 82.5 mg, about 85 mg, about 90 mg, about 92.5 mg, about 95 mg, about 97.5 mg or about 100 mg of the ActRIIa fusion protein.

In certain embodiments, the lyophilized pharmaceutical formulation comprising an ActRIIa fusion protein is provided as a lyophilized powder or cake in a vial. In specific embodiments, the lyophilized pharmaceutical formulation comprises an ActRIIa fusion protein in an amount of 45 mg/vial or 60 mg/vial. In more specific embodiments, each of said 45 mg/vial or said 60 mg/vial is reconstituted with Sterile Water for Injection, each containing a final concentration of 45 to 55 mg/mL of the reconstituted ActRIIa fusion protein. In a more specific embodiment, each of said 45 mg/vial or said 60 mg/vial is reconstituted with Sterile Water for Injection, each containing a final concentration of 50 mg/mL of the reconstituted ActRIIa fusion protein (active pharmaceutical ingredient).

In certain embodiments, the lyophilized pharmaceutical formulation comprises an ActRIIa fusion protein in an amount of 45 mg/vial or 60 mg/vial, and is reconstituted with Sterile Water for Injection. In a more specific embodiment, the lyophilized pharmaceutical formulation provided in each of said 45 mg/vial or said 60 mg/vial is reconstituted with Sterile Water for Injection to a final concentration of approximately 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, or 55 mg/mL of the reconstituted ActRIIa fusion protein. In a more specific embodiment, the lyophilized pharmaceutical formulation provided in each of said 45 mg/vial or said 60 mg/vial is reconstituted with Sterile Water for Injection to a final concentration of 45 mg/mL, 46 mg/mL, 47 mg/mL, 48 mg/mL, 49 mg/mL, 50) mg/mL, 51 mg/mL, 52 mg/mL, 53 mg/mL, 54 mg/mL, or 55 mg/mL of the reconstituted ActRIIa fusion protein. In some embodiments, the lyophilized pharmaceutical formulation provided in each of said 45 mg/vial or said 60 mg/vial is reconstituted with Sterile Water for Injection to a final concentration of approximately 50 mg/ml of the reconstituted ActRIIa fusion protein. In some embodiments, the lyophilized pharmaceutical formulation provided in each of said 45 mg/vial or said 60 mg/vial is reconstituted with Sterile Water for Injection to a final concentration of 50 mg/ml of the reconstituted ActRIIa fusion protein.

In some embodiments, the lyophilized pharmaceutical formulation provided in each of said 45 mg/vial or said 60 mg/vial is reconstituted with 0.5 to 2 mL of Sterile Water for Injection. In some embodiments, the lyophilized pharmaceutical formulation in each of said 45 mg/vial or said 60 mg/vial is reconstituted with approximately 0.5 mL, 0.56 mL, 0.58 mL, 0.6 mL, 0.62 mL, 0.64 mL, 0.66 mL, 0.68 mL, 0.70 mL, 0.72 mL, 0.74 mL, 0.76 mL, 0.78 mL, 0.8 mL, 0.8 2 mL, 0.84 mL, 0.86 mL, 0.88 mL, 0.9 mL, 0.9 2 mL, 0.94 mL, 0.96 mL, 0.98 mL, 1 mL, 1.1 mL, 1.15 mL, 1.2 mL, 1.25 mL, 1.3 mL, 1.35 mL, 1.4 mL, 1.45 mL, 1.5 mL, 1.55 mL, 1.6 mL, 1.65 mL, 1.7 mL, 1.75 mL, or 1.8 mL of Sterile Water for Injection. In some embodiments, the lyophilized pharmaceutical formulation provided in each of said 45 mg/vial or said 60 mg/vial is reconstituted with 0.5 mL, 0.55 mL, 0.56 mL, 0.57 mL, 0.58 mL, 0.59 mL, 0.6 mL, 0.65 mL, 0.66 mL, 0.67 mL, 0.68 mL, 0.69 mL, 0.70 mL, 0.71 mL, 0.72 mL, 0.73 mL, 0.74 mL, 0.75 mL, 0.76 mL, 0.77 mL, 0.78 mL, 0.79 mL, 0.8 mL, 0.85 mL, 0.9 mL, 0.95 mL, 1 mL, 1.1 mL, 1.15 mL, 1.2 mL, 1.25 mL, 1.3 mL, 1.35 mL, 1.4 mL, 1.45 mL, 1.5 mL, 1.51 mL, 1.52 mL, 1.53 mL, 1.54 mL, 1.55 mL, 1.56 mL, 1.57 mL, 1.58 mL, 1.59 mL, 1.6 mL, 1.61 mL, 1.62 mL, 1.63 mL, 1.64 mL, 1.65 mL, 1.66 mL, 1.67 mL, 1.68 mL, 1.69 mL, 1.7 mL, 1.71 mL, 1.72 mL, 1.73 mL, 1.74 mL, 1.75 mL, 1.76 mL, 1.77 mL, 1.78 mL, 1.79 mL, or 1.8 mL of Sterile Water for Injection. In some embodiments, the lyophilized pharmaceutical formulation provided in said 45 mg/vial is reconstituted with 0.8 mL, 0.85 mL, 0.9 mL, 0.95 mL, 1 mL, 1.1 mL, 1.15 mL, 1.2 mL, 1.25 mL, 1.3 mL, 1.35 mL or 1.4 mL 1.75 mL of Sterile Water for Injection. In some embodiments, the lyophilized pharmaceutical formulation provided in said 45 mg/vial is reconstituted with 0.9 mL, 0.95 mL, 1 mL or 1.1 mL of Sterile Water for Injection. In some embodiments, the lyophilized pharmaceutical formulation provided in said 45 mg/vial is reconstituted with 1.1 mL of Sterile Water for Injection. In some embodiments, the lyophilized pharmaceutical formulation provided in said 60 mg/vial is reconstituted with 0.8 mL, 0.85 mL, 0.9 mL, 0.95 mL, 1 mL, 1.1 mL, 1.15 mL, 1.2 mL, 1.25 mL, 1.3 mL, 1.35 mL or 1.4 mL 0.75 mL of Sterile Water for Injection. In some embodiments, the lyophilized pharmaceutical formulation provided in said 60 mg/vial is reconstituted with 1 mL, 1.1 mL, 1.2 mL, or 1.3 mL of Sterile Water for Injection. In some embodiments, the lyophilized pharmaceutical formulation provided in said 60 mg/vial is reconstituted with 1.3 mL of Sterile Water for Injection.

In some embodiments, the lyophilized pharmaceutical formulations provided herein comprises an ActRIIa fusion protein and one or more pharmaceutical additives and/or excipients. In certain embodiments, the one or more pharmaceutical additives and/or excipients comprises a buffer, stabilizer, and a surfactant. Buffering agents may be selected to maintain the pH of the formulation during processing and upon reconstitution. Stabilizers may include cryo and lyoprotectants, such as polyols, sugars, and polysaccharides, and may be selected to protect the formulation from freeze/thaw cycle stresses and stabilize the formulation in the freeze-dried state. Surfactants may be selected based on their ability to serve as an emulsifier, wetter, solubilizer and/or dispersant.

The formulations provided herein comprise buffering agents, surfactants, and sugars, which are described in greater detail below.

A person having ordinary skill in the art would recognize that the concentrations of the excipients described herein share an interdependency within a particular formulation. By way of example, the concentration of a bulking agent is, in one aspect, lowered where, e.g., there is a high protein concentration. Excipients and other additives are added to impart or enhance manufacturability and/or final product quality, such as the stability and delivery of a drug product (e.g., protein). The formulations provided herein comprise suitable excipients that enhance stability, and safety.

### Buffering Agents

Typically, the stability of a pharmacologically active protein formulation is observed to be maximal in a narrow pH range. This pH range of optimal stability needs to be identified early during pre-formulation studies. Several approaches, such as accelerated stability studies and calorimetric screening studies, are useful in this endeavor (Remmele R. L. Jr., et al., Biochemistry, 38(16): 5241-7 (1999)). Once a formulation is finalized, the protein must be manufactured and maintained throughout its shelf-life. Hence, buffering agents are almost always employed to control pH in the formulation.

Several factors must be considered when choosing a buffering agent. First and foremost, the buffer species and its concentration must be defined based on its pKa and the desired formulation pH. Equally important is to ensure that the buffer is compatible with the protein and other formulation excipients, and does not catalyze any degradation reactions. A third important aspect to be considered is the sensation of stinging and irritation the buffer may induce upon administration. The potential for stinging and irritation is greater for drugs that are administered via the subcutaneous (SC) or intramuscular (IM) routes, where the drug solution remains at the site for a relatively longer period of time than when administered by the IV route where the formulation gets diluted rapidly into the blood upon administration. For formulations that are administered by direct IV infusion, the total amount of buffer (and any other formulation component) needs to be monitored.

Buffers for lyophilized formulations require additional consideration. For example, particular buffers such as sodium phosphate have a propensity to crystallize out of the protein amorphous phase during freezing resulting in shifts in pH. In certain embodiments, exemplary buffering agents used to buffer the pharmaceutical formulations as set forth herein include, but are not limited to organic acids, succinate, phosphate, acetate, citrate, Tris, HEPES, and amino acids or mixtures of amino acids, including, but not limited to aspartate, arginine and glycine. In some embodiments, the buffering agent comprises tri-sodium citrate dihydrate. In some embodiments, the buffering agent comprises citric acid monohydrate. In some embodiments, the buffering agent comprises citrate. In one embodiment, the buffering agent comprises tri-sodium citrate dihydrate and citric acid monohydrate. In another embodiment, the buffering agents are tri-sodium citrate dihydrate and citric acid monohydrate. In certain embodiments, wherein the protein has a negative charge due to the presence of glycans, the buffer is not histidine. In some embodiments, the buffer comprises citrate, succinate or histidine.

In certain embodiments, the amount of buffer in the lyophilized formulation is between 0.3 mg and 5 mg. In certain embodiments, the amount of buffer in the lyophilized formulation is 0.3 mg, 0.4 mg, 0.5 mg, 1.0 mg, 1.5 mg, 2.0 mg, 2.5 mg, 3.0 mg, 3.5 mg, 4.0 mg, 4.5 mg, or 5.0) mg. In certain embodiments, the amount of buffer in the lyophilized formulation is about 0.5 mg, about 1.0 mg, about 1.5 mg, about 2.0 mg, about 2.5 mg, about 3.0 mg, about 3.5 mg, about 4.0 mg, about 4.5 mg, or about 5.0 mg.

In certain embodiments, the buffer comprises citric acid monohydrate and tri-sodium citrate dihydrate, wherein the amount of citric acid monohydrate is between 0.1 mg and 1.0 mg. In certain embodiments, the amount of citric acid monohydrate is 0.1 mg, 0.2 mg, 0.3 mg, 0.4, mg 0.5 mg, 0.6 mg, 0.7 mg, 0.8 mg, 0.9 mg or 1.0 mg. In certain embodiments, the amount of citric acid monohydrate is about 0.1 mg, about 0.2 mg, about 0.3 mg, about 0.4, mg, about 0.5 mg, about 0.6 mg, about 0.7 mg, about 0.8 mg, about 0.9 mg or about 1.0 mg. In certain embodiments, the amount of citric acid monohydrate is 0.48 mg. In certain embodiments, the amount of citric acid monohydrate is 0.48 mg. In certain embodiments, the amount of citric acid monohydrate is 0.48 mg. In certain embodiments, the amount of citric acid monohydrate is 0.64 mg.

In certain embodiments, the buffer comprises citric acid monohydrate and tri-sodium citrate dihydrate, wherein the amount of tri-sodium citrate dihydrate is between 1.0 mg and 5.0 mg. In certain embodiments, the amount of tri-sodium citrate dihydrate is 1.0 mg, 2.0 mg, 2.5 mg, 3.0 mg, 4.0, mg or 5.0 mg. In certain embodiments, the amount of tri-sodium citrate dihydrate is about 1.0 mg, about 2.0 mg, about 2.5 mg, about 3.0 mg, about 4.0, mg or about 5.0 mg. In certain embodiments, the amount of tri-sodium citrate dihydrate is 2.56 mg. In certain embodiments, the amount of tri-sodium citrate dihydrate is 3.37 mg.

In one embodiment, the buffering agent present in the formulation is selected to be physiologically compatible and to maintain a desired pH of the pharmaceutical formulation when reconstituted with Sterile Water for Injection. In another embodiment, the pH of the solution is above 2. In another embodiment, the pH of the solution is between pH 2.0 and pH 12.0. For example, in various embodiments, the pH of the reconstituted solution is 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0. In some embodiments, the pH of the solution is between pH 5 and pH 7 when reconstituted in solution. In some embodiments, the pH of the reconstituted solution is 5.5, 5.6, 5.7, 5.8, 5.9 6.0, 6.1, 6.2, 6.3, 6.4, 6.5, 6.6 6.7, 6.8, 6.9 or 7.0. In one embodiment the pH of the reconstituted solution is ≤6.5. In one embodiment, the pH of the reconstituted solution is from about 5.5 to about 6.5. In one embodiment, the pH of the reconstituted solution is from about 5.3 to about 6.3. In one embodiment, the pH of the reconstituted solution is ≤6.5. In one embodiment, the pH of the reconstituted solution is from about 6.0 to about 6.5. In one embodiment, the pH of the reconstituted solution is 5.8.

In some embodiments, the buffer comprises citrate and the pH of the reconstituted solution is from about 5.5 to about 6.5. In some embodiments, the buffer comprises citrate and the pH of the reconstituted solution is from about 5.3 to about 6.3. In some embodiments, the buffer comprises citrate and the pH of the reconstituted solution is about 5.8.

In some embodiments, the buffer comprises succinate and the pH of the reconstituted solution is from about 5.5 to about 6.5. In some embodiments, the buffer comprises succinate and the pH of the reconstituted solution is from about 5.3 to about 6.3. In some embodiments, the buffer comprises succinate and the pH of the reconstituted solution is about 5.8.

The pH buffering compound may be present in any amount suitable to maintain the pH of the formulation at a predetermined level. When appropriately low levels of buffer are used, crystallization and pH shifts may be avoided. In one embodiment, the concentration of the buffering agent is between 0.1 mM and 500 mM (1 M). For example, it is contemplated that the buffering agent is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In certain embodiments, the buffering agent is 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In one embodiment, the concentration of the buffering agent is between 0.1 mM and 20 mM. In certain embodiments, the buffering agent is 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19 or 20 mM. In certain embodiments, the buffering agent is present in an amount of 10 mM.

In some embodiments, the buffering agent is 10 mM of a citrate buffer that maintains a pH of the pharmaceutical formulation at a pH from about 5.5 to about pH 6.5. In some embodiments, the buffering agent is 10 mM of a succinate buffer that maintains a pH of the pharmaceutical formulation at a pH from about 5.5 to about pH 6.5. In some embodiments, the buffering agent is 10 mM of a histidine buffer that maintains a pH of the pharmaceutical formulation at a pH from about 5.5 to about pH 6.0. In some embodiments, the buffering agent is 10 mM of a citrate buffer that maintains a pH of the pharmaceutical formulation at a pH of about 5.8. In some embodiments, the buffering agent is 10 mM of a succinate buffer that maintains a pH of the pharmaceutical formulation at a pH of about 5.8.

In some embodiments, the buffering agent comprises tri-sodium citrate dihydrate. In some embodiments, the buffering agent comprises citric acid monohydrate. In some embodiments, the buffering agent comprises citrate. In some embodiments, the buffering agent comprises tri-sodium citrate dihydrate and citric acid monohydrate. In certain embodiments, the protein has a negative charge due to the presence of glycans and the buffer is not histidine. In some embodiments, the buffering agent is present in the pharmaceutical formulation at a concentration of at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In some embodiments, the pharmaceutical formulation comprises at least 10 mM buffering agent.

### Stabilizers

In certain embodiments the pharmaceutical formulations provided herein comprise stabilizers. These stabilizers can be classified on the basis of the mechanisms by which they stabilize proteins against various chemical and physical stresses. Some stabilizers are used to alleviate the effects of a specific stress or to regulate a particular susceptibility of a specific protein. Other stabilizers have more general effects on the physical and covalent stabilities of proteins. Given the teachings and guidance provided herein, those skilled in the art will know what amount or range of stabilizer can be included in any particular formulation to achieve a formulation of the disclosure that is likely to promote retention and stability of the ActRIIa protein.

In some embodiments, a stabilizer (or a combination of stabilizers) is added to the formulation to prevent or reduce storage-induced aggregation and chemical degradation. A hazy or turbid solution upon reconstitution normally indicates that the protein has precipitated or at least aggregated. Stabilizers are capable of preventing aggregation, or chemical degradation (for example, autolysis, deamidation, oxidation, etc.). Some stabilizers are also capable of acting as anticoagulants upon administration of the formulation to a patient. In certain embodiments, the pharmaceutical formulations provided herein include stabilizers including but not limited to, sucrose, trehalose, mannose, maltose, lactose, glucose, raffinose, cellobiose, gentiobiose, isomaltose, arabinose, glucosamine, fructose, mannitol, sorbitol, poly-hydroxy compounds, including polysaccharides such as dextran, starch, hydroxyethyl starch, cyclodextrins, N-methyl pyrollidene, cellulose and hyaluronic acid (Carpenter et al., Develop. Biol. Standard 74:225, (1991)). In one embodiment of the disclosure, sucrose is used as a stabilizer.

In certain embodiments, the lyophilized formulations described herein contain a stabilizer in an amount between 50 mg to 150 mg. In certain embodiments, the lyophilized formulations described herein contain a stabilizer in an amount of 50 mg, 60 mg, 70 mg, 80 mg, 90 mg, 100 mg, 110 mg, 120 mg, 130 mg, 140 mg or 150 mg. In certain embodiments, the lyophilized formulations described herein contain a stabilizer in an amount of about 50 mg, about 60 mg, about 70 mg, about 80 mg, about 90 mg, about 100 mg, about 110 mg, about 120 mg, about 130 mg, about 140 mg or about 150 mg.

In certain embodiments, the stabilizer is sucrose in the amount of 88.0 mg. In certain embodiments, the stabilizer is sucrose in the amount of 116.0 mg.

In certain embodiments the reconstituted formulation comprises a stabilizer at a concentration of about 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM. Likewise, in certain embodiments of the disclosure, the stabilizer is incorporated in a concentration of about 0.005, 0.01, 0.02, 0.03, 0.05, 0.06, 0.07, 0.08, 0.09, 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, or 20% weight/volume.

In some embodiments, the formulation comprises about 8% to about 10% weight/volume sucrose. In some embodiments, the formulation comprises 8-10% weight/volume sucrose. In some embodiments, the formulation comprises 8%, 9% or 10% weight/volume sucrose. In some embodiments, the formulation comprises 8% weight/volume sucrose.

### Surfactants

In certain embodiments, the pharmaceutical formulations provided herein may additionally include surfactants. Surfactants are commonly used in protein formulations to prevent surface-induced degradation. Surfactants are amphipathic molecules with the capability of out-competing proteins for interfacial positions (and/or promote proper refolding of a structurally altered protein molecule). Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serve to prevent protein molecules from adsorbing at the interface. Thereby, surface-induced degradation is minimized. Surfactants contemplated herein include, without limitation, fatty acid esters of sorbitan polyethoxylates, i.e., polysorbate 20 and polysorbate 80. The two differ only in the length of the aliphatic chain that imparts hydrophobic character to the molecules, C-12 and C-18, respectively. Accordingly, polysorbate-80 is more surface-active and has a lower critical micellar concentration than polysorbate-20.

Detergents can also affect the thermodynamic conformational stability of proteins. Non-ionic surfactants are generally useful in protein stabilization. Ionic surfactants (detergents) normally destabilize proteins. Here again, the effects of a given detergent excipient will be protein specific. For example, polysorbates have been shown to reduce the stability of some proteins and increase the stability of others. Detergent destabilization of proteins can be rationalized in terms of the hydrophobic tails of the detergent molecules that can engage in specific binding with partially or wholly unfolded protein states. These types of interactions could cause a shift in the conformational equilibrium towards the more expanded protein states (i.e., increasing the exposure of hydrophobic portions of the protein molecule in complement to binding polysorbate). Alternatively, if the protein native state exhibits some hydrophobic surfaces, detergent binding to the native state may stabilize that conformation. Another aspect of polysorbates is that they are inherently susceptible to oxidative degradation. Often, as raw materials, they contain sufficient quantities of peroxides to cause oxidation of protein residue side-chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization.

Surfactants are also added in appropriate amounts to prevent surface related aggregation phenomenon during freezing and drying (Chang, B, J. Pharm. Sci. 85:1325, (1996)). Thus, exemplary surfactants include, without limitation, anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. Surfactants also include, but are not limited to lauromacrogol 400, polyoxyl 40 stearate, polyoxyethylene hydrogenated castor oil 10, 40, 50 and 60, glycerol monostearate, polysorbate 40, polysorbate 60, polysorbate 65 and polysorbate 80, soy lecithin and other phospholipids such as dioleyl phosphatidyl choline (DOPC), dimyristoylphosphatidyl glycerol (DMPG), dimyristoylphosphatidyl choline (DMPC), and (dioleyl phosphatidyl glycerol) DOPG; sucrose fatty acid ester, methyl cellulose and carboxymethyl cellulose. Formulations comprising these surfactants, either individually or as a mixture in different ratios, are therefore further provided. In one embodiment of the present disclosure, the surfactant is polysorbate 80 or polysorbate 20. In one embodiment of the present disclosure, the surfactant is polysorbate 80.

In certain embodiments, the surfactant is polysorbate 80 or polysorbate 20, and the amount of polysorbate 80 or polysorbate 20 is between 0.1 mg and 1.0 mg. In certain embodiments, the amount of polysorbate 80 or polysorbate 20 is 0.1 mg, 0.2 mg, 0.3 mg, 0.4, mg 0.5 mg, 0.6 mg, 0.7 mg, 0.8 mg, 0.9 mg or 1.0 mg. In certain embodiments, the amount of polysorbate 80 or polysorbate 20 is about 0.1 mg, about 0.2 mg, about 0.3 mg, about 0.4, mg, about 0.5 mg, about 1.6 mg, about 0.7 mg, about 0.8 mg, about 0.9 mg or about 1.0 mg. In certain embodiments, the amount of polysorbate 80 is 0.22 mg. In certain embodiments, the amount of polysorbate 80 is 0.29 mg. In certain embodiments, the amount of polysorbate 20 is 0.22 mg. In certain embodiments, the amount of polysorbate 20 is 0.29 mg.

In the reconstituted formulations, the surfactant is at a concentration of about 0.01 to about 0.5 g/L. In various embodiments of the pharmaceutical formulations provided herein, the surfactant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 g/L. Likewise, in certain embodiments of the disclosure, the surfactant is incorporated in a concentration of about 0.001, 0.002, 0.003, 0.004, 0.005, 0.01, 0.02, 0.03, 0.05, 0.06, 0.07, 0.08, 0.09, 0.1, 0.2, 0.3, 0.4, 0.5, 0.7, 0.8 0.9, or 1.0% weight/volume. In certain embodiments of the disclosure, the surfactant is incorporated in a concentration of about 0.01 to about 0.05% weight/volume. In certain embodiments, the surfactant is incorporated in a concentration of about 0.02% weight/volume.

In some embodiments, the surfactant is polysorbate 20 or polysorbate 80. In some embodiments, the surfactant is polysorbate 80. In some embodiments, the pharmaceutical formulation comprises 0.05-0.3 mg/mL surfactant. In some embodiments, the pharmaceutical formulation comprises at least 0.001, 0.002, 0.003, 0.004, 0.005, 0.01, 0.02, 0.03, 0.05, 0.06, 0.07, 0.08, 0.09, 0.1, 0.2, 0.3, 0.4, 0.5, 0.6, 0.7, 0.8 0.9, or 1.0% weight/volume surfactant. In some embodiments, the surfactant is present in the pharmaceutical formulation at a concentration of at least 0.02% weight/volume. In certain embodiments, polysorbate 80 or polysorbate 20 is incorporated in a concentration of about 0.01 to about 0.05% weight/volume. In certain embodiments, polysorbate 80) or polysorbate 20 is incorporated in a concentration of about 0.02% weight/volume.

### ActRIIa Formulations

In some embodiments of the pharmaceutical formulations provided herein, the vials of ActRIIa fusion protein provided herein comprise an ActRIIa fusion protein and one or more pharmaceutical additives and/or excipients. In some embodiments, the pharmaceutical formulations are lyophilized. In some embodiments, the formulations are reconstituted from a lyophilized formulation. In one embodiment of the pharmaceutical formulations provided herein, a vial of a lyophilized pharmaceutical formulation comprises 55 mg of ActRIIa fusion protein; 0.48 mg citric acid monohydrate, 2.56 mg tri-sodium citrate dehydrate, 1.22 mg polysorbate 80, and 88 mg sucrose. In another embodiment, said vial is rehydrated with 1.0 mL liquid, e.g., Sterile Water for Injection. In another embodiment of the pharmaceutical formulations provided herein, a vial comprises a 72.5 mg ActRIIa fusion protein; 0.64 mg citric acid monohydrate, 3.37 mg tri-sodium citrate dehydrate, 0.29 mg polysorbate 80, and 116 mg sucrose, wherein the formulation is lyophilized. In another embodiment, said vial is rehydrated with 1.3 mL liquid, e.g., sterile water for injection. In further embodiments, said vials comprise one, two, or all three of citrate, e.g., 10 mM citrate; sucrose, e.g., 8% (weight/volume) sucrose; and/or polysorbate 80, e.g., at pH 5.0 to 7.0, e.g., 0.02% (weight/volume) polysorbate 80 at pH 5.8.

In some embodiments, once reconstituted with Sterile Water, the vial comprises 50 mg/mL of the ActRIIa fusion protein of SEQ ID NO: 32 or SEQ ID NO:41, 10 mM citrate, 0.2 mg/ml polysorbate 80, and 8% by weight of sucrose. In some embodiments, once reconstituted with Sterile Water, the vial comprises 50 mg/mL of sotatercept, 10 mM citrate, 0.2 mg/ml polysorbate 80, and 8% by weight of sucrose.

In some embodiments, the reconstituted pharmaceutical formulation comprises 50 mg/mL of SEQ ID NO: 32 or SEQ ID NO:41, 10 mM citrate, 0.2 mg/ml polysorbate 80, and 80 mg/ml sucrose at pH 5.8. In some embodiments, the reconstituted pharmaceutical formulation comprises 50 mg/mL of sotatercept, 10) mM citrate, 1.2 mg/ml polysorbate 80, and 80 mg/ml sucrose at pH 5.8.

In certain embodiments the dose is administered parenterally. In some embodiments, the dose is administered via subcutaneous injection. In some embodiments, the dose is administered via intradermal injection. In some embodiments, the dose is administered via intramuscular injection. In some embodiments, the dose is administered via intravenous injection. In some embodiments, the dose is self-administered.

### Kits

The present disclosure provides a kit comprising a lyophilized pharmaceutical formulation and an injection device. In certain embodiments, the lyophilized pharmaceutical formulation comprises an ActRIIa protein (e.g., a protein that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 9 or SEQ ID NO: 32), or fragments, functional variants, or modified forms thereof. In certain embodiments, the protein binds to one or more ligands selected from the group consisting of activin A, activin B, and GDF11. In certain such embodiments, the protein further binds to one or more ligands selected from the group consisting of BMP10, GDF8, and BMP6. In certain embodiments, the protein binds to activin and/or GDF11.

In some embodiments, the lyophilized pharmaceutical formulation comprises a protein that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 9 or SEQ ID NO: 32. In certain such embodiments, the protein comprises an amino acid sequence that is least 90%, 95%, or 99% identical to SEQ ID NO: 9 or SEQ ID NO: 32, wherein the protein binds to activin and/or GDF11. In certain embodiments, the protein comprises the amino acid sequence of SEQ ID NO: 9 or SEQ ID NO: 32. In other embodiments, the protein consists of the amino acid sequence of SEQ ID NO: 9 or SEQ ID NO: 32. In certain embodiments, the protein comprises the amino acid sequence of SEQ ID NO: 41. In other embodiments, the protein consists of the amino acid sequence of SEQ ID NO: 41.

In some embodiments, the lyophilized pharmaceutical formulation comprises a protein that comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 32. In certain embodiments, the protein consists essentially of the amino acid sequence of SEQ ID NO: 32. In other embodiments, the protein consists of the amino acid sequence of SEQ ID NO: 32. In other embodiments, the protein consists of a variant of SEQ ID NO: 32 lacking the C-terminal lysine residue (i.e., SEQ ID NO:41).

In certain embodiments of the foregoing, the lyophilized pharmaceutical formulation comprises a fusion protein further comprising an Fc domain of an immunoglobulin. In certain such embodiments, the Fc domain of the immunoglobulin is an Fc domain of an IgG1 immunoglobulin. In other embodiments, the fusion protein further comprises a linker domain positioned between the protein domain and the Fc domain of the immunoglobulin. In certain embodiments, the linker domain is a polyglycine linker.

In certain embodiments, the protein is part of a homodimer protein complex.

In certain embodiments, the protein is glycosylated.

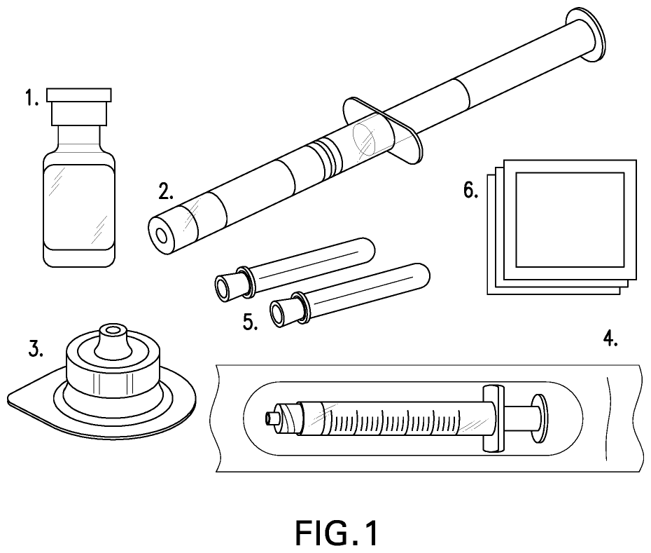

The present disclosure further provides a kit comprising a sterile lyophilized pharmaceutical formulation comprising a protein as disclosed herein and an injection device. In some embodiments of the kits disclosed herein, the sterile lyophilized pharmaceutical formulation comprising a protein is pre-filled in one or more containers, such as one or more vials FIG. 1.

In certain embodiments, the pH range for the sterile lyophilized pharmaceutical formulation comprising an ActRIIa fusion protein is from 5 to 7. In some embodiments, the sterile lyophilized pharmaceutical formulation comprising an ActRIIa fusion protein further comprises a buffering agent. In some embodiments, the buffering agent is added in an amount of at least 10 mM. In some embodiments, the buffering agent is added in an amount in the range of between about 10 mM to about 200 mM. In some embodiments, the buffering agent comprises citrate.

In some embodiments, the pharmaceutical formulation further comprises a surfactant. In some embodiments, the surfactant comprises a polysorbate. In some embodiments, the surfactant comprises polysorbate 20 or polysorbate 80. In some embodiments, the surfactant comprises polysorbate 80.

In some embodiments, the lyophilized pharmaceutical formulation further comprises a lyoprotectant. In some embodiments, the lyoprotectant comprises a sugar, such as a disaccharide (e.g., sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the lyophilized pharmaceutical formulation comprises the lyoprotectant and protein in a weight ratio of at least 1:1 protein to lyoprotectant. In some embodiments, the lyophilized pharmaceutical formulation comprises the lyoprotectant and protein in a weight ratio of from 1:1 to 1:10 protein to lyoprotectant. In some embodiments, the lyophilized pharmaceutical formulation comprises the lyoprotectant and protein in a weight ratio of 1:1, 1:2, 1:3, 1:4, 1:5, 1:6, 1:7, 1:8, 1:9, or 1:10 protein to lyoprotectant. In some embodiments, the lyophilized pharmaceutical formulation comprises the lyoprotectant and protein in a weight ratio of 1:6 protein to lyoprotectant. In certain embodiments of the foregoing, the lyophilized pharmaceutical formulation comprises lyoprotectant in an amount sufficient to stabilize the protein.

In certain embodiments of the kits disclosed herein, the injection device comprises a syringe, as shown in FIG. 1. In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises an aqueous solution such as water, physiologically buffered saline, or other solvent or vehicle such as glycol, glycerol, an oil or an injectable organic ester. In some embodiments, the pharmaceutically acceptable excipient comprises a pharmaceutically acceptable excipient selected from calcium phosphate, calcium carbonate, calcium sulfate, a halite, a metallic oxide, a sugar, a sugar alcohol, starch, glycol, povidone, a mineral hydrocarbon, an acrylic polymer, a fatty alcohol, mineral stearate, glycerin, and/or a lipid. In certain embodiments, the reconstitution solution comprises pharmaceutically acceptable sterile isotonic aqueous or nonaqueous solutions, dispersions, suspensions or emulsions. In certain such embodiments, the reconstitution solution comprises an antioxidant, a buffer, a bacteriostat, and/or a solute which renders the formulation isotonic with the blood of the intended recipient. In other embodiments, the reconstitution solution comprises a suspending or thickening agent.

In certain embodiments of the kits disclosed herein, the kit further comprises a vial adapter, as shown in FIG. 1. In some embodiments, the vial (1) pre-filled with a lyophilized pharmaceutical formulation of the invention attaches to one end of the vial adapter (3). In some embodiments, the syringe pre-filled (2) with a reconstitution solution as disclosed herein attaches to an end of the vial adapter (3). In some embodiments, the syringe pre-filled (2) with a reconstitution solution as disclosed herein and the vial (1) pre-filled with the lyophilized pharmaceutical formulation are attached to opposite ends of the vial adapter (3). In some embodiments, the reconstitution solution is transferred from the pre-filled syringe to the vial. In some embodiments, transfer of the reconstitution solution to the vial pre-filled with the lyophilized pharmaceutical formulation reconstitutes the lyophilized formulation into a sterile injectable solution. In some embodiments, the lyophilized formulation is reconstituted into a sterile injectable solution. In some embodiments, the lyophilized formulation is reconstituted into a sterile injectable solution prior to use.

In other embodiments of the kits disclosed herein, the kit further comprises a pump apparatus. In certain embodiments, the pump apparatus comprises an electromechanical pumping assembly. In certain embodiments, the pump apparatus comprises a reservoir for holding a sterile injectable solution. In certain embodiments, the reservoir holds 1 mL of sterile injectable solution. In certain embodiments, the pump apparatus comprises one or more vials or cartridges comprising a sterile injectable solution. In certain embodiments, the vials or cartridges are prefilled with sterile injectable solution. In certain embodiments, the vials or cartridges comprise sterile injectable solution reconstituted from a lyophilized formulation. In certain embodiments, the reservoir is coupled to the vial or cartridge. In certain embodiments, the vial or cartridge holds 1-20 mL of sterile injectable solution. In certain embodiments, the electromechanical pumping assembly comprises a pump chamber. In certain embodiments, the electromechanical pumping assembly is coupled to the reservoir. In certain embodiments, the sterile injectable solution is received from the reservoir into the pump chamber. In some embodiments, the electromechanical pumping assembly comprises a plunger that is disposed such that sterile injectable solution in the pump chamber is in direct contact with the plunger. In certain embodiments, a sterile injectable solution is received from the reservoir into the pump chamber during a first pumping phase, and is delivered from the pump chamber to a subject during a second pumping phase. In certain embodiments, the electromechanical pumping assembly comprises control circuitry. In certain embodiments, control circuitry drives the plunger to (a) draw the sterile injectable solution into the pump chamber during the first pumping phase and (b) deliver the sterile injectable solution from the pump chamber in a plurality of discrete motions of the plunger during the second pumping phase, thereby delivering the therapeutic substance to the subject in a plurality of controlled and discrete dosages throughout the second pumping phase. In certain embodiments, a cycle of alternating the first and second pumping phases is repeated until a desired dose is administered. In certain embodiments, the pump apparatus is coupled to a wearable patch. In certain embodiments, the pump apparatus is a wearable pump apparatus.

The present disclosure provides a kit used for reconstituting a lyophilized pharmaceutical formulation into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution parenterally. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight-based dose.

### ActRIIa Polypeptides

In certain embodiments, the present disclosure relates to ActRIIa fusion proteins. As used herein, the term “ActRIIa” refers to a family of activin receptor type IIA (ActRIIa) proteins from any species and variants derived from such ActRIIa fusion proteins by mutagenesis or other modification. Reference to ActRIIa herein is understood to be a reference to any one of the currently identified forms. Members of the ActRIIa family are generally transmembrane proteins, composed of a ligand-binding extracellular domain comprising a cysteine-rich region, a transmembrane domain, and a cytoplasmic domain with predicted serine/threonine kinase activity.

The term “ActRIIa fusion protein” includes proteins comprising any naturally occurring protein of an ActRIIa family member as well as any variants thereof (including mutants, fragments, fusions, and peptidomimetic forms) that retain a useful activity. Examples of such variant ActRIIa fusion proteins are provided throughout the present disclosure as well as in International Patent Application Publication Nos. WO 2006/012627 and WO 2007/062188, which are incorporated herein by reference in their entirety. Numbering of amino acids for all ActRIIa-related proteins described herein is based on the numbering of the human ActRIIa precursor protein sequence provided below (SEQ ID NO: 9), unless specifically designated otherwise.

The canonical human ActRIIa precursor protein sequence is as follows:

(SEQ ID NO: 9)

```
    1 MGAAAKLAFA VFLISCSSGA ILGRSETQEC
        LFFNANWEKD RTNQTGVEPC
  51 YGDKDKRRHC FATWKNISGS IEIVKQGCWL
        DDINCYDRTD CVEKKDSPEV
101 YFCCCEGNMC NEKFSYFPEM EVTQPTSNPV
        TPKPPYYNIL LYSLVPLMLI
151 AGIVICAFWV YRHHKMAYPP VLVPTQDPGP
        PPPSPLLGLK PLQLLEVKAR
201 GRFGCVWKAQ LLNEYVAVKI FPIQDKQSWQ
        NEYEVYSLPG MKHENILQFI
251 GAEKRGTSVD VDLWLITAFH EKGSLSDFLK
        ANVVSWNELC HIAETMARGL
301 AYLHEDIPGL KDGHKPAISH RDIKSKNVLL
        KNNLTACIAD FGLALKFEAG
351 KSAGDTHGQV GTRRYMAPEV LEGAINFORD
        AFLRIDMYAM GLVLWELASR
401 CTAADGPVDE YMLPFEEEIG QHPSLEDMQE
        VVVHKKKRPV LRDYWQKHAG
451 MAMLCETIEE CWDHDAEARL SAGCVGERIT
        QMQRLTNIIT TEDIVTVVTM
501 VTNVDFPPKE SSL
```

The signal peptide is indicated by a single underline; the extracellular domain is indicated in bold font; and the potential, endogenous N-linked glycosylation sites are indicated by a double underline.

The processed (mature) extracellular human ActRIIa protein sequence is as follows:

(SEQ ID NO: 10)
ILGRSETQECLFFNANWEKDRTNQTGVEPCYGDKDKRRHCFA
TWKNISGSIEIVKQGCWLDDINCYDRTDCVEKKDSPEVYFCC
CEGNMCNEKFSYFPEMEVTQPTSNPVTPKPP

The C-terminal “tail” of the extracellular domain is indicated by single underline. The sequence with the “tail” deleted (a 415 sequence) is as follows:

(SEQ ID NO: 11)
ILGRSETQECLFFNANWEKDRTNQTGVEPCYGDKDKRRHCFA
TWKNISGSIEIVKQGCWLDDINCYDRTDCVEKKDSPEVYFCC
CEGNMCNEKFSYFPEM

The nucleic acid sequence encoding human ActRIIa precursor protein is shown below (SEQ ID NO: 12), as follows nucleotides 159-1700 of Genbank Reference Sequence NM_001616.4. The signal sequence is underlined.

(SEQ ID NO: 12)

| | |
|---|---|
| 1 | ATGGGAGCTG CTGCAAAGTT GGCGTTTGCC |
| | GTCTTTCTTA TCTCCTGTTC |
| 51 | TTCAGGTGCT ATACTTGGTA GATCAGAAAC |
| | TCAGGAGTGT CTTTTCTTTA |
| 101 | ATGCTAATTG GGAAAAAGAC AGAACCAATC |
| | AAACTGGTGT TGAACCGTGT |
| 151 | TATGGTGACA AAGATAAACG GCGGCATTGT |
| | TTTGCTACCT GGAAGAATAT |
| 201 | TTCTGGTTCC ATTGAAATAG TGAAACAAGG |
| | TTGTTGGCTG GATGATATCA |
| 251 | ACTGCTATGA CAGGACTGAT TGTGTAGAAA |
| | AAAAAGACAG CCCTGAAGTA |
| 301 | TATTTTTGTT GCTGTGAGGG CAATATGTGT |
| | AATGAAAAGT TTTCTTATTT |
| 351 | TCCGGAGATG GAAGTCACAC AGCCCACTTC |
| | AAATCCAGTT ACACCTAAGC |
| 401 | CACCCTATTA CAACATCCTG CTCTATTCCT |
| | TGGTGCCACT TATGTTAATT |
| 451 | GCGGGGATTG TCATTTGTGC ATTTTGGGTG |
| | TACAGGCATC ACAAGATGGC |
| 501 | CTACCCTCCT GTACTTGTTC CAACTCAAGA |
| | CCCAGGACCA CCCCCACCTT |
| 551 | CTCCATTACT AGGTTTGAAA CCACTGCAGT |
| | TATTAGAAGT GAAAGCAAGG |
| 601 | GGAAGATTTG GTTGTGTCTG GAAAGCCCAG |
| | TTGCTTAACG AATATGTGGC |
| 651 | TGTCAAAATA TTTCCAATAC AGGACAAACA |
| | GTCATGGCAA AATGAATACG |
| 701 | AAGTCTACAG TTTGCCTGGA ATGAAGCATG |
| | AGAACATATT ACAGTTCATT |
| 751 | GGTGCAGAAA AACGAGGCAC CAGTGTTGAT |
| | GTGGATCTTT GGCTGATCAC |
| 801 | AGCATTTCAT GAAAAGGGTT CACTATCAGA |
| | CTTTCTTAAG GCTAATGTGG |
| 851 | TCTCTTGGAA TGAACTGTGT CATATTGCAG |
| | AAACCATGGC TAGAGGATTG |
| 901 | GCATATTTAC ATGAGGATAT ACCTGGCCTA |
| | AAAGATGGCC ACAAACCTGC |
| 951 | CATATCTCAC AGGGACATCA AAAGTAAAAA |
| | TGTGCTGTTG AAAAACAACC |
| 1001 | TGACAGCTTG CATTGCTGAC TTTGGGTTGG |
| | CCTTAAAATT TGAGGCTGGC |
| 1051 | AAGTCTGCAG GCGATACCCA TGGACAGGTT |
| | GGTACCCGGA GGTACATGGC |
| 1101 | TCCAGAGGTA TTAGAGGGTG CTATAAACTT |
| | CCAAAGGGAT GCATTTTTGA |
| 1151 | GGATAGATAT GTATGCCATG GGATTAGTCC |
| | TATGGGAACT GGCTTCTCGC |
| 1201 | TGTACTGCTG CAGATGGACC TGTAGATGAA |
| | TACATGTTGC CATTTGAGGA |
| 1251 | GGAAATTGGC CAGCATCCAT CTCTTGAAGA |
| | CATGCAGGAA GTTGTTGTGC |
| 1301 | ATAAAAAAAA GAGGCCTGTT TTAAGAGATT |
| | ATTGGCAGAA ACATGCTGGA |
| 1351 | ATGGCAATGC TCTGTGAAAC CATTGAAGAA |
| | TGTTGGGATC ACGACGCAGA |
| 1401 | AGCCAGGTTA TCAGCTGGAT GTGTAGGTGA |
| | AAGAATTACC CAGATGCAGA |
| 1451 | GACTAACAAA TATTATTACC ACAGAGGACA |
| | TTGTAACAGT GGTCACAATG |
| 1501 | GTGACAAATG TTGACTTTCC TCCCAAAGAA |
| | TCTAGTCTA |

The nucleic acid sequence encoding processed soluble (extracellular) human ActRIIa protein is as follows:

| (SEQ ID NO: 13) | |
|---|---|
| | ATACTTGGTA GATCAGAAAC TCAGGAGTGT |
| | CTTTTCTTTA ATGCTAATTG |
| 51 | GGAAAAAGAC AGAACCAATC AAACTGGTGT |
| | TGAACCGTGT TATGGTGACA |
| 101 | AAGATAAACG GCGGCATTGT TTTGCTACCT |
| | GGAAGAATAT TTCTGGTTCC |
| 151 | ATTGAAATAG TGAAACAAGG TTGTTGGCTG |
| | GATGATATCA ACTGCTATGA |
| 201 | CAGGACTGAT TGTGTAGAAA AAAAAGACAG |
| | CCCTGAAGTA TATTTTTGTT |
| 251 | GCTGTGAGGG CAATATGTGT AATGAAAAGT |
| | TTTCTTATTT TCCGGAGATG |
| 301 | GAAGTCACAC AGCCCACTTC AAATCCAGTT |
| | ACACCTAAGC CACCC |

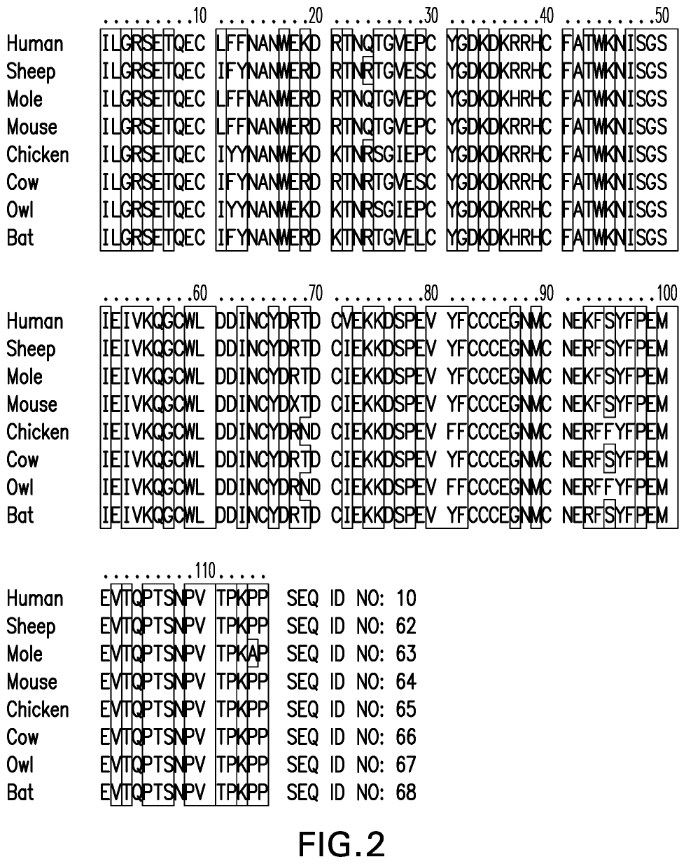

ActRIIa is well-conserved among vertebrates, with large stretches of the extracellular domain completely conserved. For example, FIG. 2 depicts a multi-sequence alignment of a human ActRIIa extracellular domain compared to various ActRIIa orthologs. Many of the ligands that bind to ActRIIa are also highly conserved. Accordingly, from these alignments, it is possible to predict key amino acid positions within the ligand-binding domain that are important for normal ActRIIa-ligand binding activities as well as to predict amino acid positions that are likely to be tolerant to substitution without significantly altering normal ActRIIa-ligand binding activities. Therefore, an active, human ActRIIa variant protein useful in accordance with the presently disclosed methods may include one or more amino acids at corresponding positions from the sequence of another vertebrate ActRIIa, or may include a residue that is similar to that in the human or other vertebrate sequences.

Without meaning to be limiting, the following examples illustrate this approach to defining an active ActRIIa variant. As illustrated in FIG. 2, F13 in the human extracellular domain is Y in Ovis aries (SEQ ID NO: 62), Gallus gallus (SEQ ID NO: 65), Bos Taurus (SEQ ID NO: 66), Tyto alba (SEQ ID NO: 67), and Myotis davidii (SEQ ID NO: 68) ActRIIa, indicating that aromatic residues are tolerated at this position, including F, W, and Y. Q24 in the human extracellular domain is R in Bos Taurus ActRIIa, indicating that charged residues will be tolerated at this position, including D, R, K, H, and E, S95 in the human extracellular domain is F in Gallus gallus and Tyto alba ActRIIa, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRIIa, indicating that acidic residues are tolerated at this position, including D and E. P29 in the human extracellular domain is relatively poorly conserved, appearing as S in Ovis aries ActRIIa and L in Myotis davidii ActRIIa, thus essentially any amino acid should be tolerated at this position.

Moreover, as discussed above, ActRIIa proteins have been characterized in the art in terms of structural/functional characteristics, particularly with respect to ligand binding (Attisano et al. (1992) Cell 68(1):97-108; Greenwald et al. (1999) Nature Structural Biology 6(1): 18-22; Allendorph et al. (2006) PNAS 103(20:7643-7648; Thompson et al. (2003) The EMBO Journal 22(7): 1555-1566; as well as U.S. Pat. Nos. 7,709,605, 7,612,041, and 7,842,663). In addition to the teachings herein, these references provide amply guidance for how to generate ActRIIa variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor (Greenwald et al. (1999) Nat Struct Biol 6:18-22; and Hinck (2012) FEBS Lett 586:1860-1870). Accordingly, the core ligand-binding domains of human ActRIIa, as demarcated by the outermost of these conserved cysteines, corresponds to positions 30-110 of SEQ ID NO: 9 (ActRIIa precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIa extracellular domains truncations include SEQ ID NOs: 10 and 11.

Accordingly, a general formula for an active portion (e.g., ligand binding) of ActRIIa is a protein that comprises, consists essentially of, or consists of amino acids 30-110 of SEQ ID NO: 9. Therefore ActRIIa proteins may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIa beginning at a residue corresponding to any one of amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 9. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 9, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 9. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 9. Thus, in some embodiments, an ActRIIa protein may comprise, consists essentially of, or consist of a protein that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9. Optionally, ActRIIa proteins comprise a protein that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9, and comprising no more than 1, 2, 5, 10 or 15 conservative amino acid changes in the ligand-binding pocket.

In certain embodiments, the disclosure relates to GDF/BMP antagonists (inhibitors) that comprise an ActRIIa protein, which includes fragments, functional variants, and modified forms thereof as well as uses thereof (e.g., increasing an immune response in a patient in need thereof and treating cancer). Preferably, ActRIIa proteins are soluble (e.g., an extracellular domain of ActRIIa). In some embodiments, ActRIIa proteins inhibit (e.g., Smad signaling) of one or more GDF/BMP ligands (e.g., GDF11, GDF8, activin (activin A, activin B, activin AB, activin C, activin E) BMP6, GDF3, BMP15, and/or BMP10). In some embodiments, ActRIIa proteins bind to one or more GDF/BMP ligands (e.g., GDF11, GDF8, activin (activin A, activin B, activin AB, activin C, activin E) BMP6, GDF3, BMP15, and/or BMP10). In some embodiments, ActRIIa protein of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIa beginning at a residue corresponding to amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 9. In some embodiments, ActRIIa proteins comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 9. In certain embodiments, ActRIIa proteins comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 9.

In certain embodiments the extracellular domain (ECD) of the human activin receptor type-IIA (ActRIIa) proteins or derivatives thereof are linked to a constant domain of an immunoglobulin, such as human IgG1 Fc domain. In some embodiments, the ActRIIa protein is a fusion protein comprising an ActRIIa domain and one or more protein domains heterologous to ActRIIa. In some embodiments, the ActRIIa protein is a fusion protein comprising an Fc domain of an immunoglobulin. In some embodiments, the Fc domain of the immunoglobulin is an Fc domain of an IgG1 immunoglobulin. In some embodiments, the ActRIIa fusion protein further comprises a linker domain positioned between the ActRIIa protein domain and the one or more heterologous domains (e.g., an Fc immunoglobulin domain). In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ActRIIa fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 32. In some embodiments, the ActRIIa fusion protein comprises the amino acid sequence of SEQ ID NO: 32. In some embodiments, the ActRIIa fusion protein consists of the amino acid sequence of SEQ ID NO: 32. In some embodiments, the ActRIIa fusion protein consists of the amino acid sequence of SEQ ID NO: 41. In some embodiments, the ActRIIa fusion protein consists of the amino acid sequence of SEQ ID NO: 32 or 41.

In some embodiments, ActRIIa fusion proteins comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, 11, 32, 36, and 39.

In some embodiments, the ActRIIa fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 32. In some embodiments, the ActRIIa fusion protein comprises the amino acid sequence of SEQ ID NO: 32. In some embodiments, the ActRIIa fusion protein consists of the amino acid sequence of SEQ ID NO: 32. In some embodiments, the ActRIIa fusion protein is part of a homodimer protein complex. In some embodiments, the ActRIIa fusion protein is glycosylated. In some embodiments, the ActRIIa fusion protein has a glycosylation pattern obtainable by expression in a Chinese hamster ovary cell.

In some alternative embodiments, the ActRIIa fusion protein (e.g., SEQ ID NO: 32) may lack the C-terminal lysine. In some embodiments, the ActRIIa fusion protein lacking the C-terminal lysine is SEQ ID NO: 41. For example, in certain embodiments of the pharmaceutical formulations described herein, the ActRIIa fusion protein may be comprised of 1-100% of SEQ ID NO: 32. For example, in certain embodiments of the pharmaceutical formulations described herein, the ActRIIa fusion protein is comprised of 1-100% of SEQ ID NO: 41.

In certain embodiments, the pharmaceutical formulations described herein contain a mixture of SEQ ID NO: 32 and a variant of SEQ ID NO:32 lacking the C-terminal lysine residue (SEQ ID NO: 41). In certain embodiments, the pharmaceutical formulations described herein contain a mixture of SEQ ID NO: 32 and a variant of SEQ ID NO:32 lacking the C-terminal lysine residue (SEQ ID NO: 41) wherein the mixture contains 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:32.

In certain embodiments, the pharmaceutical formulations described herein contain a mixture of SEQ ID NO: 32 and a variant of SEQ ID NO:32 lacking the C-terminal lysine residue (SEQ ID NO: 41) wherein the mixture contains about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:32.

In certain embodiments, the pharmaceutical formulations described herein contain a mixture of SEQ ID NO: 32 and a variant of SEQ ID NO:32 lacking the C-terminal lysine residue (SEQ ID NO: 41) wherein the mixture contains 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:41.

In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:41.

In certain embodiments, the pharmaceutical formulations described herein contain 100% of SEQ ID NO: 32 by weight. In certain embodiments, the pharmaceutical formulations described herein contain 100% of SEQ ID NO: 41 by weight.

In certain aspects, the present disclosure relates to GDF trap proteins (also referred to as “GDF traps”). In some embodiments, GDF traps of the present disclosure are variant ActRIIa proteins (e.g., ActRIIa) that comprise one or more mutations (e.g., amino acid additions, deletions, substitutions, and combinations thereof) in the extracellular domain (also referred to as the ligand-binding domain) of an ActRIIa protein (e.g., a “wild-type” or unmodified ActRIIa protein) such that the variant ActRIIa protein has one or more altered ligand-binding activities than the corresponding wild-type ActRIIa protein. In certain embodiments, GDF trap proteins of the present disclosure retain at least one similar activity as a corresponding wild-type ActRIIa protein. For example, preferable GDF traps bind to and inhibit (e.g., antagonize) the function of GDF11 and/or GDF8. In some embodiments, GDF traps of the present disclosure further bind to and inhibit one or more of ligand of the GDF/BMP. Accordingly, the present disclosure provides GDF trap proteins that have an altered binding specificity for one or more ActRIIa ligands.

To illustrate, one or more mutations may be selected that increase the selectivity of the altered ligand-binding domain for GDF11 and/or GDF8 over one or more ActRIIa-binding ligands such as activins, particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

### Methods of Use

In certain aspects, the disclosure provides a method of treating pulmonary arterial hypertension (PAH), comprising administering a pharmaceutical formulation described herein to a patient in need thereof.

In certain aspects, the disclosure provides a method of treating pulmonary arterial hypertension (PAH) in a patient in need thereof, comprising reconstituting a lyophilized pharmaceutical formulation described herein to create a reconstituted formulation and administering the reconstituted formulation to the patient.

In certain aspects, the disclosure provides a pharmaceutical formulation for the treatment of PAH in a subject in need thereof comprising reconstituting a lyophilized pharmaceutical formulation comprising a human ActRIIa fusion protein linked to a constant domain of an immunoglobulin and administering the reconstituted formulation to the subject, wherein the dosing regimen comprises: 1) administering an initial dose of 0.3 mg/kg; 2) monitoring a subject's response; and 3) administering a subsequent dose of 0.7 mg/kg; and wherein the subject is administered the subsequent dose every three weeks. In some embodiments, the subsequent dose is modified based on the subject's response.

In certain aspects, the disclosure provides a method of treating pulmonary arterial hypertension (PAH), comprising administering a pharmaceutical formulation described herein to a patient in need thereof, wherein administration of the pharmaceutical formulation results in a change in one or more of the following hemodynamic or functional parameters: a reduction in pulmonary vascular resistance (PVR); an increase in 6-minute walk distance (6 MWD); a decrease of the N-terminal pro B-type natriuretic peptide (NT-proBNP) levels; the prevention or reduction of pulmonary hypertension Functional Class progression as recognized by the World Health Organization (WHO); the promotion or increasing of pulmonary hypertension Functional Class regression as recognized by the WHO; an improvement in right ventricular function; an improvement in pulmonary artery pressure; and/or an improvement in mean right atrial pressure.

In certain aspects, the disclosure provides a method of treating pulmonary arterial hypertension (PAH), comprising administering a pharmaceutical formulation described herein to a patient in need thereof, wherein administration of the pharmaceutical formulation results in increased exercise capacity, provide clinical improvement, improve WHO functional class (FC), and delay disease progression, including to reduce the risk of death and hospitalization for PAH.

In certain aspects, the disclosure provides a method of treating, preventing, or reducing the progression rate and/or severity of one or more complications of pulmonary arterial hypertension, comprising administering to a patient in need thereof a pharmaceutical formulation described herein, wherein administration of said formulation results in a change in one or more of the following hemodynamic or functional parameters: a reduction in pulmonary vascular resistance (PVR); an increase in 6-minute walk distance (6 MWD); a decrease of the N-terminal pro B-type natriuretic peptide (NT-proBNP) levels; the prevention or reduction in pulmonary hypertension Functional Class progression as recognized by the World Health Organization (WHO); the promotion or increase of pulmonary hypertension Functional Class regression as recognized by the WHO; an improvement in right ventricular function; an improvement in pulmonary artery pressure; and/or an improvement in mean right atrial pressure. In some embodiments, the one or more complications of pulmonary arterial hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis.

In some embodiments, administration of the pharmaceutical formulations described herein reduce the PVR in the patient. In some embodiments, administration of the pharmaceutical formulations described herein reduce the PVR in the patient by at least 10% (e.g., 10%, 15%, 20%, 25%, 30%, 35%, 40%, 45%, or at least 50%). In some embodiments, administration of the pharmaceutical formulations described herein reduce the patient's PVR by at least 20%. In some embodiments, the reduction in PVR is a result of decreased mean pulmonary artery pressure. In some embodiments, administration of the pharmaceutical formulations described herein increase the patient's 6-minute walk distance. In some embodiments, administration of the pharmaceutical formulations described herein increase the patient's 6-minute walk distance by at least 10 meters (e.g., at least 10, 20, 30, 40, 50, 60, 70, 80, 90, 100, 125, 150, 175, 200, 250, 300, or more than 400 meters). In some embodiments, administration of the pharmaceutical formulations described herein increase the patient's 6-minute walk distance by at least 30 meters. In some embodiments, the administration of the pharmaceutical formulations described herein decrease NT-proBNP levels in the patient. In some embodiments, administration of the pharmaceutical formulations described herein decrease NT-proBNP levels in the patient by at least 10% (e.g., 10%, 15%, 20%, 25%, 30%, 35%, 40%, 45%, 50%, 55%, 60%, 65%, 70%, 75%, or at least 80%). In some embodiments, administration of the pharmaceutical formulations described herein decrease NT-proBNP levels in the patient by at least 30%. In some embodiments, administration of the pharmaceutical formulations described herein decrease NT-proBNP levels to normal levels. In some embodiments, the normal level of NT-proBNP is <100 μg/ml.

In some embodiments, administration of the pharmaceutical formulations described herein prevent or reduce pulmonary hypertension Functional Class progression as recognized by the WHO. In some embodiments, administration of the pharmaceutical formulations described herein prevent or reduce pulmonary hypertension Functional Class progression from Functional Class I to Class II pulmonary hypertension as recognized by the WHO. In some embodiments, administration of the pharmaceutical formulations described herein prevent or reduce pulmonary hypertension Functional Class progression from Functional Class II to Class III pulmonary hypertension as recognized by the WHO. In some embodiments, administration of the pharmaceutical formulations described herein prevent or reduce pulmonary hypertension Functional Class progression from Functional Class III to Class IV pulmonary hypertension as recognized by the WHO. In some embodiments, administration of the pharmaceutical formulations described herein promote or increase pulmonary hypertension Functional Class regression as recognized by the WHO. In some embodiments, administration of the pharmaceutical formulations described herein promote or increase pulmonary hypertension Functional Class regression from Class IV to Class III pulmonary hypertension as recognized by the WHO. In some embodiments, administration of the pharmaceutical formulations described herein promote or increase pulmonary hypertension Functional Class regression from Class III to Class II pulmonary hypertension as recognized by the WHO. In some embodiments, administration of the pharmaceutical formulations described herein promote or increase pulmonary hypertension Functional Class regression from Class II to Class I pulmonary hypertension as recognized by the WHO.

In some embodiments, administration of the pharmaceutical formulations described herein improve right ventricular function in the patient. In some embodiments, the improvement in right ventricular function is due to an increase in right ventricular fractional area change. In some embodiments, the improvement in right ventricular function is due to a decrease in right ventricular hypertrophy. In some embodiments, the improvement in right ventricular function is due to an increase in ejection fraction. In some embodiments, the improvement in right ventricular function is due to an increase in right ventricular fractional area change and ejection fraction.

In some embodiments, administration of the pharmaceutical formulations described herein improve the pulmonary artery pressure in the patient. In some embodiments, the improvement in pulmonary artery pressure is a reduction in the mean pulmonary artery pressure (mPAP). In some embodiments, administration of the pharmaceutical formulations described herein reduce the mPAP in the patient by at least 10% (e.g., 10%, 15%, 20%, 25%, 30%, 35%, 40%, 45%, or at least 50%). In some embodiments, administration of the pharmaceutical formulations described herein reduce the mPAP by at least 3 mmHg (e.g., at least 3, 5, 7, 10, 12, 15, 20, or 25 mmHg) in the patient. In some embodiments, administration of the pharmaceutical formulations described herein improve the mean right atrial pressure (mRAP) in the patient. In some embodiments, the improvement in the mRAP is a reduction in the mRAP. In some embodiments, administration of the pharmaceutical formulations described herein reduces the mRAP in the patient by at least 10% (e.g., 10%, 15%, 20%, 25%, 30%, 35%, 40%, 45%, or at least 50%). In some embodiments, administration of the pharmaceutical formulations described herein reduce the mRAP by at least 1 mmHg (e.g., at least 1, 2, 3, 4, 5, 6, 7, 8, 9, 10, 11, 12, 13, 14, or 15 mmHg) in the patient.

In some embodiments, the patient has a pulmonary vascular resistance (PVR) greater than or equal to 3 Wood Units. In some embodiments, the patient has a 6-minute walk distance from 150 to 550 meters. In some embodiments, the patient has elevated NT-proBNP levels as compared to a healthy patient. In some embodiments, the patient has a NT-proBNP level of at least 100 μg/mL (e.g., 100, 150, 200, 300, 400, 500, 1000, 3000, 5000, 10,000, 15,000, or 20,000 μg/mL). In some embodiments, the patient has elevated brain natriuretic peptide (BNP) levels as compared to a healthy patient. In some embodiments, the patient has a BNP level of at least 100 μg/mL (e.g., 100, 150, 200, 300, 400, 500, 1000, 3000, 5000, 10,000, 15,000, or 20,000 μg/mL). In some embodiments, administration of the pharmaceutical formulations described herein decrease BNP levels in the patient by at least 10% (e.g., 10%, 15%, 20%, 25%, 30%, 35%, 40%, 45%, 50%, 55%, 60%, 65%, 70%, 75%, or at least 80%). In some embodiments, administration of the pharmaceutical formulations described herein decrease BNP levels to normal levels (i.e., <100 μg/ml). In some embodiments, the patient has a mean pulmonary artery pressure (mPAP) selected from the group consisting of: an mPAP of at least 20 mmHg; an mPAP of at least 25 mmHg; an mPAP of at least 30 mmHg; an mPAP of at least 35 mmHg; an mPAP of at least 40) mmHg; an mPAP of at least 45 mmHg; and an mPAP of at least 50 mmHg. In some embodiments, the patient has a mean right atrial pressure (mRAP) selected from the group consisting of: an mRAP of at least 5 mmHg; an mRAP of at least 6 mmHg; an mRAP of at least 8 mmHg; an mRAP of at least 10 mmHg; an mRAP of at least 12 mmHg; an mRAP of at least 14 mmHg; and an mRAP of at least 16 mmHg.

In some embodiments, the PAH is idiopathic pulmonary arterial hypertension (PAH). In some embodiments, the PAH is heritable PAH. In some embodiments, the PAH is drug- or toxin-induced PAH. In some embodiments, the PAH is PAH associated with simple, congenital systemic-to-pulmonary shunts at least 1 year following shunt repair. In some embodiments, the patient has Functional Class II or Class III pulmonary hypertension in accordance with the World Health Organization's functional classification system for pulmonary hypertension. In some embodiments, the patient has Functional Class I, Class II, Class III, or Class IV pulmonary hypertension as recognized by the World Health Organization. In some embodiments, the patient has Functional Class I, Class II, Class III, or Class IV pulmonary hypertension in accordance with the World Health Organization's functional classification system for pulmonary hypertension. In some embodiments, the patient has Functional Class IV pulmonary hypertension in accordance with the World Health Organization's functional classification system for pulmonary hypertension. In some embodiments, administration of the pharmaceutical formulations described herein increase transplant free survival in the patient. In some embodiments, administration of the pharmaceutical formulations described herein increase transplant free survival in the patient by at least 10% (e.g., 10%, 15%, 20%, 25%, 30%, 35%, 40%, 45%, or at least 50%). In some embodiments, administration of the pharmaceutical formulations described herein decrease right ventricular hypertrophy in the patient. In some embodiments, administration of the pharmaceutical formulations described herein decrease right ventricular hypertrophy in the patient by at least 10% (e.g., 10%, 15%, 20%, 25%, 30%, 35%, 40%, 45%, or at least 50%). In some embodiments, administration of the pharmaceutical formulations described herein decrease smooth muscle hypertrophy in the patient. In some embodiments, administration of the pharmaceutical formulations described herein decrease smooth muscle hypertrophy in the patient by at least 10% (e.g., 10%, 15%, 20%, 25%, 30%, 35%, 40%, 45%, or at least 50%). In some embodiments, administration of the pharmaceutical formulations described herein decrease pulmonary arteriole muscularity in the patient. In some embodiments, administration of the pharmaceutical formulations described herein decrease pulmonary arteriole muscularity in the patient by at least 10% (e.g., 10%, 15%, 20%, 25%, 30%, 35%, 40%, 45%, or at least 50%).

In some embodiments, administration of the pharmaceutical formulations described herein improve exercise capacity of the patient. In some embodiments, administration of the pharmaceutical formulations described herein decrease the patient's Borg dyspnea index (BDI). In some embodiments, administration of the pharmaceutical formulations described herein reduce the patient's BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points). In some embodiments, the patient has decreased renal function. In some embodiments, administration of the pharmaceutical formulations described herein further improve renal function. In some embodiments, administration of the pharmaceutical formulations described herein delay clinical worsening of pulmonary arterial hypertension. In some embodiments, administration of the pharmaceutical formulations described herein delay clinical worsening of pulmonary arterial hypertension in accordance with the World Health Organization's functional classification system for pulmonary hypertension. In some embodiments, administration of the pharmaceutical formulations described herein reduce the risk of hospitalization for one or more complications associated with pulmonary arterial hypertension. In some embodiments, administration of the pharmaceutical formulations described herein reduce the risk of morbidity for one or more complications associated with pulmonary arterial hypertension. In some embodiments, the morbidity comprises a change in one or more of the following: increased need for a lung and/or heart transplant; need to initiate rescue therapy with a known treatment for PAH; need to increase prostacyclin by at least 10%; need for atrial septostomy; PAH-specific hospitalization for at least 24 hours; and deterioration of PAH. In some embodiments, the deterioration of PAH comprises a worsening in WHO functional class and a decrease in 6 MWD of at least 15%. In some embodiments, administration of the pharmaceutical formulations described herein reduce the risk of death associated with pulmonary arterial hypertension. In some embodiments, administration of the pharmaceutical formulations described herein reduce the risk of death associated with pulmonary arterial hypertension by at least 10% (e.g., 10%, 15%, 20%, 25%, 30%, 35%, 40%, 45%, or at least 50%). In some embodiments, the patient has a hemoglobin level from >8 and <15 g/dl. In some embodiments, the patient's hemoglobin levels are <18 g/dl.

In certain embodiments, the patient treated in accordance with the methods described herein is female. In certain embodiments, the patient treated in accordance with the methods described herein is male. In certain embodiments, the patient treated in accordance with the methods described herein can be of any age. In certain embodiments, the patient treated in accordance with the methods described herein is less than 18 years old. In a specific embodiment, the patient treated in accordance with the methods described herein is less than 13 years old. In another specific embodiment, the patient treated in accordance with the methods described herein is less than 12, less than 11, less than 10, less than 9, less than 8, less than 7, less than 6, or less than 5 years old. In another specific embodiment, the patient treated in accordance with the methods described herein is 1-3 years old, 3-5 years old, 5-7 years old, 7-9 years old, 9-11 years old, 11-13 years old, 13-15 years old, 15-20 years old, 20-25 years old, 25-30 years old, or greater than 30 years old. In another specific embodiment, the patient treated in accordance with the methods described herein is 30-35 years old, 35-40 years old, 40-45 years old, 45-50 years old, 50-55 years old, 55-60 years old, or greater than 60 years old. In another specific embodiment, the patient treated in accordance with the methods described herein is 18-64 years old, 65-74 years old, or greater than 75 years old.

In certain embodiments, hemoglobin levels in a patient treated in accordance with the dosage forms and methods provided herein are less than 10 g/dL, 9 g/dL, 8 g/dL, or 7 g/dL. In certain embodiments, hemoglobin levels in a patient treated in accordance with the dosage forms and methods provided herein are between 7 g/dL and 7.5 g/dL, between 7.5 g/dL and 8 g/dL, between 8 g/dL and 8.5 g/dL, between 8.5 g/dL and 9.0 g/dL, between 9.0 g/dL and 9.5 g/dL, or between 9.5 g/dL and 10.0 g/dL.

### EXAMPLES

The disclosure now being generally described, it will be more readily understood by reference to the following examples, which are included merely for purposes of illustration of certain embodiments of the present disclosure, and are not intended to limit the disclosure.

The invention now being generally described, it will be more readily understood by reference to the following examples, which are included merely for purposes of illustration of certain embodiments of the invention, and are not intended to limit the invention.

### Example 1 ActRIIa-Fc Fusion Proteins

A soluble ActRIIa fusion protein was constructed that has the extracellular domain of human ActRIIa fused to a human or mouse Fc domain with a minimal linker in between. The constructs are referred to as ActRIIa-hFc and ActRIIa-mFc, respectively.

The following ActRIIa-hFc shown below is sotatercept as purified from CHO cell lines (SEQ ID NO: 32):

ILGRSETQECLFFNANWEKDRTNQTGVEPCYGDKDKRRHCFATWKN
ISGSIEIVKQGCWLDDINCYDRTDCVEKKDSPEVYFCCCEGNMCN
EKFSYFPEMEVTQPTSNPVTPKPPTGGGTHTCPPCPAPELLGGPS
VFLFPPKPKDTLMISRTPEVTCVVVDVSHEDPEVKFNWYVDGVEV
HNAKTKPREEQYNSTYRVVSVLTVLHQDWLNGKEYKCKVSNKALP
VPIEKTISKAKGQPREPQVYTLPPSREEMTKNQVSLTCLVKGFYP
SDIAVEWESNGQPENNYKTTPPVLDSDGSFFLYSKLTVDKSRWQQ
GNVFSCSVMHEALHNHYTQKSLSLSPGK

The ActRIIa-hFc and ActRIIa-mFc proteins were expressed in CHO cell lines. Three different leader sequences were considered:

(i)
Honey bee mellitin (HBML):
(SEQ ID NO: 33)
MKFLVNVALVFMVVYISYIYA
(ii) Tissue plasminogen activator (TPA):
(SEQ ID NO: 34)
MDAMKRGLCCVLLLCGAVFVSP
(iii) Native:
(SEQ ID NO: 35)
MGAAAKLAFAVFLISCSSGA

The selected form employs the TPA leader and has the following unprocessed amino acid sequence:

(SEQ ID NO: 36)
MDAMKRGLCCVLLLCGAVFVSPGAAILGRSETQECLFFNANWEKD
RTNQTGVEPCYGDKDKRRHCFATWKNISGSIEIVKQGCWLDDINC
YDRTDCVEKKDSPEVYFCCCEGNMCNEKFSYFPEMEVTQPTSNPV
TPKPPTGGGTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPVPIEKTISKAKGQPREPQV
YTLPPSREEMTKNQVSLTCLVKGFYPSDIAVEWESNGQPENNYKT
TPPVLDSDGSFFLYSKLTVDKSRWQQGNVFSCSVMHEALHNHYTQ
KSLSLSPGK

This protein is encoded by the following nucleic acid sequence:

(SEQ ID NO: 37)
ATGGATGCAATGAAGAGAGGGCTCTGCTGTGTGCTGCTGCTGTGT
GGAGCAGTCTTCGTTTCGCCCGGCGCCGCTATACTTGGTAGATCA
GAAACTCAGGAGTGTCTTTTTTTAATGCTAATTGGGAAAAAGACA
GAACCAATCAAACTGGTGTTGAACCGTGTTATGGTGACAAAGATA
AACGGCGGCATTGTTTTGCTACCTGGAAGAATATTTCTGGTTCCA
TTGAATAGTGAAACAAGGTTGTTGGCTGGATGATATCAACTGCTA
TGACAGGACTGATTGTGTAGAAAAAAAAGACAGCCCTGAAGTATA
TTTCTGTTGCTGTGAGGGCAATATGTGTAATGAAAAGTTTTCTTA
TTTTCCGGAGATGGAAGTCACACAGCCCACTTCAAATCCAGTTAC
ACCTAAGCCACCCACCGGTGGTGGAACTCACACATGCCCACCGTG
CCCAGCACCTGAACTCCTGGGGGGACCGTCAGTCTTCCTCTTCCC
CCCAAAACCCAAGGACACCCTCATGATCTCCCGGACCCCTGAGGT
CACATGCGTGGTGGTGGACGTGAGCCACGAAGACCCTGAGGTCAA
GTTCAACTGGTACGTGGACGGCGTGGAGGTGCATAATGCCAAGAC
AAAGCCGCGGGAGGAGCAGTACAACAGCACGTACCGTGTGGTCAG
CGTCCTCACCGTCCTGCACCAGGACTGGCTGAATGGCAAGGAGTA
CAAGTGCAAGGTCTCCAACAAAGCCCTCCCAGTCCCCATCGAGAA
AACCATCTCCAAAGCCAAAGGGCAGCCCCGAGAACCACAGGTGTA
CACCCTGCCCCCATCCCGGGAGGAGATGACCAAGAACCAGGTCAG
CCTGACCTGCCTGGTCAAAGGCTTCTATCCCAGCGACATCGCCGT
GGAGTGGGAGAGCAATGGGCAGCCGGAGAACAACTACAAGACCAC
GCCTCCCGTGCTGGACTCCGACGGCTCCTTCTTCCTCTATAGCAA
GCTCACCGTGGACAAGAGCAGGTGGCAGCAGGGGAACGTCTTCTC
ATGCTCCGTGATGCATGAGGCTCTGCACAACCACTACACGCAGAA
GAGCCTCTCCCTGTCTCCGGGTAAATGAGAATTC

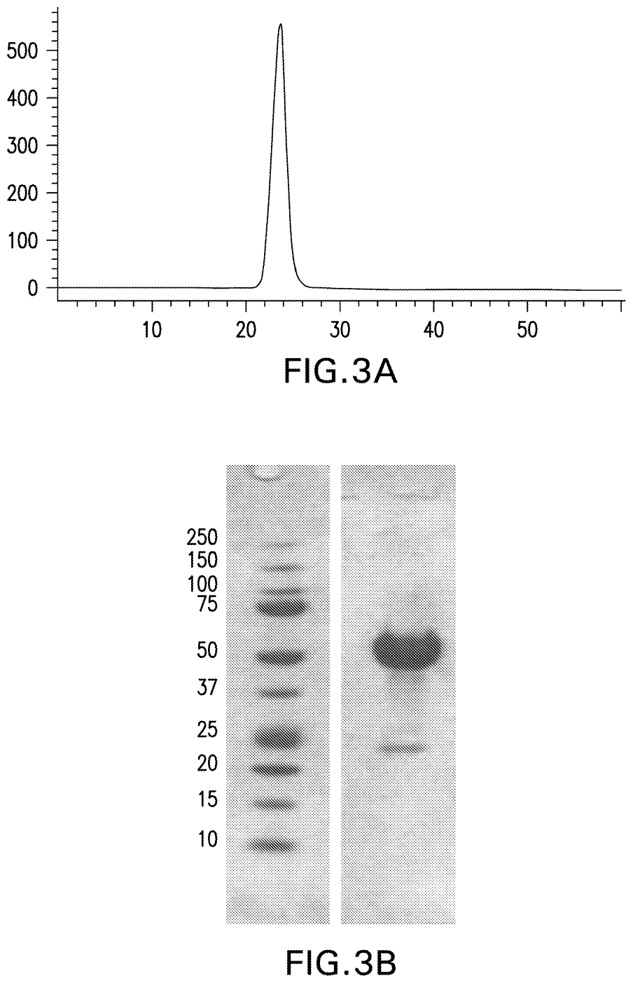

Both ActRIIa-hFc and ActRIIa-mFc were remarkably amenable to recombinant expression. As shown in FIGS. 3A and 3B, the protein was purified as a single, well-defined peak of protein. N-terminal sequencing revealed a single sequence of -ILGRSETQE (SEQ ID NO: 38). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIa-hFc protein was purified to a purity of >98% as determined by size exclusion chromatography and >95% as determined by SDS PAGE.

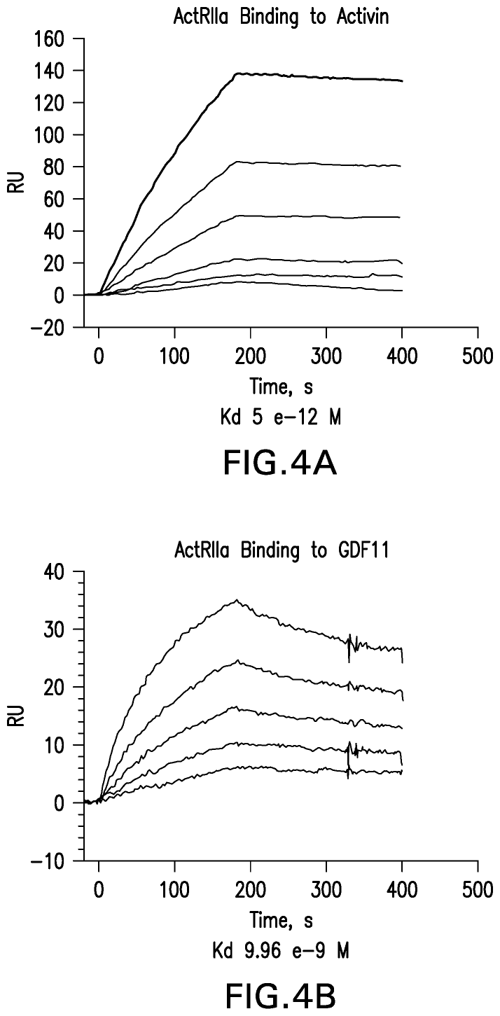

ActRIIa-hFc and ActRIIa-mFc showed a high affinity for ligands. GDF11 or activin A were immobilized on a Biacore™ CM5 chip using standard amine-coupling procedure. ActRIIa-hFc and ActRIIa-mFc proteins were loaded onto the system, and binding was measured. ActRIIa-hFc bound to activin with a dissociation constant (K_{D}) of 5×10⁻¹² and bound to GDF11 with a K_{D} of 9.96×10⁻⁹. See FIGS. 4A and 4B. Using a similar binding assay, ActRIIa-hFc was determined to have high to moderate affinity for other TGF-beta superfamily ligands including, for example, activin B, GDF8, BMP6, and BMP10. ActRIIa-mFc behaved similarly.

The ActRIIa-hFc was very stable in pharmacokinetic studies. Rats were dosed with 1 mg/kg, 3 mg/kg, or 10 mg/kg of ActRIIa-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIa-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2 Characterization of an ActRIIa-hFc Protein

ActRIIa-hFc fusion protein was expressed in stably transfected CHO-DUKX B11 cells from a pAID4 vector (SV40 ori/enhancer, CMV promoter), using a tissue plasminogen leader sequence of SEQ ID NO: 34. The protein, purified as described above in Example 1, had a sequence of SEQ ID NO: 32. The Fc portion is a human IgG1 Fc sequence, as shown in SEQ ID NO: 32. Protein analysis reveals that the ActRIIa-hFc fusion protein is formed as a homodimer with disulfide bonding.

The CHO-cell-expressed material has a higher affinity for activin B ligand than that reported for an ActRIIa-hFc fusion protein expressed in human 293 cells (see, del Re et al. (2004) J Biol Chem. 279(51):53126-53135). Additionally, the use of the TPA leader sequence provided greater production than other leader sequences and, unlike ActRIIa-Fc expressed with a native leader, provided a highly pure N-terminal sequence. Use of the native leader sequence resulted in two major species of ActRIIa-Fc, each having a different N-terminal sequence.

Additional ActRIIa Ligand Traps (ActRIIa-Fc fusion proteins modified so as to reduce the ratio of activin A binding relative to myostatin or GDF11) are described in International Patent Application Publication Nos. WO 2006/012627 and WO 2007/062188, incorporated by reference herein.

### Example 3 Preparation of a Lyophilized ActRIIa-hFc Fusion Protein Formulation

Initially the ActRIIa-hFc fusion protein SEQ ID NO: 32 (sotatercept) was first formulated in phosphate buffered saline as a frozen solution. Based on the subsequent development studies performed, a lyophilized citrate buffer formulation of the ActRIIa-hFc fusion protein SEQ ID NO: 32 was developed containing sucrose and polysorbate 80, to enable a stable formulation with adequate shelf life for commercialization.

Two formulations of the ActRIIa-hFc fusion protein SEQ ID NO: 32, 45 mg/vial and 60 mg/vial were developed. The 45 mg/vial formulation contained 55.0 mg of SEQ ID NO: 32 or a variant of SEQ ID NO:32 lacking the C-terminal lysine residue, 0.48 mg of citric acid monohydrate, 2.56 mg tri-sodium citrate dihydrate, 0.22 mg polysorbate 80, and 88.0 mg sucrose. The 60 mg/ml formulation contained 72.5 mg of SEQ ID NO: 32 or a variant of SEQ ID NO: 32 lacking the C-terminal lysine residue, 0.64 mg of citric acid monohydrate, 3.37 mg tri-sodium citrate dihydrate, 0.29 mg polysorbate 80, and 116.0 mg sucrose. The formulations are identical composition prior to lyophilization and upon reconstitution with sWFI. They differ only in their fill volumes. The 45 mg/vial has a fill volume of 1.1 mL whereas the 60 mg/vial has a fill volume of 1.45 mL. Both formulations are lyophilized and are reconstituted prior to subcutaneous administration. The 45 mg/vial is reconstituted with 1.0 mL sWFI and the 60 mg/vial with 1.3 mL sWFI. The The formulation development of the ActRIIa-hFc fusion protein SEQ ID NO: 32 was guided by the results of various screening studies described below. These studies were focused on the selection of optimal pH, buffer system, protein concentration, and excipients for stabilization of the ActRIIa-hFc fusion protein SEQ ID NO: 32. The screening studies were conducted at accelerated or stressed conditions to observe differences in the stability of the ActRIIa-hFc fusion protein SEQ ID NO: 32 in the test samples.

### Preliminary pH Screening Study: Early Evaluation and Identification of a Suitable Buffer And pH Range for the ActRIIa-hFc Fusion Protein SEQ ID NO: 32 for Optimal Biophysical Stability and Binding Capacity

An initial buffer and pH screen was performed to understand the impact of pH and buffer system on the stability of the ActRIIa-hFc fusion protein SEQ ID NO: 32. In this study the ActRIIa-hFc fusion protein SEQ ID NO: 32 was formulated to a concentration of 5 mg/mL in acetate, citrate, histidine, phosphate, succinate, and tris buffers at 50 mM concentration, with pH levels ranging from 4 to 8. These formulations were filled into Type I glass vials and placed on short-term stability at 5° C., 25° C., and 45° C. for up to 6 months and analyzed for purity by high performance size exclusion chromatography (HP-SEC), thermal stability using differential scanning calorimetry (DSC) and binding activity using surface plasma resonance (Biacore). The data from these analyses is summarized in Table 1.

TABLE 1
Preliminary pH Screen Study 1: Effect of Buffer and pH on
% Monomer Species by HP-SEC, Tml by DSC, Kd by Biacore

| | | Time Point (Months, M); Monomer (%) | | | | | | | Time Point (Months, M); Ka (PM) | | | | | |
|---|---|---|---|---|---|---|---|---|---|---|---|---|---|---|
| Buffer | Tm1 | | 5° C. | | 25° C. | | 45° C. | | | 5° C. | 25° C. | | 45° C. | |
| System | (° C.) | Initial | 3 M | 6 M | 3 M | 6 M | 1 M | 2 M | Initial | 6 M | 2 M | 6 M | 1 M | 2 M |
| Acetate, | 55.6 | 99.7 | 99.9 | 99.7 | 99.8 | 99.8 | 89.4 | 88.4 | 8.4 | 21.1 | 47 | 192 | 127 | 272 |
| pH 4.0 | | | | | | | | | | | | | | |
| Acetate, | 59.7 | 99.7 | 99.9 | 99.7 | 99.8 | 99.8 | 95.3 | 91.7 | 8.0 | 17.1 | 41 | 115 | 138 | 184 |
| pH 4.5 | | | | | | | | | | | | | | |
| Acetate, | 63.1 | 99.8 | 99.9 | 100.0 | 99.8 | 100.0 | 99.4 | 96.0 | 7.5 | 11.3 | 23 | 50.0 | 77.5 | 186 |
| pH 5.0 | | | | | | | | | | | | | | |
| Acetate, | 66.8 | 99.8 | 99.9 | 100.0 | 99.9 | 100.0 | 99.8 | 97.9 | 9.7 | 10.6 | 13 | 23.2 | 41.8 | 53.4 |
| pH 5.5 | | | | | | | | | | | | | | |
| Citrate, pH | 57.9 | 99.8 | 99.9 | 99.8 | 99.9 | 99.8 | 85.2 | 66.2 | 9.2 | 16.4 | 46 | 181 | 251 | 24.9 |
| 4.5 | | | | | | | | | | | | | | |
| Citrate, pH | 64.0 | 99.8 | 99.9 | 100.0 | 99.9 | 99.7 | 98.6 | 94.3 | 9.9 | 13.0 | 29 | 70.0 | 307 | 84.2 |
| 5.0 | | | | | | | | | | | | | | |
| Citrate, pH | 66.2 | 99.8 | 99.9 | 100.0 | 99.9 | 100.0 | 99.8 | 97.8 | 10.7 | 10.1 | 13 | 27.3 | 46.4 | 56.2 |
| 5.5 | | | | | | | | | | | | | | |
| Citrate, pH | 68.5 | 99.8 | 99.9 | 100.0 | 99.9 | 100.0 | 99.8 | 97.2 | 9.4 | 9.7 | 11 | 17.7 | 21.5 | 33.0 |
| 6.0 | | | | | | | | | | | | | | |
| Citrate, pH | 69.4 | 99.8 | 99.9 | 100.0 | 99.9 | 100.0 | 99.1 | 96.7 | 10.0 | 11.5 | 8.6 | 12.4 | 13.4 | 8.3 |
| 6.5 | | | | | | | | | | | | | | |
| Citrate, pH | 69.5 | 99.8 | 99.9 | 100.0 | 99.9 | 98.1 | 99.0 | 93.7 | 9.4 | 8.4 | 7.2 | 13.1 | 11.5 | 18.0 |
| 7.0 | | | | | | | | | | | | | | |
| Histidine, | 62.7 | 99.8 | 99.9 | 100.0 | 99.9 | 100.0 | 99.6 | 96.3 | 10.9 | 10.9 | 14 | 31.3 | 82.4 | 170 |
| pH 5.5 | | | | | | | | | | | | | | |
| Histidine, | 65.8 | 99.8 | 99.9 | 100.0 | 99.9 | 100.0 | 99.8 | 97.6 | 10.4 | 10.5 | 11 | 20.2 | 50.7 | 55.3 |
| pH 6.0 | | | | | | | | | | | | | | |
| Histidine, | 67.5 | 99.8 | 99.9 | 100.0 | 98.8 | 98.3 | 99.9 | 90.0 | 9.3 | 8.8 | 9.3 | 14.9 | 29.2 | 32.0 |
| pH 6.5 | | | | | | | | | | | | | | |
| Histidine, | 67.8 | 99.8 | 99.9 | 100.0 | 99.9 | 98.0 | 99.9 | 88.8 | 8.7 | 8.3 | 10 | 16.0 | 21.0 | 29.3 |
| pH 7.0 | | | | | | | | | | | | | | |
| Histidine, | 67.9 | 99.8 | 99.9 | 100.0 | 99.9 | 95.9 | 100.0 | 87.1 | 10.1 | 9.4 | 9.1 | 15.9 | 19.2 | 26.2 |
| pH 7.4 | | | | | | | | | | | | | | |
| Phosphate, | 69.1 | 99.8 | 99.9 | 100.0 | 99.9 | 100.0 | 99.8 | 97.7 | 8.0 | 9.8 | 10 | 19.3 | 34.7 | 41.8 |
| pH 5.8 | | | | | | | | | | | | | | |
| Phosphate, | 69.4 | 99.8 | 99.9 | 100.0 | 99.9 | 100.0 | 99.8 | 97.0 | 9.0 | 10.7 | 11 | 15.8 | 20.6 | 27.7 |
| pH 6.3 | | | | | | | | | | | | | | |
| Phosphate, | 69.9 | 99.8 | 99.9 | 99.6 | 99.9 | 96.7 | 98.3 | 94.3 | 10.0 | 8.6 | 6.5 | 11.9 | 15.5 | 16.7 |
| pH 6.8 | | | | | | | | | | | | | | |
| Phosphate, | 70.2 | 99.8 | 99.8 | 100.0 | 99.9 | 95.8 | 92.3 | 87.9 | 10.0 | 10.0 | 7.3 | 8.6 | 13.8 | 20.7 |
| pH 7.3 | | | | | | | | | | | | | | |
| Phosphate, | 70.0 | 99.8 | 99.9 | 100.0 | 99.5 | 90.4 | 87.7 | 63.7 | 9.0 | 9.4 | 7.7 | 12.9 | 17.7 | 18.0 |
| pH 7.8 | | | | | | | | | | | | | | |
| Succinate, | 59.4 | 99.8 | 99.8 | 99.7 | 99.8 | 99.7 | 95.2 | 91.5 | 9.5 | 18.3 | 42 | 114.0 | 108 | 65.8 |
| pH 4.5 | | | | | | | | | | | | | | |
| Succinate, | 64.0 | 99.8 | 99.8 | 100.0 | 99.9 | 99.8 | 99.5 | 95.4 | 12.1 | 13.8 | 25 | 48.9 | 73.4 | 106 |
| pH 5.0 | | | | | | | | | | | | | | |
| Succinate, | 67.1 | 99.8 | 99.8 | 100.0 | 99.8 | 99.8 | 99.8 | 96.4 | 10.5 | 10.8 | 15 | 25.0 | 31.6 | 65.6 |
| pH 5.5 | | | | | | | | | | | | | | |
| Succinate, | 68.6 | 99.8 | 99.9 | 100.0 | 99.8 | 99.8 | 99.7 | 95.9 | 11.5 | 5.8 | 8.4 | 12.0 | 17.7 | 5.7 |
| pH 6.0 | | | | | | | | | | | | | | |
| Succinate, | 69.5 | 99.8 | 99.9 | 100.0 | 99.7 | 99.7 | 98.8 | 96.2 | 9.8 | 10.4 | 7.6 | 10.2 | 10.9 | 14.3 |
| pH 6.5 | | | | | | | | | | | | | | |
| Tris, pH | 68.9 | 99.8 | 99.9 | 100.0 | 99.9 | 97.3 | 99.8 | 97.1 | 9.7 | 8.4 | 6.5 | 11.3 | 12.6 | 25.3 |
| 7.0 | | | | | | | | | | | | | | |
| Tris, pH | 69.2 | 99.8 | 99.9 | 100.0 | 99.9 | 97.1 | 98.1 | 96.4 | 11.2 | 8.1 | 10 | 9.3 | 11.1 | 24.5 |
| 7.3 | | | | | | | | | | | | | | |
| Tris, pH | 69.1 | 99.8 | 99.9 | 99.3 | 99.8 | 95.8 | 99.2 | 95.3 | 10.6 | 10.7 | 8.1 | 10.8 | 15.5 | 19.8 |
| 7.5 | | | | | | | | | | | | | | |
| Tris, pH | 69.0 | 99.8 | 99.9 | 100 | 99.9 | 95.7 | 99.0 | 94.6 | 10.5 | 6.8 | 9.8 | 12.3 | 10.9 | 21.3 |
| 7.8 | | | | | | | | | | | | | | |
| Tris, pH | 69.0 | 99.8 | 99.9 | 100 | 99.8 | 95.7 | 98.9 | 94.0 | 9.6 | 7.4 | 10 | 12.7 | 10.4 | 22.6 |
| 8.0 | | | | | | | | | | | | | | |

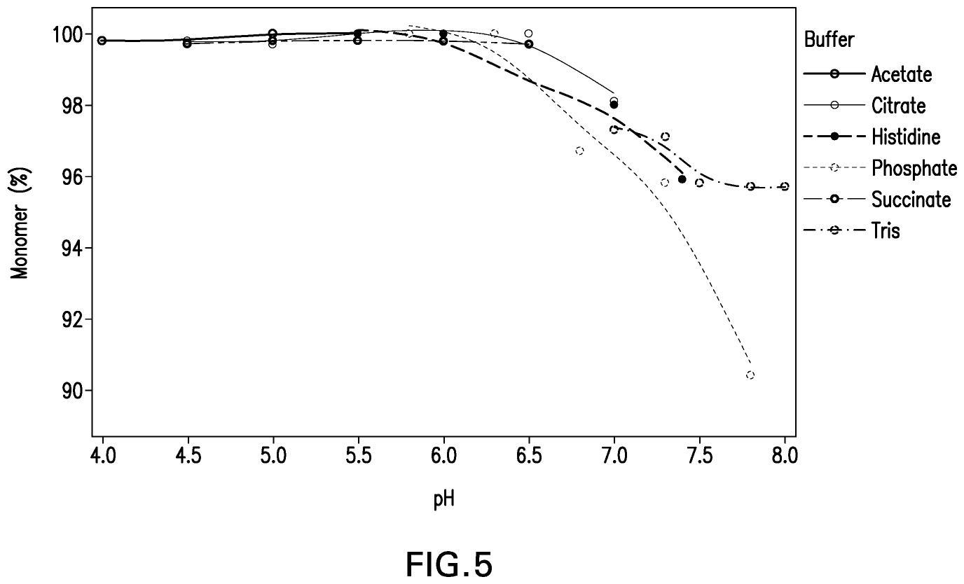

Observation of changes in % monomer species (purity) by HP-SEC revealed that there was no significant change in purity after 6 months at 5° C. with all formulations retaining ≥99.0% monomer. After 6 months at 25° C. there was a significant decrease in % monomer for formulations with a pH ≥6.5, whereas there were no significant changes for any formulation below pH 6.5 as shown in FIG. 5. After 2 months at 45° C. there was a similar decrease in % monomer for formulations with pH ≥6.5, but there was also decreasing % monomer correlated with lower pH<5.5.

Based on these observations at accelerated (25° C.) and stressed (45° C.) conditions, ActRIIa-hFc fusion protein SEQ ID NO: 32 in citrate & succinate buffer in the pH range of 5.5-6.5 had the greatest stabilizing effect. Histidine at pH 5.5-6.0 also had similar stability to citrate and succinate at lower pH and less stable at higher pH.

Binding affinity is typically measured by the equilibrium dissociation constant (Kd), which is used to evaluate and rank order strengths of bimolecular interactions. In this case the interaction or binding affinity of ActRIIa-hFc fusion protein SEQ ID NO: 32 with its target activin by Biacore was used as a measure of product stability. The smaller the Kd value, the greater the binding affinity of the ligand for its target.

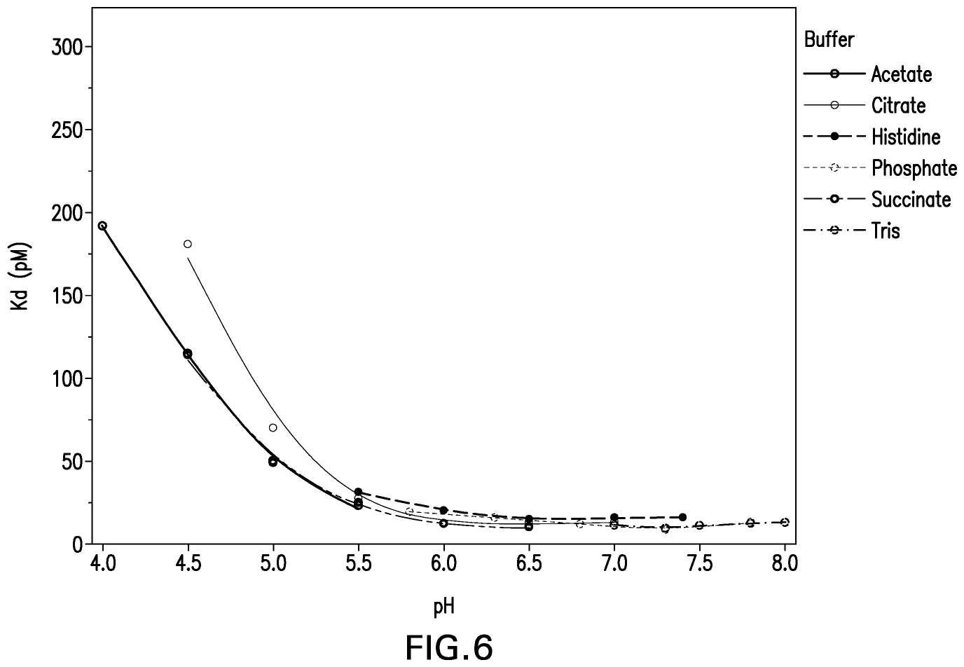

There was no significant change in the equilibrium dissociation constant (Kd) of SEQ ID NO: 32 after 6 months at 5° C. After 6 months at 25° C. for formulations with a pH<5.5 there was a significant decrease in binding as shown by a steep increase in Kd values in FIG. 6. Formulations with pH<6.0 had a decrease in binding after 2 months at 45° C.

Based on these observations, the ActRIIa-hFc fusion protein SEQ ID NO: 32 can retain its binding affinity at pH ≥5.5 in many different buffer systems.

The unfolding temperature of ActRIIa-hFc fusion protein SEQ ID NO: 32 increased consistently with pH, plateauing near 67° C.-70° C. around pH ≥5.5. All buffers at equivalent pH values performed identically except for histidine and tris systems which have significantly lower melting points.

In totality, the results of this experiment suggested that a pH range of 5.5 to 6.5 would be optimal range for SEQ ID NO: 32 in terms of physical stability monitored by HP-SEC and retention of binding affinity by Biacore. Additionally, citrate and succinate buffer systems performed superior to histidine at the higher end of this range. Also, acetate and phosphate buffers were not pursued further in development because of the potential for pH changes due to precipitation during freezing or lyophilization.

Determination of Optimal Buffer Concentration: Evaluation of the Effect of Buffer Concentration with One of the Lead Buffer Systems, Citrate, on Conformational and Colloidal Stability of ActRIIa-hFc Fusion Protein SEQ ID NO: 32

In the initial pH screening study, a variety of buffer systems were evaluated at 50 mM concentration to provide a high buffer capacity. However, this concentration may not be optimal for stabilizing the ActRIIa-hFc fusion protein SEQ ID NO: 32 and is not ideal for subcutaneous injections where higher ionic strength of buffer, especially from citrate, are known to cause injection pain. In this study, the ActRIIa-hFc fusion protein SEQ ID NO: 32 was prepared in a range of citrate buffer concentrations, one of the lead buffer systems from the previous study. Citrate buffer (5-50 mM) at pH 5.8 with 50 mg/mL the ActRIIa-hFc fusion protein SEQ ID NO: 32 and 8% (weight/volume) sucrose were evaluated for the effect of buffer strength on thermal stability using DSC and diffusion interaction parameter (kD) by DLS, which is a measure of a molecule's propensity to self-associate. The results are summarized in Table 2.

TABLE 2
Buffer Concentration Ranging Study: Effect of Buffer
Concentration on the Melting Points and kD

| Citrate (mM) | Tm1 (° C.) | k_{D} (mL/g) |
|---|---|---|
| 5 | 67.9 | 6.7 |
| 10 | 68.8 | 7.4 |
| 25 | 69.0 | 1.4 |
| 50 | 68.8 | −4.5 |

The results of the study showed that the highest dissociation constant (kD) of the ActRIIa-hFc fusion protein SEQ ID NO: 32 was at 10 mM (+6.7 mL/g) and would decrease with higher concentrations up to 50 mM which resulted in a negative kD (−4.5 mL/g). The more positive a molecules kD value indicates stronger repulsion between molecules which is indicative of better colloidal stability while a negative kD value indicate a propensity to self-associate which can lead to aggregation. Additionally, the melting points of the ActRIIa-hFc fusion protein SEQ ID NO: 32 were largely identical and sufficiently high for all buffer concentrations. Based on this data, a 10 mM buffer concentration was chosen for subsequent formulation development.

### Selection of pH of Optimal Stability

A short-term stability study was conducted where the ActRIIa-hFc fusion protein SEQ ID NO: 32 was formulated to a concentration of 5 mg/mL in succinate, citrate, and histidine at 10 mM concentration with pH levels ranging from 5 to 7. These formulations were filled into Type I glass vials and placed on short-term stability at 5° C., 25° C., and 40° C. for up to 3 months. The formulations were analyzed by biophysical methods for purity by high performance size exclusion chromatography (HP-SEC), thermal stability using differential scanning calorimetry (DSC), fragmentation by CE-SDS Non-Reduced, chemical degradation by desialylated iCIEF and pH. The data from these analyses is summarized in Table 3, Table 4, and Table 5 and the following observations were made:

TABLE 3
Selection of pH of Optimal Stability Study: Effect of Buffer and
pH on % HMW, Monomer, and % LMW Species by HP-SEC, Tml by DSC

| | | Time Point (Months, M); | | | | Time Point (Months, M); | | | | Time Point | | | |
|---|---|---|---|---|---|---|---|---|---|---|---|---|---|
| | | HMW (%) | | | | Monomer (%) | | | | (Months, M); | | | |
| Buffer | DSC | | 5° C. | 25° C. | 40° C. | | 5° C. | 25° C. | 40° C. | LMW (%) | 5° C. | 25° C. | 40° C. |
| System | Tml | Initial | 3 M | 3 M | 3 M | Initial | 3 M | 3 M | 3 M | Initial | 3 M | 3 M | 3 M |
| Succinate | 61.2 | 0.7 | 0.5 | 0.5 | N/A | 98.7 | 98.9 | 98.4 | N/A | 0.7 | 0.6 | 1.0 | N/A |
| pH 5.0 | | | | | | | | | | | | | |
| Succinate | 65.3 | 0.5 | 0.5 | 0.5 | 0.7 | 98.9 | 99.0 | 98.7 | 96.1 | 0.7 | 0.6 | 0.9 | 3.1 |
| pH 5.5 | | | | | | | | | | | | | |
| Succinate | 68.1 | 0.5 | 0.4 | 0.4 | 0.5 | 98.9 | 99.0 | 98.7 | 96.3 | 0.6 | 0.5 | 0.9 | 3.3 |
| pH 6.0 | | | | | | | | | | | | | |
| Succinate | 69.7 | 0.5 | 0.5 | 0.4 | 0.4 | 98.9 | 99.0 | 98.6 | 95.5 | 0.6 | 0.6 | 1.0 | 4.1 |
| pH 6.5 | | | | | | | | | | | | | |
| Citrate pH | 61.7 | 0.5 | 0.5 | 0.5 | 1.5 | 98.9 | 99.0 | 98.5 | 95.0 | 0.6 | 0.5 | 1.0 | 3.6 |
| 5.0 | | | | | | | | | | | | | |
| Citrate pH | 65.4 | 0.5 | 0.5 | 0.4 | 0.7 | 98.9 | 99.1 | 98.7 | 96.2 | 0.6 | 0.5 | 0.9 | 3.2 |
| 5.5 | | | | | | | | | | | | | |
| Citrate pH | 68.6 | 0.5 | 0.4 | 0.4 | 0.5 | 98.9 | 99.1 | 98.7 | 96.1 | 0.6 | 0.5 | 0.9 | 3.4 |
| 6.0 | | | | | | | | | | | | | |
| Citrate pH | 70.0 | 0.5 | 0.5 | 0.4 | 0.4 | 99.0 | 99.0 | 98.6 | 95.1 | 0.6 | 0.5 | 1.0 | 4.5 |
| 6.5 | | | | | | | | | | | | | |
| Citrate pH | 70.4 | 0.5 | 0.5 | 0.4 | 0.4 | 98.9 | 99.0 | 98.4 | 93.8 | 0.6 | 0.5 | 1.2 | 5.8 |
| 7.0 | | | | | | | | | | | | | |
| Histidine | 60.9 | 0.5 | 0.5 | 0.4 | 1.1 | 98.9 | 99.1 | 98.7 | 95.6 | 0.6 | 0.5 | 0.9 | 3.3 |
| pH 5.5 | | | | | | | | | | | | | |
| Histidine | 63.7 | 0.5 | 0.4 | 0.3 | 0.5 | 99.0 | 99.1 | 98.8 | 96.3 | 0.6 | 0.5 | 0.9 | 3.1 |
| pH 6.0 | | | | | | | | | | | | | |
| Histidine | 66.3 | 0.5 | 0.4 | 0.3 | 0.3 | 99.0 | 99.1 | 98.7 | 96.4 | 0.6 | 0.5 | 1.0 | 3.3 |
| pH 6.5 | | | | | | | | | | | | | |
| Histidine | 66.9 | 0.5 | 0.4 | 0.3 | 0.3 | 99.0 | 99.0 | 98.7 | 96.1 | 0.6 | 0.6 | 1.0 | 3.6 |
| pH 7.0 | | | | | | | | | | | | | |

TABLE 4
Selection of pH of Optimal Stability Study: Effect of Buffer
and pH on % Minor Peak and % Major Peak Species by CE-SDS NR

| | | Time Point (Months, M); | | | | Time Point (Months, M); | | | |
|---|---|---|---|---|---|---|---|---|---|
| | | Minor Peak (%) | | | | Major Peak (%) | | | |
| Buffer | pH | | 5° C. | 25° C. | 40° C. | | 5° C. | 25° C. | 40° C. |
| System | Initial | Initial | 1 M | 1 M | 1 M | Initial | 1 M | 1 M | 1 M |
| Succinate pH | 5.0 | 1.9 | 2.2 | 3.4 | 6.3 | 98.0 | 97.7 | 96.6 | 93.8 |
| 5.0 | | | | | | | | | |
| Succinate pH | 5.5 | 1.9 | 2.2 | 2.3 | 4.5 | 98.0 | 97.8 | 97.7 | 95.4 |
| 5.5 | | | | | | | | | |
| Succinate pH | 5.9 | 2.0 | 2.8 | 2.4 | 3.2 | 98.0 | 97.1 | 97.6 | 96.8 |
| 6.0 | | | | | | | | | |
| Succinate pH | 6.5 | 2.2 | 2.3 | 2.4 | 3.4 | 97.8 | 97.7 | 97.6 | 96.7 |
| 6.5 | | | | | | | | | |
| Citrate pH 5.0 | 4.9 | 3.5 | 2.2 | 3.4 | 6.9 | 96.5 | 97.9 | 96.6 | 93.0 |
| Citrate pH 5.5 | 5.4 | 3.4 | 2.4 | 3.0 | 3.5 | 96.5 | 97.6 | 97.0 | 96.4 |
| Citrate pH 6.0 | 5.7 | 2.1 | 1.8 | 2.4 | 4.1 | 97.9 | 98.2 | 97.6 | 95.9 |
| Citrate pH 6.5 | 6.3 | 1.9 | 1.8 | 2.1 | 2.9 | 98.1 | 98.1 | 97.9 | 97.0 |
| Citrate pH 7.0 | 6.7 | 1.7 | 2.1 | 2.1 | 3.6 | 98.3 | 97.9 | 97.9 | 96.3 |
| Histidine pH | 5.7 | 1.9 | 2.1 | 2.8 | 4.5 | 98.1 | 97.8 | 97.2 | 95.4 |
| 5.5 | | | | | | | | | |
| Histidine pH | 6.1 | 1.9 | 2.0 | 1.9 | 3.3 | 98.1 | 98.0 | 98.1 | 96.7 |
| 6.0 | | | | | | | | | |
| Histidine pH | 6.6 | 1.8 | 2.0 | 2.1 | 3.2 | 98.3 | 98.0 | 97.8 | 96.8 |
| 6.5 | | | | | | | | | |
| Histidine pH | 6.9 | 2.0 | 2.3 | 2.3 | 3.4 | 98.0 | 97.6 | 97.8 | 96.5 |
| 7.0 | | | | | | | | | |

TABLE 5
Selection of pH of Optimal Stability Study: Effect of Buffer and pH on % Acidic
Species, % Basic Species, and % Total Main Species by Desialylated icIEF

| | Time Point (Months, M); | | | | Time Point (Months, M); | | | | Time Point (Months, M); | | | |
|---|---|---|---|---|---|---|---|---|---|---|---|---|
| | Acidic Species (%) | | | | Total Main Species (%) | | | | Basic Species (%) | | | |
| Buffer | | 5° C. | 25° C. | 40° C. | | 5° C. | 25° C. | 40° C. | | 5° C. | 25° C. | 40° C. |
| System | Initial | 3 M | 3 M | 3 M | Initial | 3 M | 3 M | 3 M | Initial | 3 M | 3 M | 3 M |
| Succinate | 19.6 | 18.5 | 12.3 | DNT | 64.1 | 65.3 | 50.9 | DNT | 16.4 | 16.2 | 36.8 | DNT |
| pH 5.0 | | | | | | | | | | | | |
| Succinate | 17.2 | 17.8 | 15.1 | 13.9 | 65.8 | 67.3 | 60.8 | 45.1 | 17.0 | 15.0 | 24.1 | 41.0 |
| pH 5.5 | | | | | | | | | | | | |
| Succinate | 16.8 | 16.2 | 16.4 | 19.1 | 65.5 | 69.7 | 65.3 | 53.6 | 17.7 | 14.1 | 18.3 | 27.3 |
| pH 6.0 | | | | | | | | | | | | |
| Succinate | 17.3 | 17.3 | 17.9 | DNT | 65.1 | 67.7 | 66.5 | DNT | 17.6 | 15.1 | 15.6 | DNT |
| pH 6.5 | | | | | | | | | | | | |
| Citrate pH | 18.1 | 15.7 | 11.6 | 16.3 | 64.1 | 65.5 | 51.2 | 28.6 | 17.7 | 18.8 | 37.3 | 55.1 |
| 5.0 | | | | | | | | | | | | |
| Citrate pH | 17.7 | 16.6 | 14.9 | 24.0 | 65.2 | 68.4 | 59.8 | 37.5 | 17.1 | 15.0 | 25.3 | 38.4 |
| 5.5 | | | | | | | | | | | | |
| Citrate pH | 17.5 | 16.3 | 15.7 | 22.2 | 64.9 | 69.1 | 65.9 | 52.1 | 17.6 | 14.7 | 18.4 | 25.7 |
| 6.0 | | | | | | | | | | | | |
| Citrate pH | 17.6 | 17.4 | 18.0 | 26.8 | 65.1 | 69.0 | 66.3 | 55.0 | 17.2 | 13.6 | 15.7 | 18.2 |
| 6.5 | | | | | | | | | | | | |
| Citrate pH | 18.2 | 18.7 | 18.1 | 36.0 | 64.9 | 69.1 | 68.0 | 49.6 | 16.9 | 12.3 | 13.8 | 14.4 |
| 7.0 | | | | | | | | | | | | |
| Histidine | 19.1 | 18.9 | 16.0 | 13.7 | 64.2 | 68.0 | 59.4 | 31.4 | 16.7 | 13.1 | 24.6 | 54.9 |
| pH 5.5 | | | | | | | | | | | | |
| Histidine | 17.6 | 17.8 | 16.0 | 13.1 | 65.6 | 69.5 | 64.8 | 47.7 | 16.8 | 12.8 | 19.2 | 39.2 |
| pH 6.0 | | | | | | | | | | | | |
| Histidine | 17.0 | 17.8 | 17.5 | 19.6 | 66.0 | 69.1 | 66.9 | 53.2 | 17.0 | 13.1 | 15.6 | 27.1 |
| pH 6.5 | | | | | | | | | | | | |
| Histidine | 17.8 | 20.0 | 19.9 | 26.2 | 65.3 | 67.6 | 65.7 | 53.8 | 17.0 | 12.4 | 14.4 | 20.0 |
| pH 7.0 | | | | | | | | | | | | |

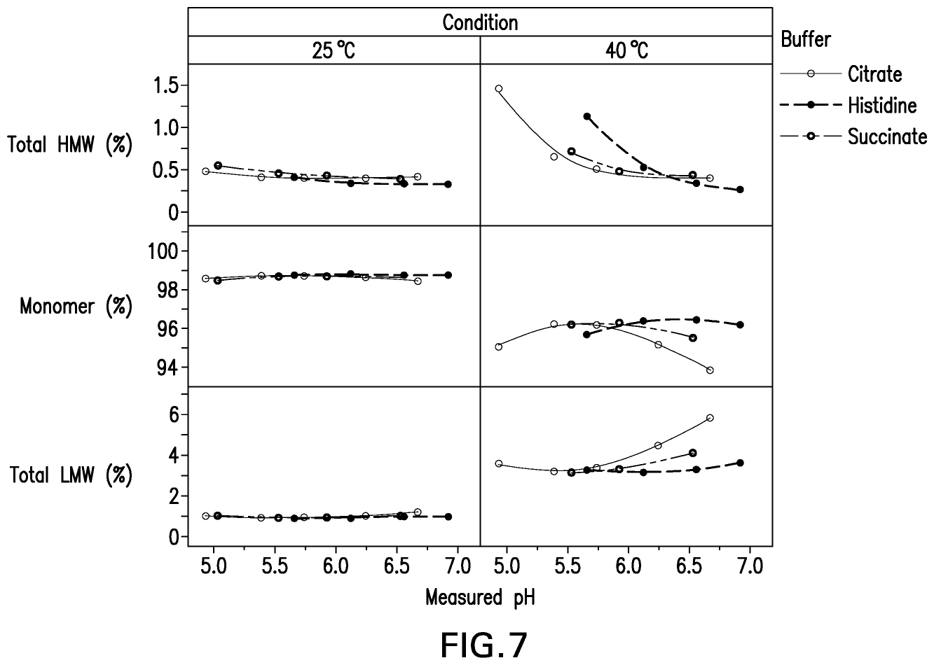

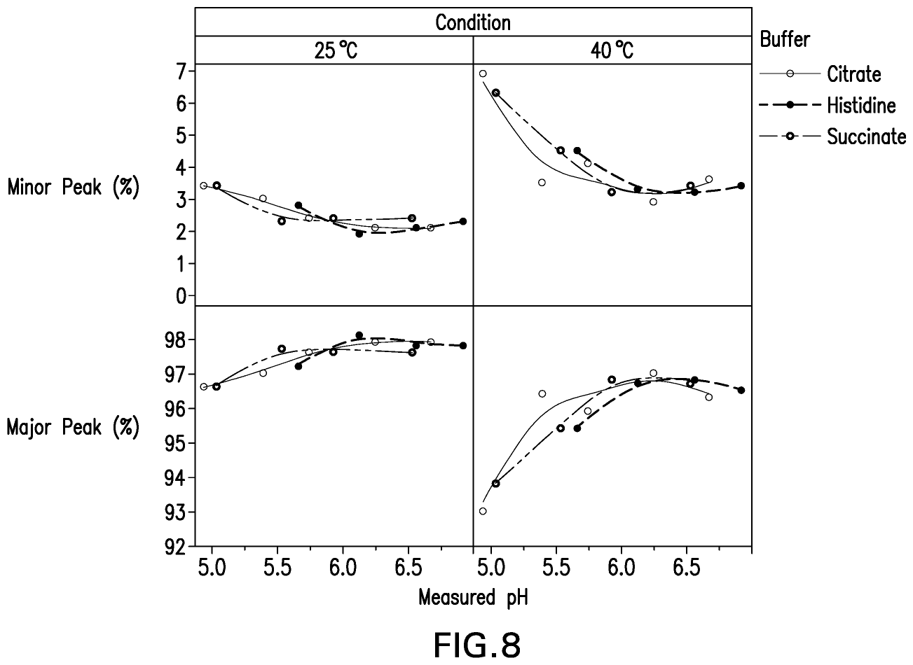

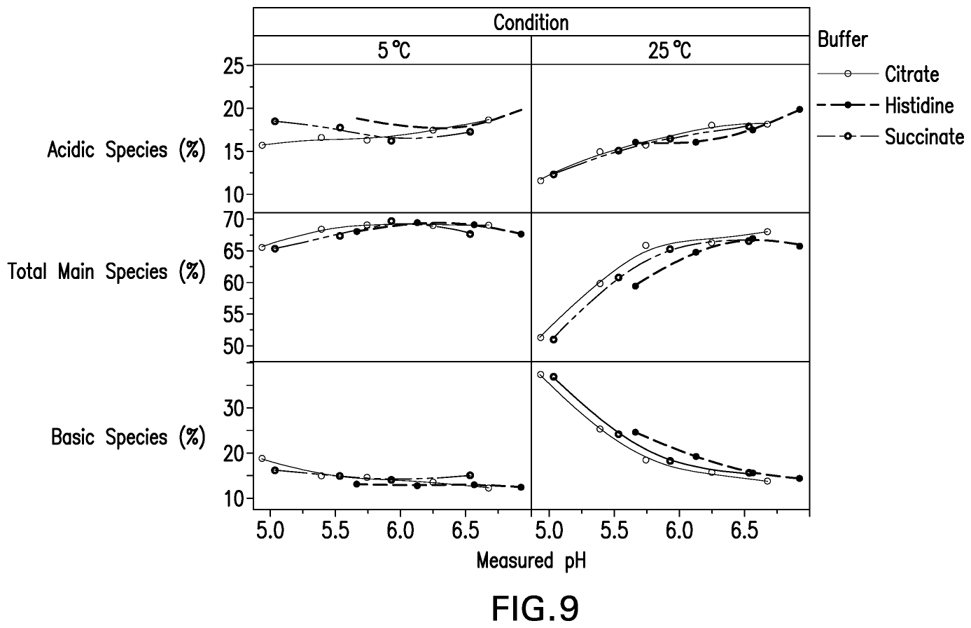

The results of this study as shown in FIG. 7 showed that citrate and succinate have comparable performance between pH 5.3 and 6.3 (measured pH) for best thermal stability with respect to HP-SEC based on 25° C. stability results. Higher amounts of fragmentation (LMW %) observed by HP-SEC at 40° C. in the citrate and succinate buffer systems, was attributed to the nature of stress and to method integration challenges. Observations made with HP-SEC were also consistent with CE-SDS non-reduced data shown in FIG. 8. Charge profile characterization by desialylated iCIEF shown in FIG. 9, also confirmed the pH range of optimum stability to be between 5.3-6.3. Formulation in histidine buffer requires a slightly higher pH range between 6.0 and 6.5 to have comparable performance to citrate and succinate; however, this buffer system also exhibited lower Tm1, indicative of lower biophysical stability.

Results from this study demonstrated that the ActRIIa-hFc fusion protein SEQ ID NO: 32 is biophysically and chemically stable in the range of 5.3-6.3 (measured pH) in both citrate and succinate buffers. Additionally, this range offered the potential to decrease the rate of possible deamidation by selecting a pH that is less basic in nature, as observed typically with biologics. So, a target pH of 5.8 was selected with citrate as the buffer system for further product development.

### Physical Stability of the ActRIIa-hFc Fusion Protein of SEQ ID NO: 32

A study was conducted to evaluate the physical stability of the ActRIIa-hFc fusion protein of SEQ ID NO: 32 under physical stress from agitation in the absence of any excipients in one of the lead buffer systems, 10 mM citrate, as a worst-case scenario. The ActRIIa-hFc fusion protein of SEQ ID NO: 32 was diluted to 5 and 50 mg/mL concentrations in 10 mM citrate, pH 5.8. The two formulations were agitated alongside non-agitated “control” samples, where 4 mL of sample was filled in 10 mL Type I glass vials with an analog shaker for up to 4 days and analyzed for loss of monomer by HP-SEC and summarized in Table 6.

TABLE 6
Physical Stability by Agitation and Impact of Monomer (%)

| Protein | | | | | | |
|---|---|---|---|---|---|---|
| Concentration | | Time Point; Monomer (%) | | | | |
| (mg/mL) | Condition | 0 | 1 hr | 4 hrs | 8 hrs | 4 days |
| 5 | agitation | 99.3 | 99.4 | 99.4 | 99.5 | 99.6 |
| 5 | control | 99.4 | 99.4 | 99.4 | 99.5 | 99.3 |
| 50 | agitation | 99.5 | 99.4 | 99.4 | 99.4 | 99.3 |
| 50 | control | 99.4 | 99.4 | 99.2 | 99.4 | 99.0 |

No significant change in monomer content was observed between the control and stressed samples for up to 4 days of agitation, irrespective of the protein concentration. The results of this study suggest that even in the absence of a bulking sugar and a surfactant, the ActRIIa-hFc fusion protein of SEQ ID NO: 32 is biophysically stable against shear and interfacial stress.

### The ActRIIa-hFc Fusion Protein of SEQ ID NO: 32 Concentration Ranging Study

A study was conducted to evaluate the short-term biophysical stability of the ActRIIa-hFc fusion protein of SEQ ID NO: 32 at various concentrations, the ActRIIa-hFc fusion protein of SEQ ID NO: 32 was prepared from 20 mg/mL to 200 mg/mL in 10 mM citrate buffer, pH 5.8 without any other excipients and filled into Type I glass vials and placed on short-term stability at 5° C. and 25° C. for up to 7 days. The purity of the formulations was measured by HP-SEC analysis over the course of the study and summarized in Table 7.

TABLE 7
HP-SEC Data for Concentration Ranging Study

| Protein | Time Point (Days, D); Monomer (%) | | | | | | | |
|---|---|---|---|---|---|---|---|---|
| Concentration | 5° C. | | | | 25° C. | | | |
| (mg/mL) | Initial | 1 D | 3 D | 7 D | Initial | 1 D | 3 D | 7 D |
| 200 | 99.6 | 99.6 | 99.4 | 99.4 | 99.5 | 99.3 | 98.8 | 98.7 |
| 150 | 99.6 | 99.6 | 99.5 | 99.5 | 99.6 | 99.5 | 99.2 | 99.1 |
| 100 | 99.6 | 99.6 | 99.6 | 99.6 | 99.6 | 99.6 | 99.4 | 99.3 |
| 75 | 99.7 | 99.6 | 99.6 | 99.6 | 99.6 | 99.6 | 99.5 | 99.5 |
| 50 | 99.6 | 99.7 | 99.6 | 99.6 | 99.6 | 99.6 | 99.6 | 99.5 |
| 20 | 99.6 | 99.7 | 99.2 | 99.7 | 99.7 | 99.7 | 99.6 | 99.6 |

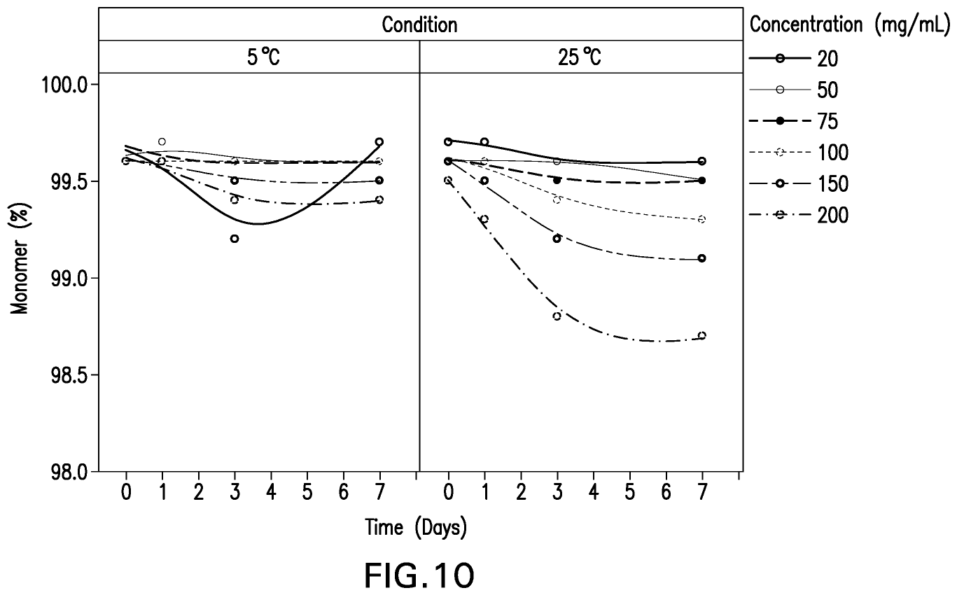

There was no significant change in purity for any of the formulations while being stored at 5° C. for up to 7 days. However, it was observed that concentrations above 75 mg/mL had higher monomer loss during short term storage at 25° C. and this trend continued with increasing concentration up to 200 mg/mL as shown in FIG. 10. The results of this study demonstrated that protein concentrations at 75 mg/mL and below had an insignificant impact on the ActRIIa-hFc fusion protein of SEQ ID NO: 32 biophysical stability. Therefore, the target concentration of the ActRIIa-hFc fusion protein of SEQ ID NO: 32 was chosen to be ≤75 mg/mL based on agitation and concentration ranging study.

### Evaluation of Long-Term Stability of Lead Buffers to Support Drug Substance Composition

A long-term stability study was conducted to compare the performance of two buffer systems at two different concentrations (based on early understanding of clinical dosing need), the ActRIIa-hFc fusion protein of SEQ ID NO: 32 was prepared at 75 and 50 mg/mL in 10 mM succinate and 10 mM citrate at pH 5.8 and filled into Type I glass vials and stored at −80° C., −20° C., 5° C., and 25° C. for up to 30 months at each storage condition.

The stability data by HP-SEC analysis is provided Table 8 and the following observations are made:

TABLE 8
HP-SEC Data for Long-Term Stability of Lead Buffers

| Protein | | Time Point (Months, M); Monomer (%) | | | | | | | | |
|---|---|---|---|---|---|---|---|---|---|---|
| Concentration | | | −80° C. | | | 5° C. | | | 25° C. | |
| (mg/mL) | Buffer System | Initial | 6 M | 12 M | 24 M | 6 M | 12 M | 24 M | 6 M | 12 M |
| 75 | 10 mM citrate, pH 5.8 | 97.9 | 98.2 | 98.1 | 98.2 | 96.0 | 95.7 | 94.6 | 93.3 | 91.2 |
| 50 | 10 mM citrate, pH 5.8 | 98.0 | 98.1 | 98.4 | 98.2 | 96.6 | 96.7 | 95.8 | 94.8 | 93.4 |
| 75 | 10 mM succinate, pH 5.8 | 97.5 | 98.3 | 97.9 | 98.6 | 95.5 | 94.4 | 93.2 | 91.8 | 90.1 |

TABLE 8
HP-SEC Data for Long-Term Stability of Lead Buffers

| Protein | | Time Point (Months, M); Monomer (%) | | | | | | | | |
|---|---|---|---|---|---|---|---|---|---|---|
| Concentration | | | −80° C. | | | 5° C. | | | 25° C. | |
| (mg/mL) | Buffer System | Initial | 6 M | 12 M | 24 M | 6 M | 12 M | 24 M | 6 M | 12 M |
| 50 | 10 mM succinate, pH 5.8 | 97.5 | 97.8 | 98.0 | 97.8 | 96.4 | 95.6 | 94.4 | 94.0 | 92.2 |

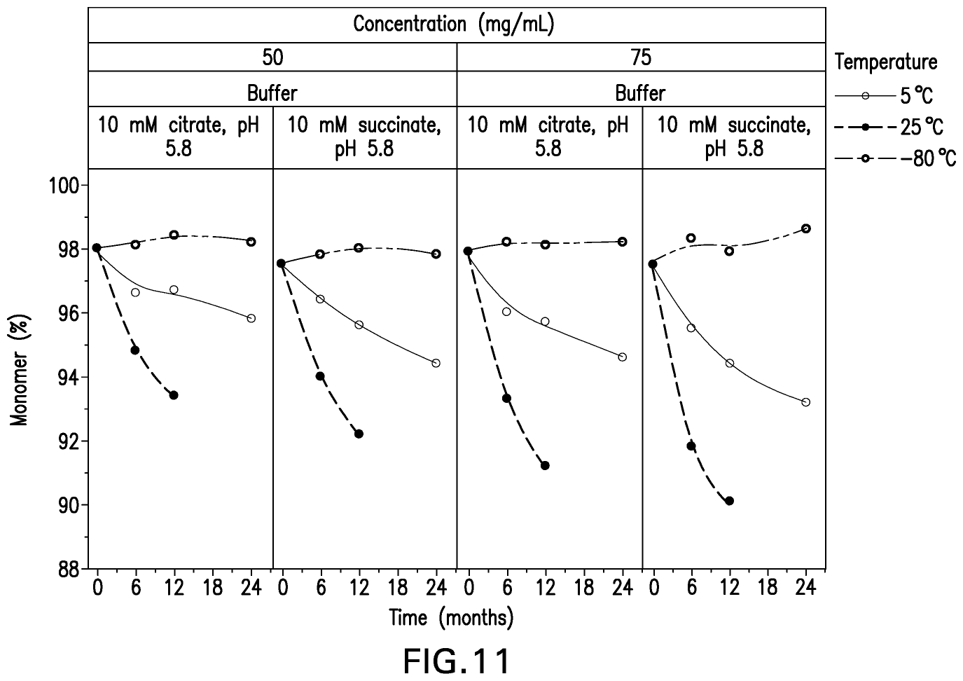

There was no significant change in purity by HP-SEC after 24 months at −80° C. with all formulations retaining ≥97.8% monomer. After 24 months at 5° C. the ActRIIa-hFc fusion protein of SEQ ID NO: 32 decreased in monomer to 94-95% for citrate formulations and 93-94% for succinate formulations. These results demonstrate that the ActRIIa-hFc fusion protein of SEQ ID NO: 32 is biophysically stable as a solution even in the absence of any surfactant or a stabilizing sugar, since none of the formulations had a % monomer loss below 94% monomer even after 2 years at 2-8° C. After 12 months at 25° C. the ActRIIa-hFc fusion protein of SEQ ID NO: 32 decreased in monomer to 91-93% for citrate formulations and 90-92% for succinate formulations. In both 5° C. and 25° C. storage data the 75 mg/mL drug substance degraded slightly more than when at 50 mg/mL for each buffer system as expected and these trends are shown in FIG. 11.

In conclusion, the data from this study demonstrates that the ActRIIa-hFc fusion protein of SEQ ID NO: 32 in the citrate buffer has slightly better long-term stability than succinate buffer at 5° C. and 25° C. storage but is identical at −80° C., which is the recommended storage condition. Therefore, 10 mM citrate at pH 5.8 was chosen as the target formulation for the SEQ ID NO: 32 DS under frozen conditions.

### Salt Concentration Ranging Study

The ActRIIa-hFc fusion protein of SEQ ID NO: 32 was prepared in a range of sodium chloride concentrations from 0 to 150 mM in 10 mM citrate pH 5.8 at 50 mg/mL the ActRIIa-hFc fusion protein of SEQ ID NO: 32 with 8% (weight/volume) sucrose to evaluate the effect of buffer strength on its biophysical properties. These formulations were evaluated for their thermal stability using DSC and their diffusion interaction parameter (kD) by DLS. The results are summarized in Table 9.

TABLE 9
Salt Concentration Ranging Study: Effect of Sodium Chloride
on the Melting Point and Diffusion Interaction Parameter (kD)

| Sodium | DSC | DLS |
|---|---|---|
| Chloride (mM) | Tm1 (° C.) | k_{D} (mL/g) |
| 0 | 69.8 | 16.2 |
| 30 | 70.1 | 9.6 |
| 50 | 70.0 | 5.8 |
| 70 | 70.0 | 3.5 |
| 100 | 69.8 | 5.3 |
| 150 | 69.4 | 4.5 |

The results of the study showed that the highest kD of the ActRIIa-hFc fusion protein of SEQ ID NO: 32 was without sodium chloride present (16.2 mL/g) and would decrease with higher concentrations down to around 5 mL/g plateauing at concentrations above 50 mM. The more positive a molecules kD value indicates stronger repulsion between molecules, which is indicative of better colloidal stability suggesting that the ActRIIa-hFc fusion protein of SEQ ID NO: 32 has the best colloidal stability without any sodium chloride. Additionally, the melting points of the ActRIIa-hFc fusion protein of SEQ ID NO: 32 were largely identical and sufficiently high for all concentrations of sodium chloride. Based on this data, sodium chloride was not deemed to be suitable for further development work in the stabilization of the ActRIIa-hFc fusion protein of SEQ ID NO: 32.

### Excipient Screening Study

A screening study was designed to evaluate a range of excipients for the purpose of increasing the biophysical stability of the ActRIIa-hFc fusion protein of SEQ ID NO: 32 in solution. In this excipient screen, the ActRIIa-hFc fusion protein of SEQ ID NO: 32 was formulated to a concentration of 2 mg/mL with a variety of excipients: a salt (sodium chloride), sugars (sucrose, mannitol) and amino acids (arginine, histidine). These solutions as summarized in Table 10 were placed on 5° C., 25° C., and 45° C. stability for up to 12 months, 9 months, and 3 months respectively and subsequently tested for their purity (% monomer content) by HP-SEC.

TABLE 10
Excipient Screening Study: Effect of Excipients
on % Monomer Species by HP-SEC

| | Excipient | | 12 months | 9 months | 3 months |
|---|---|---|---|---|---|
| Excipient | Concentration | Initial | 5° C. | 25° C. | 45° C. |
| Arginine | 0.1% | 99.7 | 99.8 | 99.5 | 97.8 |
| | (weight/volume) | | | | |
| | 0.5% | 99.7 | 99.8 | 99.6 | 97.7 |
| | (weight/volume) | | | | |
| | 2.0% | 99.7 | 99.8 | 99.0 | 97.7 |
| | (weight/volume) | | | | |
| Histidine | 0.1% | 99.8 | 99.8 | 99.2 | 96.8 |
| | (weight/volume) | | | | |
| | 0.5% | 99.7 | 99.8 | 98.2 | 94.7 |
| | (weight/volume) | | | | |
| | 2.0% | 99.7 | 99.9 | 96.7 | 90.5 |
| | (weight/volume) | | | | |
| Mannitol | 0.1% | 99.7 | 99.8 | 99.5 | 98.8 |
| | (weight/volume) | | | | |
| | 0.5% | 99.7 | 99.8 | 99.2 | 97.9 |
| | (weight/volume) | | | | |
| | 2.0% | 99.7 | 99.8 | 99.3 | 98.0 |
| | (weight/volume) | | | | |
| Sucrose | 0.1% | 99.8 | 99.8 | 99.5 | 97.8 |
| | (weight/volume) | | | | |
| | 0.5% | 99.8 | 99.8 | 99.3 | 97.9 |
| | (weight/volume) | | | | |
| | 2.0% | 99.8 | 99.8 | 99.5 | 98.0 |
| | (weight/volume) | | | | |

All formulations were biophysically stable at 5° C. and 25° C. storage temperatures with >99% monomer retained after both 12 months at 5° C. and 9 months at 25° C. The evaluated excipients demonstrated comparable biophysical stability at the stressed condition (45° C.) after 3 months retaining 97-99% monomer, except for histidine. Increasing the concentration of histidine was correlated with a decrease in biophysical stability with up to 2% (weight/volume) histidine resulting in 6% less monomer than the 0.1% (weight/volume) histidine formulation.

In conclusion, the ActRIIa-hFc fusion protein of SEQ ID NO: 32 was identified to be biophysically stable in all prototypes tested.

### Surfactant Screening Study

Surfactants are widely used to stabilize proteins from shear and interfacial stresses encountered during manufacturing, shipping and handling. Therefore, a series of experiments were conducted to evaluate polysorbate 80 (PS80) as a surfactant to stabilize SEQ ID NO: 32 under various stress conditions.

### Study 1: Surfactant Screening by Thermal Stability

A PS80 concentration ranging stability study was conducted to evaluate the effect of PS80 on the long-term stability of the ActRIIa-hFc fusion protein of SEQ ID NO: 32. In this study the ActRIIa-hFc fusion protein of SEQ ID NO: 32 was formulated to a concentration of 50 mg/mL with varying levels of PS80 ranging from 0.001% to 0.05% in 10 mM citrate at pH 5.8 with 8% (weight/volume) sucrose. These formulations were filled into Type I glass vials and placed on stability at 5° C. and 25° C. for up to 24 months and 45° C. for up to 3 months. The purity of the formulations was measured by HP-SEC over the course of the study to monitor their physical stability. The stability data by HP-SEC is provided in Table 11.

TABLE 11
PS80 Concentration Ranging: Effect of PS80 Concentration
on % Monomer Species at Varying Storage Conditions

| | Time Point (Months, M); Monomer (%) | | | | | | | |
|---|---|---|---|---|---|---|---|---|
| PS80 | | 5° C. | | | 25° C. | | 45° C. | |
| (%) | Initial | 6 M | 12 M | 24 M | 12 M | 24 M | 1 M | 3 M |
| 0.001 | 99.5 | 99.3 | 99.3 | 99.2 | 98.1 | 96.6 | 97.7 | 93.5 |
| 0.020 | 99.5 | 99.3 | 99.3 | 99.2 | 98.2 | 96.5 | 97.8 | 93.5 |
| 0.050 | 99.5 | 99.4 | 99.3 | 99.2 | 98.1 | 96.6 | 97.8 | 93.6 |

No significant differences in biophysical stability profiles were observed between the varying levels of PS80, where all formulations had >99% monomer after 24 months at 5° C., >96% monomer after 24 months at 25° C., and >93% monomer after 3 months 45° C. The results of this study demonstrated that PS80 concentration between 0.001% and 0.05% were comparable with regards to biophysical stability under thermal stress.

### Study 2: Surfactant Screening by Agitation and Freeze Thaw

An additional PS80 concentration ranging study was conducted to further evaluate the effect of PS80 in stabilizing the ActRIIa-hFc fusion protein of SEQ ID NO: 32 against agitation as well as freeze thaw stress. In this study the ActRIIa-hFc fusion protein of SEQ ID NO: 32 was formulated to a concentration of 50 mg/mL with varying levels of PS80 ranging from 0% to 0.05% in 10 mM citrate at pH 5.8 with 8% (weight/volume) sucrose. These formulations were filled into Type I glass vials and agitated at 300 RPM for up to 7 days. Separately, the formulations were also stressed with up to 8 freeze thaw cycles from −80° C. to 25° C. The purity of the formulations was measured by UP-SEC, particulates by HIAC and MFI, and charge variants by desialylated iCIEF over the course of the study to monitor the biophysical and chemical stability of SEQ ID NO: 32. The stability data is provided in Table 12 through Table 19.

TABLE 12
Surfactant Screening by Agitation: Effect of PS80 Concentration
on the HMW, Monomer, LMW by UP-SEC and Major Peak by CE-SDS NR

| | UP-SEC | | | | | | | | | CE-SDS NR | | |
|---|---|---|---|---|---|---|---|---|---|---|---|---|
| | Time Point (Days, D); | | | Time Point (Days, D); | | | Time Point (Days, D); | | | Time Point (Days, D); | | |
| PS80 | HMW (%) | | | Monomer (%) | | | LMW (%) | | | Major Peak (%) | | |
| (%) | Control | 3 D | 7 D | Control | 3 D | 7 D | Control | 3 D | 7 D | Control | 3 D | 7 D |
| 0.000 | 0.8 | 0.8 | 0.8 | 97.3 | 97.3 | 97.3 | 1.9 | 1.9 | 1.9 | 98.1 | 98.0 | 98.2 |
| 0.005 | 0.8 | 0.8 | 0.8 | 97.4 | 97.2 | 97.3 | 1.9 | 2.0 | 1.9 | 98.0 | 98.2 | 98.4 |
| 0.010 | 0.8 | 0.8 | 0.8 | 97.3 | 97.3 | 97.3 | 1.9 | 1.9 | 1.9 | 98.1 | 98.2 | 97.9 |
| 0.020 | 0.8 | 0.8 | 0.8 | 97.3 | 97.3 | 97.3 | 1.9 | 1.9 | 1.9 | 98.0 | 98.0 | 98.0 |
| 0.030 | 0.8 | 0.8 | 0.8 | 97.3 | 97.3 | 97.3 | 1.9 | 2.0 | 2.0 | 97.8 | 98.2 | 98.0 |
| 0.050 | 0.8 | 0.8 | 0.8 | 97.2 | 97.3 | 97.2 | 2.0 | 1.9 | 2.0 | 97.9 | 98.1 | 97.9 |

TABLE 13
Surfactant Screening by Agitation: Effect of PS80 Concentration on Particulate Matter by MFI

| | Time Point (Days, D); ≥2 | | | Time Point (Days, D); ≥5 | | | Time Point (Days, D); ≥10 | | | Time Point (Days, D); ≥25 | | |
|---|---|---|---|---|---|---|---|---|---|---|---|---|
| PS80 | μm Particulates per mL | | | μm Particulates per mL | | | μm Particulates per mL | | | μm Particulates per mL | | |
| (%) | Control | 3 D | 7 D | Control | 3 D | 7 D | Control | 3 D | 7 D | Control | 3 D | 7 D |
| 0.000 | 173 | 157 | 175 | 40 | 28 | 44 | 14 | 12 | 12 | 0 | 0 | 0 |
| 0.005 | 35 | 35 | 51 | 5 | 7 | 5 | 0 | 5 | 5 | 0 | 0 | 0 |
| 0.010 | 115 | 53 | 88 | 10 | 5 | 12 | 0 | 3 | 3 | 0 | 0 | 3 |
| 0.020 | 39 | 72 | 129 | 7 | 12 | 23 | 3 | 3 | 7 | 0 | 0 | 3 |
| 0.030 | 115 | 65 | 69 | 19 | 23 | 14 | 3 | 12 | 3 | 0 | 0 | 0 |
| 0.050 | 125 | 161 | 129 | 23 | 37 | 37 | 10 | 12 | 7 | 0 | 3 | 0 |

TABLE 14
Surfactant Screening by Agitation: Effect of PS80 Concentration on Particulate Matter by HIAC

| | Time Point (Days, D); ≥2 | | | Time Point (Days, D); ≥5 | | | Time Point (Days, D); ≥10 | | | Time Point (Days, D); ≥25 | | |
|---|---|---|---|---|---|---|---|---|---|---|---|---|
| PS80 | μm Particulates per mL | | | μm Particulates per mL | | | μm Particulates per mL | | | μm Particulates per mL | | |
| (%) | Control | 3 D | 7 D | Control | 3 D | 7 D | Control | 3 D | 7 D | Control | 3 D | 7 D |
| 0.000 | 77 | 50 | 39 | 15 | 14 | 7 | 5 | 4 | 1 | 1 | 1 | 0 |
| 0.020 | 16 | 34 | 35 | 7 | 8 | 6 | 2 | 2 | 2 | 1 | 0 | 0 |

TABLE 15
Surfactant Screening by Agitation: Effect of PS80
Concentration on Acidic Species, Basic Species,
and Total Main Species by Desialylated iCIEF

| | Timepoint (Days, | | | Timepoint (Days, | | | Timepoint (Days, | | |
|---|---|---|---|---|---|---|---|---|---|
| | D); Acidic | | | D); Total Main | | | D); Basic | | |
| | Species (%) | | | Species (%) | | | Species (%) | | |
| PS80 | Con- | 3 | 7 | Con- | 3 | 7 | Con- | 3 | 7 |
| (%) | trol | D | D | trol | D | D | trol | D | D |
| 0.000 | 22.7 | 20.8 | 21.1 | 67.9 | 69.3 | 69.1 | 9.4 | 10.0 | 9.8 |
| 0.005 | 20.7 | 20.8 | 22.1 | 68.3 | 69.1 | 68.4 | 11.1 | 10.2 | 9.6 |
| 0.010 | 21.8 | 22.6 | 22.7 | 68.5 | 67.7 | 68.5 | 9.8 | 9.8 | 8.9 |
| 0.020 | 20.7 | 21.5 | 22.6 | 69.3 | 69.1 | 67.8 | 10.1 | 9.5 | 9.7 |
| 0.030 | 20.9 | 22.7 | 23.4 | 69.3 | 68.2 | 68.9 | 9.9 | 9.1 | 7.8 |
| 0.050 | 20.9 | 21.9 | 21.0 | 69.4 | 68.6 | 69.3 | 9.7 | 9.6 | 9.9 |

TABLE 16
Surfactant Screening by Freeze-Thaw: Effect of PS80 Concentration
on the HMW, Monomer, LMW by UP-SEC data, Major Peak by CE-SDS
Non-Reduced of SEQ ID NO: 32 Upon Freeze Thaw Stress

| | UP-SEC | | | | | | | | | CE-SDS NR | | |
|---|---|---|---|---|---|---|---|---|---|---|---|---|
| | Freeze Thaw Cycles; | | | Freeze Thaw Cycles; | | | Freeze Thaw Cycles; | | | Freeze Thaw Cycles; | | |
| PS80 | HMW (%) | | | Monomer (%) | | | LMW (%) | | | Major Peak (%) | | |
| (%) | 0 | 3 | 5 | 0 | 3 | 5 | 0 | 3 | 5 | 0 | 3 | 5 |
| 0.000 | 0.8 | 0.8 | 0.8 | 97.3 | 97.4 | 97.4 | 1.9 | 1.8 | 1.9 | 98.0 | 98.1 | 98.1 |
| 0.010 | 0.8 | 0.8 | 0.8 | 97.4 | 97.4 | 97.4 | 1.9 | 1.8 | 1.8 | 98.0 | 98.1 | 98.1 |
| 0.020 | 0.8 | 0.8 | 0.8 | 97.4 | 97.3 | 97.3 | 1.9 | 1.9 | 1.9 | 98.0 | 98.2 | 98.1 |
| 0.030 | 0.8 | 0.8 | 0.8 | 97.3 | 97.3 | 97.3 | 2.0 | 1.9 | 1.9 | 98.1 | 98.2 | 98.0 |

TABLE 17
Surfactant Screening by Freeze-Thaw: Effect of PS80
Concentration on the Charge Variants by Desialylated
iCIEF of SEQ ID NO: 32 Upon Freeze Thaw Stress

| | Freeze Thaw | | | Freeze Thaw | | | Freeze Thaw | | |
|---|---|---|---|---|---|---|---|---|---|
| | Cycles; Acidic | | | Cycles; Total | | | Cycles; Basic | | |
| PS80 | Species (%) | | | Main Species (%) | | | Species (%) | | |
| (%) | 0 | 3 | 5 | 0 | 3 | 5 | 0 | 3 | 5 |
| 0.000 | 21.4 | 22.0 | 20.4 | 68.7 | 68.4 | 69.2 | 9.9 | 9.7 | 10.5 |
| 0.010 | 21.9 | 23.0 | 20.7 | 69.2 | 67.5 | 69.6 | 9.0 | 9.5 | 9.8 |
| 0.020 | 22.4 | 20.8 | 20.1 | 68.2 | 69.3 | 70.5 | 9.5 | 9.9 | 9.6 |
| 0.030 | 22.2 | 20.5 | 20.0 | 68.6 | 69.5 | 70.3 | 9.4 | 10.1 | 9.8 |

TABLE 18
Surfactant Screening by Freeze-Thaw: Effect of PS80 Concentration on
Particulate Matter by HIAC of SEQ ID NO: 32 upon Freeze Thaw Stress

| | Freeze Thaw Cycles; ≥2 | | | Freeze Thaw Cycles; ≥5 | | | Freeze Thaw Cycles; ≥10 | | | Freeze Thaw Cycles; ≥25 | | |
|---|---|---|---|---|---|---|---|---|---|---|---|---|
| PS80 | μm Particulates per mL | | | μm Particulates per mL | | | μm Particulates per mL | | | μm Particulates per mL | | |
| (%) | 0 | 3 | 5 | 0 | 3 | 5 | 0 | 3 | 5 | 0 | 3 | 5 |
| 0.000 | 60 | 81 | 88 | 15 | 19 | 14 | 4 | 6 | 2 | 1 | 1 | 1 |
| 0.020 | 27 | 65 | 82 | 6 | 9 | 12 | 1 | 2 | 6 | 0 | 1 | 1 |

TABLE 19
Surfactant Screening by Freeze-Thaw: Effect of PS80 Concentration on
Particulate Matter by MFI of SEQ ID NO: 32 upon Freeze Thaw Stress

| | Freeze Thaw Cycles; ≥2 | | | Freeze Thaw Cycles; ≥5 | | | Freeze Thaw Cycles; ≥10 | | | Freeze Thaw Cycles; ≥25 | | |
|---|---|---|---|---|---|---|---|---|---|---|---|---|
| PS80 | μm Particulates per mL | | | μm Particulates per mL | | | μm Particulates per mL | | | μm Particulates per mL | | |
| (%) | 0 | 3 | 5 | 0 | 3 | 5 | 0 | 3 | 5 | 0 | 3 | 5 |
| 0.000 | 134 | 230 | 343 | 37 | 46 | 104 | 5 | 7 | 28 | 0 | 3 | 5 |
| 0.010 | 33 | 74 | 113 | 5 | 14 | 7 | 0 | 0 | 0 | 0 | 0 | 0 |
| 0.020 | 56 | 147 | 115 | 14 | 26 | 30 | 7 | 0 | 5 | 5 | 0 | 3 |
| 0.030 | 44 | 145 | 129 | 21 | 35 | 44 | 5 | 12 | 12 | 0 | 3 | 0 |

There was no significant difference in the purity of any of the tested formulations for HMW, monomer, or LMW content by UP-SEC when compared to control sample. There was no discernible trend across the tested formulation prototypes by CE-SDS or charge variants by desialylated iCIEF when compared to the control. A slight reduction in 2 μm and 5 μm particulates were observed starting with at least 0.01% PS80 from freeze thaw stress as compared to 0% PS80.

The results of this study demonstrated that there is no significant difference seen between samples containing 0.01-0.05% PS-80 after exposure to agitation or freeze/thaw stress. These studies did not discriminate between the various PS80 concentrations; however, a 0.02% (weight/volume) of PS80 was selected to ensure product quality and minimizing stability risk while maintaining safety in support of emerging supply chain.

### Sucrose Concentration Ranging Study

To ensure that an injection solution of the ActRIIa-hFc fusion protein of SEQ ID NO: 32 had a tonicity amenable for parenteral administration (260-380 mOsm/kg) a range of sucrose concentrations outlined in Table 20 were prepared in 10 mM citrate at pH 5.8 and osmolality measured. Based on these preparations, 8-10% (weight/volume) was determined to be appropriate for formulation optimization studies described below.

TABLE 20
Osmolality with Varying Sucrose in Platform Composition

| Sucrose (%) (weight/volume) | Osmolality (mOsm/kg) |
|---|---|
| 2 | 103 |
| 4 | 169 |
| 6 | 239 |
| 8 | 320 |
| 10 | 388 |
| 12 | 489 |
| 14 | 560 |

Formulations comprising 50 mg/mL of the ActRIIa-hFc fusion protein of SEQ ID NO: 32, 8% to 10% (weight/volume) sucrose in 10 mM citrate at pH 5.8 with 0.02% (weight/volume) PS80 were prepared and evaluated for purity by HP-SEC to monitor their physical stability under different conditions. For the thermal stability arm of the study, formulations were placed on stability at 5° C. and 25° C. for up to 24 months and 45° C. for up to 3 months. The stability data by HP-SEC analysis is provided in Table 21.

TABLE 21
Sucrose Concentration Ranging Study: Effect of Sucrose
Concentration on % Monomer by HP-SEC on Stability

| | Time Point (Months, M); Monomer (%) | | | | | | |
|---|---|---|---|---|---|---|---|
| Sucrose | | 5° C. | | 25° C. | | 45° C. | |
| (%) | Initial | 12 M | 24 M | 6 M | 12 M | 1 M | 3 M |
| 8 | 99.4 | 99.3 | 99.1 | 99.1 | 98.1 | 98.2 | 92.3 |
| 9 | 99.4 | 99.3 | 99.0 | 98.7 | 98.1 | 98.3 | 92.4 |
| 10 | 99.4 | 99.3 | 99.1 | 98.6 | 98.1 | 98.4 | 92.5 |

For the agitation arm of the study the formulations were filled into 3 mL Type I glass vials and shaken at 250 RPM on a plate shaker for up to 7 days. The agitation data by HP-SEC analysis is provided in Table 22.

TABLE 22
Sucrose Concentration Ranging Study: Effect of Sucrose
Concentration on % Monomer by HP-SEC on Agitation

| | |
|---|---|
| Sucrose | Time Point (Days, D); Monomer (%) |

| (%) | Initial | 1 D | 3 D | 7 D |
|---|---|---|---|---|
| 8 | 98.6 | 98.7 | 98.6 | 98.8 |
| 9 | 98.5 | 98.7 | 98.7 | 98.7 |
| 10 | 98.6 | 98.6 | 98.7 | 98.8 |

For the photostability arm of the study another set of vials were filled with 1 mL of each formulation and split into two sets for incubation in a photostability chamber: one exposed to visible light at 25 kLux for 48 hours (1.2 mLuxhr) and another exposed to 20 W/m2 of UV light for 10 hours (200 Wh/m2). Two control vials were also prepared for each formulation: one protected from light by aluminum foil in the photostability chamber and another kept at 5° C. in a refrigerator. The photostability data by HP-SEC analysis is provided in Table 23.

TABLE 23
Sucrose Concentration Ranging Study: Effect of Sucrose
Concentration on % Monomer by HP-SEC on Photostability

| | Monomer (%) | | | | | | |
|---|---|---|---|---|---|---|---|
| | Visible Light (1.2 mLuxhr) | | | UV Light (200 Wh/m²) | | | |
| Sucrose | Dark | Light | Differ- | Dark | Light | Differ- | 5° C. |
| (%) | Control | Exposed | ence | Control | Exposed | ence | Control |
| 8 | 99.4 | 83.8 | −15.6 | 99.5 | 90.1 | −9.4 | 99.4 |
| 9 | 99.5 | 82.7 | −16.8 | 99.4 | 90.3 | −9.1 | 99.4 |
| 10 | 99.4 | 83.3 | −16.1 | 99.3 | 90.5 | −8.8 | 99.4 |

There were no significant differences in stability profiles between the varying levels of sucrose with each formulation having 99% monomer after 24 months at 5° C., 98% monomer after 24 months at 25° C., and 92% monomer after 3 months 45° C. There was no discernible trend in purity for any of the formulations after 7 days of agitation with all formulations retaining >98% monomer. For the photostability arm of the study, each formulation decreased in % monomer by approximately 16-17% after visible light exposure and approximately 9-10% after UV light exposure, highlighting a potential degradation pathway.

In conclusion, sucrose concentrations between 8% to 10% (weight/volume) had no significant impact on the long-term stability profile of the ActRIIa-hFc fusion protein of SEQ ID NO: 32, when protected from light, or under physical stress due to aggregation. It was also observed that the ActRIIa-hFc fusion protein of SEQ ID NO: 32 is prone to light instability, a complex mechanism to delineate. Hence, a lyophilized formulation which decreases the potential for chemical instability due to light was developed. An 8% (weight/volume) concentration of sucrose was deemed sufficient for lyophilized formulation development.

### Protein Concentration Ranging Study

A protein concentration ranging stability study was conducted to evaluate the effect of increasing the protein concentration on the long-term stability profile. In this study, the ActRIIa-hFc fusion protein of SEQ ID NO: 32 was formulated at concentrations varying from 50 to 100 mg/mL in 10 mM citrate, 8% (weight/volume) sucrose, 0.02% (weight/volume) PS80. These formulations were filled into Type I glass vials and placed on stability for 24 months at 5° C. and 3 months at 45° C. The purity of the formulations was measured by HP-SEC analysis over the course of the study to monitor their physical stability and summarized in Table 24.

TABLE 24
SEC Data Protein Concentration Ranging Study

| Protein | Time Point (Months, M); Monomer (%) | | | | | | |
|---|---|---|---|---|---|---|---|
| Concentration | | 5° C. | | | 45° C. | | |
| (mg/mL) | Initial | 6 M | 12 M | 24 M | 1 M | 2 M | 3 M |
| 50 | 99.6 | 99.4 | 99.3 | 99.0 | 97.0 | 94.5 | 91.4 |
| 75 | 99.5 | 99.3 | 99.1 | 99.1 | 96.3 | 93.4 | 91.1 |
| 100 | 99.5 | 99.1 | 99.0 | 98.6 | 96.0 | 91.5 | 87.7 |

There was no significant difference in the 5° C. stability profiles between the different protein concentrations, where all formulations had 99% monomer after 24 months at 5° C. The 100 mg/mL concentration, however, showed slightly more degradation than the lower concentrations having 88% monomer after 3 months at stressed conditions (45° C.) while the lower concentrations retained 91% monomer.

The results of this study demonstrated that SEQ ID NO: 32 can be concentrated up to 75 mg/mL without any significant impact on the long-term stability and this early physical stability data suggested that a concentration as high as 100 mg/mL may also have sufficient long-term stability based on the 5° C. data.

### Formulation Composition

Based on the formulation development work summarized above and the potential for charge instability during long term storage at recommended storage conditions as a ready to use solution, a lyophilized formulation was developed with the optimized composition. The target pre and post lyophilization composition of the ActRIIa-hFc fusion protein of SEQ ID NO: 32 is outlined below.

TABLE 25
Proposed Formulation

| Formulation Component | Solution for Lyophilization |
|---|---|
| Protein Concentration | 50 mg/mL |
| Buffer System and pH | 10 mM sodium citrate, pH 5.8 |
| Stabilizer/Bulking Agent | 8% (weight/volume) sucrose |
| Surfactant | 0.02% (weight/volume) polysorbate 80 |

The composition was further provided in 2 vial strengths, and when reconstituted with the defined quantity of Sterile Water for Injection (SWFI), each composition contains 50 mg/mL of the ActRIIa ligand trap (active pharmaceutical ingredient), and the following excipients: 10 mM citrate, 8% (weight/volume) sucrose, and 0.02% (weight/volume) polysorbate 80 at pH 5.8.

The components present in the lyophilized ActRIIa-hFc fusion protein composition are as follows:

| | | Lyophilzed Cake | | |
|---|---|---|---|---|
| | | (mg/vial) | | |
| | | 45 | 60 | Percent by |
| Component | Function | mg/vial | mg/vial | weight |
| ActRIIa-hFc fusion | Active | 55 | 72.5 | 37.6% |
| protein | ingredient | | | |
| Citric Acid monohydrate | Buffer | 0.48 | 0.64 | 0.32% |
| Tri-sodium citrate | Buffer | 2.56 | 3.37 | 1.75% |
| dihydrate | | | | |
| Polysorbate 80 | Surfactant | 0.22 | 0.29 | 0.15% |
| Sucrose | Tonicity | 88 | 116 | 60.2% |
| | adjustment | | | |
| Total | | 146.26 mg | 192.8 mg | |

For administration, the lyophilate in the 45 mg vial is reconstituted with 1.0 mL Sterile Water For Injection (sWFI), and the lyophilate in the 60 mg vial is reconstituted with 1.3 mL SWFI. Both result in a ActRIIa ligand trap solution of at least 50 mg/mL.

### pH Robustness of Lyophilized Formulation

Studies were conducted to evaluate the robustness of critical components in the formulation. This initial study was conducted to evaluate the effect of pH on the stability of the lyophilized formulations. Formulations ranging in pH from 5.3 to 6.3 were prepared in the formulation 10 mM citrate buffer at pH 5.8 with 8% (weight/volume) sucrose, and 0.02% (weight/volume) polysorbate 80 at 50 mg/mL of protein. These formulations (2 mL) were filled into 5 mL Type I glass vials and lyophilized using an early phase lyophilization cycle and placed on stability at 25° C., 45° C., and 60° C. conditions. The purity of the formulations was measured by HP-SEC over the course of the study to monitor their physical stability and summarized in Table 26.

TABLE 26
SEC Data for pH Robustness of Product Formulation

| | Time Point (Months, M); Monomer (%) | | | | | | | |
|---|---|---|---|---|---|---|---|---|
| | | | 25° C. | | | 45° C. | | |
| pH | Pre-Lyo | Initial | 3 | 6 | 9 | 1 | 3 | 6 |
| 5.3 | 99.6 | 99.6 | 99.4 | 99.3 | 99.3 | 98.9 | 98.8 | 98.1 |
| 5.6 | 99.6 | 99.6 | 99.5 | 99.3 | 99.2 | 98.5 | 99.0 | 98.1 |
| 5.8 | 99.6 | 99.6 | 99.5 | 99.3 | 99.3 | 98.9 | 99.0 | 98.2 |
| 6.0 | 99.6 | 99.6 | 99.6 | 99.5 | 99.3 | 99.2 | 99.0 | 98.4 |
| 6.3 | 99.6 | 99.5 | 99.5 | 99.2 | 99.3 | 99.1 | 99.0 | 98.3 |

There was no significant effect of pH on the stability profile of SEQ ID NO: 32 for either 25° C. or 45° C. temperatures.

The results of this study demonstrated no significant effect on the biophysical stability of the ActRIIa-hFc fusion protein of SEQ ID NO: 32 within a pH range of 5.3-6.3 of the formulation compositions under lyophilized conditions. Also, earlier development studies showed that variation in PS 80 did not have any impact on biophysical stability in a solution form. Hence, pH and PS80 were eliminated as parameters in the formulation robustness studies under lyophilized conditions.

### Formulation Robustness Study

Formulation robustness study was conducted to determine the robustness of the formulation by varying the protein and sucrose concentration from their target levels. These two components were selected for evaluation, as they were shown to have the most significant effect in determining stability of the ActRIIa-hFc fusion protein of SEQ ID NO: 32 as a lyophilized product. Furthermore, this laboratory scale study leveraged the primary packaging and lyophilization cycle parameters for manufacturing.

Formulations ranging in protein concentration from 45 (Lo) to 55 mg/mL (Hi) and sucrose concentration from 7 (Lo) to 9% (Hi) (weight/volume) were prepared in 10 mM citrate buffer with 0.02% (weight/volume) polysorbate 80 as outlined in Table 27. 60 mg/Vial, containing 1.45 mL of each formulation was filled into 2R Type I glass vials, and lyophilized using the targeted commercial lyophilization cycle.

TABLE 27
Summary of Formulations for Formulation Robustness Study

| Level | Protein Concentration (mg/mL) | Sucrose (%) |
|---|---|---|
| Lo-Lo | 45 | 7 |
| Lo-Hi | 45 | 9 |
| Hi-Lo | 55 | 7 |
| Hi-Hi | 55 | 9 |
| Target | 50 | 8 |

All formulation prototypes, as lyophilized cakes, were placed on 5° C., 25° C., and 40° C. stability conditions for up to 18 months, 6 months, and 3 months, respectively. The prototypes were analyzed for purity by HP-SEC, fragmentation by CE-SDS NR, charge variants by desialylated iCIEF, particulates by HIAC, moisture content by Karl-Fischer pH, and reconstitution time and summarized in Table 28 through Table 38.

TABLE 28
Formulation Robustness: Reconstitution Time

| | Time Point (Months, M): Reconstitution | | | | | | | |
|---|---|---|---|---|---|---|---|---|
| | Time (minutes:seconds) | | | | | | | |
| | | 5° C. | | 25° C. | | | 40° C. | |
| Level | Initial | 6 M | 18 M | 1 M | 3 M | 6 M | 1 M | 3 M |
| Lo-Lo | 2:09 | 1:44 | 2:07 | 1:46 | 1:58 | 1:54 | 1:29 | 2:01 |
| Lo-Hi | 2:25 | 1:48 | 2:14 | 2:03 | 2:21 | 1:53 | 1:41 | 2:00 |
| Hi-Lo | 2:23 | 1:54 | 2:09 | 2:13 | 2:28 | 2:09 | 1:58 | 2:29 |
| Hi-Hi | 2:40 | 1:46 | 2:23 | 2:05 | 2:32 | 2:09 | 2:03 | 2:37 |
| Target | 1:53 | 1:38 | 2:16 | 1:44 | 1:53 | 1:43 | 2:06 | 2:12 |

TABLE 29
Formulation Robustness: HMW by HP-SEC

| | Time Point (Months, M); HMW (%) | | | | | | | | | |
|---|---|---|---|---|---|---|---|---|---|---|
| | | 5° C. | | | | 25° C. | | | 40° C. | |
| Level | Initial | 3 M | 6 M | 12 M | 18 M | 1 M | 3 M | 6 M | 1 M | 3 M |
| Lo-Lo | 0.5 | 0.6 | 0.6 | 0.6 | 0.7 | 0.6 | 0.7 | 0.8 | 0.9 | 1.2 |
| Lo-Hi | 0.5 | 0.5 | 0.6 | 0.5 | 0.7 | 0.6 | 0.6 | 0.7 | 0.7 | 0.9 |
| Hi-Lo | 0.6 | 0.6 | 0.7 | 0.7 | 0.8 | 0.7 | 0.8 | 0.9 | 1.1 | 1.6 |
| Hi-Hi | 0.5 | 0.6 | 0.6 | 0.5 | 0.7 | 0.6 | 0.6 | 0.7 | 0.8 | 1.1 |
| Target | 0.5 | 0.5 | 0.6 | 0.5 | 0.7 | 0.6 | 0.6 | 0.7 | 0.8 | 1.0 |

TABLE 30
Formulation Robustness: Monomer by HP-SEC

| | Time Point (Months, M); Monomer (%) | | | | | | | | | |
|---|---|---|---|---|---|---|---|---|---|---|
| | | 5° C. | | | | 25° C. | | | 40° C. | |
| Level | Initial | 3 M | 6 M | 12 M | 18 M | 1 M | 3 M | 6 M | 1 M | 3 M |
| Lo-Lo | 97.9 | 97.5 | 97.6 | 97.5 | 97.9 | 97.1 | 97.0 | 97.4 | 97.0 | 96.4 |
| Lo-Hi | 98.2 | 97.3 | 97.7 | 98.1 | 97.9 | 97.2 | 97.2 | 97.5 | 97.2 | 96.7 |
| Hi-Lo | 98.1 | 97.3 | 97.5 | 96.7 | 97.7 | 97.1 | 97.0 | 97.3 | 96.7 | 95.9 |
| Hi-Hi | 98.1 | 97.3 | 97.6 | 96.7 | 97.7 | 97.3 | 97.1 | 97.5 | 97.1 | 96.3 |
| Target | 98.1 | 97.3 | 97.6 | 96.8 | 97.8 | 97.5 | 97.1 | 97.5 | 97.2 | 96.6 |

TABLE 31
Formulation Robustness: LMW by HP-SEC

| | Time Point (Months, M); LMW (%) | | | | | | | | | |
|---|---|---|---|---|---|---|---|---|---|---|
| | | 5° C. | | | | 25° C. | | | 40° C. | |
| Level | Initial | 3 M | 6 M | 12 M | 18 M | 1 M | 3 M | 6 M | 1 M | 3 M |
| Lo-Lo | 1.6 | 2.0 | 1.8 | 2.0 | 1.4 | 2.3 | 2.4 | 1.8 | 2.1 | 2.4 |
| Lo-Hi | 1.3 | 2.2 | 1.8 | 1.3 | 1.5 | 2.2 | 2.2 | 1.8 | 2.0 | 2.4 |
| Hi-Lo | 1.4 | 2.1 | 1.8 | 2.6 | 1.4 | 2.2 | 2.2 | 1.8 | 2.2 | 2.5 |
| Hi-Hi | 1.4 | 2.2 | 1.8 | 2.8 | 1.6 | 2.1 | 2.3 | 1.8 | 2.0 | 2.6 |
| Target | 1.4 | 2.2 | 1.9 | 2.7 | 1.5 | 2.0 | 2.3 | 1.8 | 2.0 | 2.4 |

TABLE 32
Formulation Robustness: Minor Peak and Major Peak by CE-SDS NR

| | Time Point (Months, M); Minor Peak (%) | | | | | Time Point (Months, M); Major Peak (%) | | | | |
|---|---|---|---|---|---|---|---|---|---|---|
| | | 5° C. | 25° C. | 40° C. | | | 5° C. | 25° C. | 40° C. | |
| Level | Initial | 18 M | 3 M | 1 M | 3 M | Initial | 18 M | 3 M | 1 M | 3 M |
| Lo-Lo | 1.5 | 1.4 | 1.0 | 1.9 | 1.6 | 98.5 | 98.6 | 99.0 | 98.1 | 98.4 |
| Lo-Hi | 2.0 | 1.3 | 1.8 | 2.1 | 1.2 | 98.0 | 98.8 | 98.2 | 97.9 | 98.8 |
| Hi-Lo | 1.9 | 1.3 | 1.9 | 1.6 | 2.3 | 98.1 | 98.8 | 98.1 | 98.4 | 97.7 |
| Hi-Hi | 2.1 | 1.2 | 1.5 | 2.1 | 2.3 | 97.9 | 98.8 | 98.5 | 97.9 | 97.7 |
| Target | 2.1 | 1.2 | 1.9 | 1.9 | 2.6 | 97.9 | 98.7 | 98.1 | 98.1 | 97.4 |

TABLE 33
Formulation Robustness: Moisture by KF

| | Time Point (Months, M); Moisture (%) w/w | | | | | | | |
|---|---|---|---|---|---|---|---|---|
| | | 5° C. | | | | 25° C. | | 40° C. |
| Level | Initial | 3 M | 6 M | 12 M | 18 M | 3 M | 6 M | 3 M |
| Lo-Lo | 0.9 | 0.6 | 0.8 | 0.9 | 0.8 | 0.8 | 0.9 | 0.9 |
| Lo-Hi | 0.9 | 0.8 | 0.9 | 0.9 | 0.8 | 0.9 | 0.8 | 1.0 |
| Hi-Lo | 0.7 | 0.5 | 0.7 | 0.9 | 0.7 | 0.7 | 0.8 | 0.9 |
| Hi-Hi | 0.5 | 0.7 | 0.8 | 0.9 | 0.9 | 0.8 | 0.9 | 0.9 |
| Target | 0.8 | 0.8 | 0.8 | 0.9 | 0.8 | 0.8 | 0.9 | 0.9 |

TABLE 34
Formulation Robustness: Acidic Species by Desialylated iCIEF

| | Time Point (Months, M); Acidic Species (%) | | | | | | | | | |
|---|---|---|---|---|---|---|---|---|---|---|
| | | 5° C. | | | | 25° C. | | | 40° C. | |
| Level | Initial | 3 M | 6 M | 12 M | 18 M | 1 M | 3 M | 6 M | 1 M | 3 M |
| Lo-Lo | 22.5 | 20.0 | 29.1 | 25.2 | 21.2 | 22.6 | 22.5 | 23.5 | 21.3 | 19.7 |
| Lo-Hi | 21.9 | 22.8 | 26.8 | 24.2 | 20.6 | 22.0 | 22.2 | 24.7 | 22.1 | 19.6 |
| Hi-Lo | 23.6 | 21.8 | 26.9 | 26.3 | 21.5 | 25.4 | 23.9 | 28.2 | 26.2 | 19.9 |
| Hi-Hi | 24.5 | 22.7 | 27.1 | 27.1 | 21.6 | 22.4 | 36.7 | 27.7 | 24.0 | 20.7 |
| Target | 26.6 | 22.3 | 27.0 | 23.2 | 20.6 | 21.8 | 20.1 | 27.4 | 24.2 | 20.4 |

TABLE 35
Formulation Robustness: Total Main Species by Desialylated iCIEF

| | Time Point (Months, M); Total Main Species (%) | | | | | | | | | |
|---|---|---|---|---|---|---|---|---|---|---|
| | | 5° C. | | | | 25° C. | | | 40° C. | |
| Level | Initial | 3 M | 6 M | 12 M | 18 M | 1 M | 3 M | 6 M | 1 M | 3 M |
| Lo-Lo | 63.2 | 65.1 | 57.1 | 60.4 | 69.4 | 62.6 | 62.2 | 66.3 | 62.7 | 63.5 |
| Lo-Hi | 63.9 | 62.3 | 59.0 | 61.5 | 69.9 | 62.9 | 62.7 | 60.6 | 62.1 | 63.3 |
| Hi-Lo | 61.7 | 63.0 | 58.3 | 56.8 | 68.7 | 59.2 | 61.2 | 57.4 | 57.8 | 61.8 |
| Hi-Hi | 61.6 | 62.2 | 59.0 | 59.2 | 69.6 | 63.0 | 51.2 | 57.6 | 60.5 | 62.1 |
| Target | 59.1 | 62.9 | 58.8 | 61.8 | 69.6 | 63.3 | 64.5 | 57.8 | 60.2 | 62.2 |

TABLE 36
Formulation Robustness: Basic Species by Desialylated iCIEF

| | Time Point (Months, M); Basic Species (%) | | | | | | | | | |
|---|---|---|---|---|---|---|---|---|---|---|
| | | 5° C. | | | | 25° C. | | | 40° C. | |
| Level | Initial | 3 M | 6 M | 12 M | 18 M | 1 M | 3 M | 6 M | 1 M | 3 M |
| Lo-Lo | 14.3 | 15.0 | 13.8 | 14.5 | 9.4 | 14.8 | 15.3 | 10.2 | 16.0 | 17.9 |
| Lo-Hi | 14.3 | 14.9 | 14.2 | 14.3 | 9.5 | 15.2 | 15.1 | 14.8 | 15.8 | 17.1 |
| Hi-Lo | 14.7 | 15.3 | 14.7 | 16.9 | 9.7 | 15.4 | 15.0 | 14.4 | 16.0 | 18.3 |
| Hi-Hi | 13.9 | 15.1 | 14.0 | 13.7 | 8.8 | 14.6 | 12.0 | 14.7 | 15.5 | 17.2 |
| Target | 14.3 | 14.9 | 14.3 | 15.0 | 9.8 | 14.8 | 15.4 | 14.7 | 15.6 | 17.5 |

TABLE 37
Formulation Robustness: ≥2 μm and ≥5 μm Particulate Matter Analysis by HIAC

| | Time Point (Months, M): ≥2 μm Particulates per mL | | | | | | | | |
|---|---|---|---|---|---|---|---|---|---|
| | | 5° C. | | | 25° C. | | | 40° C. | |
| Level | Initial | 3 M | 6 M | 12 M | 1 M | 3 M | 6 M | 1 M | 3 M |
| Lo-Lo | 2,260 | 3,365 | 360 | 5,305 | 2,373 | 855 | 428 | 980 | 773 |
| Lo-Hi | 1,013 | 773 | 1,573 | 3,675 | 2,000 | 925 | 1,610 | 1,533 | 665 |
| Hi-Lo | 1,205 | 2,073 | 513 | 3,095 | 1,448 | 913 | 915 | 1,818 | 1,408 |
| Hi-Hi | 1,058 | 828 | 283 | 3,968 | 1,010 | 1,938 | 638 | 588 | 2,245 |
| Target | 620 | 1,648 | 360 | 3,417 | 1,158 | 1,340 | 2,263 | 1,778 | 1,045 |

| | Time Point (Months, M): ≥5 μm Particulates per mL | | | | | | | | |
|---|---|---|---|---|---|---|---|---|---|
| | | 5° C. | | | 25° C. | | | 40° C. | |
| Level | Initial | 3 M | 6 M | 12 M | 1 M | 3 M | 6 M | 1 M | 3 M |
| Lo-Lo | 415 | 430 | 100 | 1,162 | 1,738 | 160 | 138 | 155 | 240 |
| Lo-Hi | 143 | 75 | 660 | 888 | 1,335 | 153 | 438 | 710 | 123 |
| Hi-Lo | 350 | 775 | 38 | 883 | 610 | 233 | 230 | 420 | 425 |
| Hi-Hi | 205 | 260 | 40 | 1,142 | 370 | 768 | 155 | 150 | 365 |
| Target | 175 | 363 | 48 | 817 | 445 | 248 | 823 | 953 | 193 |

TABLE 38
Formulation Robustness: ≥10 μm and ≥25 μm Particulate

| | Time Point (Months, M): ≥10 μm | | | | | | | | |
|---|---|---|---|---|---|---|---|---|---|
| | Particulates per mL | | | | | | | | |
| | Ini- | 5° C. | | | 25° C. | | | 40° C. | |
| Level | tial | 3 M | 6 M | 12 M | 1 M | 3 M | 6 M | 1 M | 3 M |
| Lo-Lo | 143 | 58 | 15 | 445 | 863 | 63 | 80 | 45 | 55 |
| Lo-Hi | 28 | 10 | 80 | 345 | 533 | 20 | 90 | 113 | 13 |
| Hi-Lo | 65 | 245 | 3 | 353 | 168 | 43 | 50 | 105 | 98 |
| Hi-Hi | 30 | 53 | 3 | 433 | 105 | 393 | 28 | 30 | 55 |
| Target | 58 | 80 | 5 | 190 | 88 | 58 | 43 | 393 | 40 |
| | Time Point (Months, M): ≥25 μm | | | | | | | | |
| | Particulates per mL | | | | | | | | |
| | Ini- | 5° C. | | | 25° C. | | | 40° C. | |
| Level | tial | 3 M | 6 M | 12 M | 1 M | 3 M | 6 M | 1 M | 3 M |
| Lo-Lo | 30 | 13 | 0 | 32 | 20 | 3 | 8 | 18 | 3 |
| Lo-Hi | 3 | 0 | 3 | 32 | 10 | 0 | 3 | 5 | 3 |
| Hi-Lo | 3 | 0 | 0 | 25 | 0 | 0 | 3 | 5 | 5 |
| Hi-Hi | 0 | 3 | 0 | 30 | 0 | 218 | 0 | 3 | 0 |
| Target | 3 | 5 | 3 | 12 | 5 | 10 | 5 | 8 | 0 |

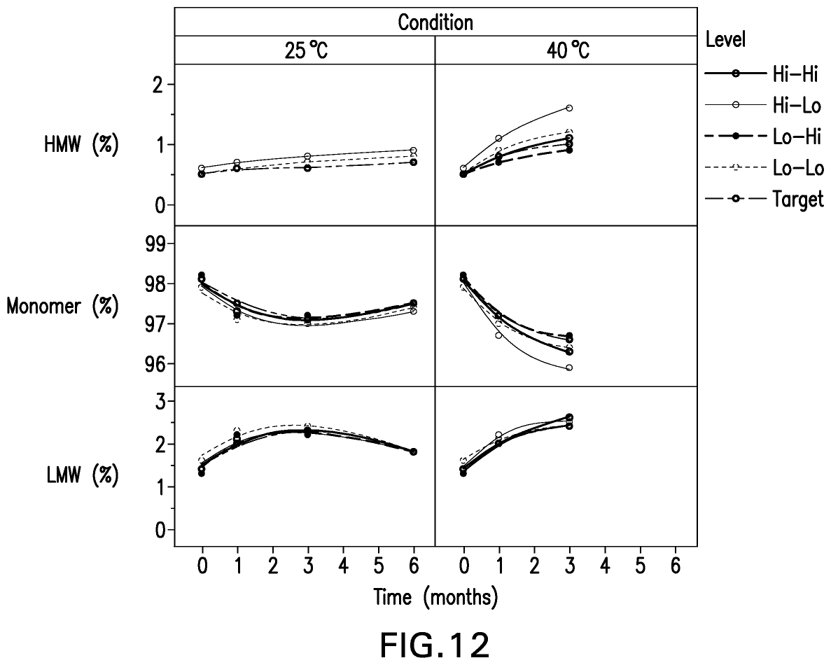

There was no discernible trend in the reconstitution time, pH or moisture content of the prototypes screened in this study at any of the tested conditions. There was no significant difference in the stability profiles of formulation prototypes for HMW, monomer, or LMW content for 5° C. and 25° C. storage temperatures and this is aligned with fragmentation pattern seen by CE-SDS non reduced. There was a slight differentiation between the formulations in 45° C. storage with the high protein concentration (55 mg/mL) and low sucrose concentration (7%) formulation, Hi-Lo, having slightly more HMW (˜0.5%) species than the others on average as shown in FIG. 12.

There was no significant difference in the stability profiles of each formulation for acidic species, main species, or basic species content at each of the storage temperatures. There was some expected variation in the various charge species, where acidic species varied between 20-30%, total main species between 55-70%, and basic species between 10-20%.

There were no discernible trends in the particulate matter/mL monitored by HIAC for any of the formulation prototypes at sizes ≥10 & ≥25 μm and were within the USP<787> specified acceptance criteria under all conditions, as the nominal fill volume for the 60 mg/Vial is 1.45 mL. In conclusion, this study demonstrates the robust nature of the formulation composition within the potential compositional variability i.e., ±5 mg/mL of the target protein concentration of 50 mg/mL and within ±1% of the target sucrose concentration of 8% (weight/volume).

TABLE 1
Sequences
Sequences

| | |
|---|---|
| SEQ ID NO: 9 | MGAAAKLAFA VFLISCSSGA ILGRSETQEC LFFNANWEKD |
| | RTNQTGVEPC YGDKDKRRHC FATWKNISGS IEIVKQGCWL |
| | DDINCYDRTD CVEKKDSPEV YFCCCEGNMC NEKFSYFPEM |
| | EVTQPTSNPV TPKPPYYNIL LYSLVPLMLI AGIVICAFWV |
| | YRHHKMAYPP VLVPTQDPGP PPPSPLLGLK PLQLLEVKAR |
| | GRFGCVWKAQ LLNEYVAVKI FPIQDKQSWQ NEYEVYSLPG |
| | MKHENILQFI GAEKRGTSVD VDLWLITAFH EKGSLSDFLK |
| | ANVVSWNELC HIAETMARGL AYLHEDIPGL KDGHKPAISH |
| | RDIKSKNVLL KNNLTACIAD FGLALKFEAG KSAGDTHGQV |
| | GTRRYMAPEV LEGAINFORD AFLRIDMYAM GLVLWELASR |
| | CTAADGPVDE YMLPFEEEIG QHPSLEDMQE VVVHKKKRPV |
| | LRDYWQKHAG MAMLCETIEE CWDHDAEARL SAGCVGERIT |
| | QMQRLTNIIT TEDIVTVVTM VTNVDFPPKE SSL |
| SEQ ID NO: 10 | ILGRSETQECLFFNANWEKDRTNQTGVEPCYGDKDKRRHCFAT |
| | WKNISGSIEIVKQGCWLDDINCYDRTDCVEKKDSPEVYFCCCE |
| | GNMCNEKFSYFPEMEVTQPTSNPVTPKPP |
| SEQ ID NO: 11 | ILGRSETQECLFFNANWEKDRTNQTGVEPCYGDKDKRRHCFAT |
| | WKNISGSIEIVKQGCWLDDINCYDRTDCVEKKDSPEVYFCCCE |
| | GNMCNEKFSYFPEM |
| SEQ ID NO: 12 | ATGGGAGCTG CTGCAAAGTT GGCGTTTGCC GTCTTTCTTA |
| | TCTCCTGTTC TTCAGGTGCT ATACTTGGTA GATCAGAAAC |
| | TCAGGAGTGT CTTTTCTTTA ATGCTAATTG GGAAAAAGAC |
| | AGAACCAATC AAACTGGTGT TGAACCGTGT TATGGTGACA |
| | AAGATAAACG GCGGCATTGT TTTGCTACCT GGAAGAATAT |
| | TTCTGGTTCC ATTGAAATAG TGAAACAAGG TTGTTGGCTG |
| | GATGATATCA ACTGCTATGA CAGGACTGAT TGTGTAGAAA |
| | AAAAAGACAG CCCTGAAGTA TATTTTTGTT GCTGTGAGGG |
| | CAATATGTGT AATGAAAAGT TTTCTTATTT TCCGGAGATG |
| | GAAGTCACAC AGCCCACTTC AAATCCAGTT ACACCTAAGC |
| | CACCCTATTA CAACATCCTG CTCTATTCCT TGGTGCCACT |
| | TATGTTAATT GCGGGGATTG TCATTTGTGC ATTTTGGGTG |
| | TACAGGCATC ACAAGATGGC CTACCCTCCT GTACTTGTTC |
| | CAACTCAAGA CCCAGGACCA CCCCCACCTT CTCCATTACT |
| | AGGTTTGAAA CCACTGCAGT TATTAGAAGT GAAAGCAAGG |
| | GGAAGATTTG GTTGTGTCTG GAAAGCCCAG TTGCTTAACG |
| | AATATGTGGC TGTCAAAATA TTTCCAATAC AGGACAAACA |
| | GTCATGGCAA AATGAATACG AAGTCTACAG |
| | TTTGCCTGGA ATGAAGCATG AGAACATATT ACAGTTCATT |
| | GGTGCAGAAA AACGAGGCAC CAGTGTTGAT GTGGATCTTT |
| | GGCTGATCAC AGCATTTCAT GAAAAGGGTT CACTATCAGA |
| | CTTTCTTAAG GCTAATGTGG TCTCTTGGAA TGAACTGTGT |
| | CATATTGCAG AAACCATGGC TAGAGGATTG GCATATTTAC |
| | ATGAGGATAT ACCTGGCCTA AAAGATGGCC ACAAACCTGC |
| | CATATCTCAC AGGGACATCA AAAGTAAAAA TGTGCTGTTG |
| | AAAAACAACC TGACAGCTTG CATTGCTGAC TTTGGGTTGG |
| | CCTTAAAATT TGAGGCTGGC AAGTCTGCAG GCGATACCCA |
| | TGGACAGGTT GGTACCCGGA GGTACATGGC TCCAGAGGTA |
| | TTAGAGGGTG CTATAAACTT CCAAAGGGAT GCATTTTTGA |
| | GGATAGATAT GTATGCCATG GGATTAGTCC TATGGGAACT |
| | GGCTTCTCGC TGTACTGCTG CAGATGGACC TGTAGATGAA |
| | TACATGTTGC CATTTGAGGA GGAAATTGGC CAGCATCCAT |
| | CTCTTGAAGA CATGCAGGAA GTTGTTGTGC ATAAAAAAAA |
| | GAGGCCTGTT TTAAGAGATT ATTGGCAGAA ACATGCTGGA |
| | ATGGCAATGC TCTGTGAAAC CATTGAAGAA TGTTGGGATC |
| | ACGACGCAGA AGCCAGGTTA TCAGCTGGAT GTGTAGGTGA |
| | AAGAATTACC CAGATGCAGA GACTAACAAA TATTATTACC |
| | ACAGAGGACA TTGTAACAGT GGTCACAATG GTGACAAATG |
| | TTGACTTTCC TCCCAAAGAA TCTAGTCTA |
| SEQ ID NO: 13 | ATACTTGGTA GATCAGAAAC TCAGGAGTGT CTTTTCTTTA |
| | ATGCTAATTG GGAAAAAGAC AGAACCAATC AAACTGGTGT |
| | TGAACCGTGT TATGGTGACA AAGATAAACG GCGGCATTGT |
| | TTTGCTACCT GGAAGAATAT TTCTGGTTCC ATTGAAATAG |
| | TGAAACAAGG TTGTTGGCTG GATGATATCA ACTGCTATGA |
| | CAGGACTGAT TGTGTAGAAA AAAAAGACAG CCCTGAAGTA |
| | TATTTTTGTT GCTGTGAGGG CAATATGTGT AATGAAAAGT |
| | TTTCTTATTT TCCGGAGATG GAAGTCACAC AGCCCACTTC |
| | AAATCCAGTT ACACCTAAGC CACCC |
| SEQ ID NO: 32 | ILGRSETQECLFFNANWEKDRTNQTGVEPCYGDKDKRRHCFAT |
| | WKNISGSIEIVKQGCWLDDINCYDRTDCVEKKDSPEVYFCCCE |
| | GNMCNEKFSYFPEMEVTQPTSNPVTPKPPTGGGTHTCPPCPAP |
| | ELLGGPSVFLFPPKPKDTLMISRTPEVTCVVVDVSHEDPEVKF |
| | NWYVDGVEVHNAKTKPREEQYNSTYRVVSVLTVLHQDWLNGKE |
| | YKCKVSNKALPVPIEKTISKAKGQPREPQVYTLPPSREEMTKN |
| | QVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 41 | ILGRSETQECLFFNANWEKDRTNQTGVEPCYGDKDKRRHCFAT |
| | WKNISGSIEIVKQGCWLDDINCYDRTDCVEKKDSPEVYFCCCE |
| | GNMCNEKFSYFPEMEVTQPTSNPVTPKPPTGGGTHTCPPCPAP |
| | ELLGGPSVFLFPPKPKDTLMISRTPEVTCVVVDVSHEDPEVKF |
| | NWYVDGVEVHNAKTKPREEQYNSTYRVVSVLTVLHQDWLNGKE |
| | YKCKVSNKALPVPIEKTISKAKGQPREPQVYTLPPSREEMTKN |
| | QVSLTCLVKGFYPSDIAVEWESNGQPENNYKTTPPVLDSDGSF |
| | FLYSKLTVDKSRWQQGNVFSCSVMHEALHNHYTQKSLSLSPG |
| SEQ ID NO: 19 | GGG |
| SEQ ID NO: 20 | GGGG |
| SEQ ID NO: 21 | TGGGG |
| SEQ ID NO: 22 | SGGGG |
| SEQ ID NO: 23 | TGGG |
| SEQ ID NO: 24 | SGGG |
| SEQ ID NO: 25 | GGGGS |
| SEQ ID NO: 33 | MKFLVNVALVFMVVYISYIYA |
| SEQ ID NO: 34 | MDAMKRGLCCVLLLCGAVFVSP |
| SEQ ID NO: 35 | MGAAAKLAFAVFLISCSSGA |
| SEQ ID NO: 36 | MDAMKRGLCCVLLLCGAVFVSPGAAILGRSETQECLFFNANWE |
| | KDRTNQTGVEPCYGDKDKRRHCFATWKNISGSIEIVKQGCWLD |
| | DINCYDRTDCVEKKDSPEVYFCCCEGNMCNEKFSYFPEMEVTQ |
| | PTSNPVTPKPPTGGGTHTCPPCPAPELLGGPSVFLFPPKPKDT |
| | LMISRTPEVTCVVVDVSHEDPEVKFNWYVDGVEVHNAKTKPRE |
| | EQYNSTYRVVSVLTVLHQDWLNGKEYKCKVSNKALPVPIEKTI |
| | SKAKGQPREPQVYTLPPSREEMTKNQVSLTCLVKGFYPSDIAV |
| | EWESNGQPENNYKTTPPVLDSDGSFFLYSKLTVDKSRWQQGNV |
| | FSCSVMHEALHNHYTQKSLSLSPGK |
| SEQ ID NO: 37 | ATGGATGCAATGAAGAGAGGGCTCTGCTGTGTGCTGCTGCTG |
| | TGTGGAGCAGTCTTCGTTTCGCCCGGCGCCGCTATACTTGGT |
| | AGATCAGAAACTCAGGAGTGTCTTTTTTTAATGCTAATTGGG |
| | AAAAAGACAGAACCAATCAAACTGGTGTTGAACCGTGTTAT |
| | GGTGACAAAGATAAACGGCGGCATTGTTTTGCTACCTGGAAG |
| | AATATTTCTGGTTCCATTGAATAGTGAAACAAGGTTGTTGGC |
| | TGGATGATATCAACTGCTATGACAGGACTGATTGTGTAGAAA |
| | AAAAAGACAGCCCTGAAGTATATTTCTGTTGCTGTGAGGGCA |
| | ATATGTGTAATGAAAAGTTTTCTTATTTTCCGGAGATGGAAG |
| | TCACACAGCCCACTTCAAATCCAGTTACACCTAAGCCACCCA |
| | CCGGTGGTGGAACTCACACATGCCCACCGTGCCCAGCACCTG |
| | AACTCCTGGGGGGACCGTCAGTCTTCCTCTTCCCCCCAAAAC |
| | CCAAGGACACCCTCATGATCTCCCGGACCCCTGAGGTCACAT |
| | GCGTGGTGGTGGACGTGAGCCACGAAGACCCTGAGGTCAAG |
| | TTCAACTGGTACGTGGACGGCGTGGAGGTGCATAATGCCAA |
| | GACAAAGCCGCGGGAGGAGCAGTACAACAGCACGTACCGTG |
| | TGGTCAGCGTCCTCACCGTCCTGCACCAGGACTGGCTGAATG |
| | GCAAGGAGTACAAGTGCAAGGTCTCCAACAAAGCCCTCCCA |
| | GTCCCCATCGAGAAAACCATCTCCAAAGCCAAAGGGCAGCC |
| | CCGAGAACCACAGGTGTACACCCTGCCCCCATCCCGGGAGG |
| | AGATGACCAAGAACCAGGTCAGCCTGACCTGCCTGGTCAAA |
| | GGCTTCTATCCCAGCGACATCGCCGTGGAGTGGGAGAGCAAT |
| | GGGCAGCCGGAGAACAACTACAAGACCACGCCTCCCGTGCT |
| | GGACTCCGACGGCTCCTTCTTCCTCTATAGCAAGCTCACCGT |
| | GGACAAGAGCAGGTGGCAGCAGGGGAACGTCTTCTCATGCT |
| | CCGTGATGCATGAGGCTCTGCACAACCACTACACGCAGAAG |
| | AGCCTCTCCCTGTCTCCGGGTAAATGAGAATTC |
| SEQ ID NO: 38 | ILGRSETQE |
| SEQ ID NO: 62 | ILGRSETQECIFYNANWERDRTNRTGVESCYGDKDKRRHCFAT |
| | WKNISGSIEIVKQGCWLDDINCYDRTDCIEKKDSPEVYFCCCE |
| | GNMCNERFSYFPEMEVTQPTSNPVTPKPP |
| SEQ ID NO: 65 | ILGRSETQECIYYNANWEKDKTNRSGIEPCYGDKDKRRHCFAT |
| | WKNISGSIEIVKQGCWLDDINCYDRNDCIEKKDSPEVFFCCCE |
| | GNMCNERFFYFPEMEVTQPTSNPVTPKPP |
| SEQ ID NO: 66 | ILGRSETQECIFYNANWERDRTNRTGVESCYGDKDKRRHCFAT |
| | WKNISGSIEIVKQGCWLDDINCYDRTDCIEKKDSPEVYFCCCE |
| | GNMCNERFSYFPEMEVTQPTSNPVTPKPP |
| SEQ ID NO: 67 | ILGRSETQECIYYNANWEKDKTNRSGIEPCYGDKDKRRHCFAT |
| | WKNISGSIEIVKQGCWLDDINCYDRNDCIEKKDSPEVFFCCCE |
| | GNMCNERFFYFPEMEVTQPTSNPVTPKP |
| SEQ ID NO: 68 | ILGRSETQECIFYNANWERDKTNRTGVELCYGDKDKRRHCFAT |
| | WKNISGSIEIVKQGCWLDDINCYDRTDCIEKKDSPEVYFCCCE |
| | GNMCNERFSYFPEMEVTQPTSNPVTPKP |

Preferred methods and materials are described herein, although methods and materials similar or equivalent to those described herein can also be used in the practice or testing of the presently disclosed methods and formulations. All publications, patent applications, patents, and other references mentioned herein are incorporated by reference in their entirety.

We claim:

1. A pharmaceutical formulation comprising a human ActRIIa fusion protein, a buffer, a surfactant and a stabilizer, wherein the ActRIIa fusion protein comprises the amino acid sequence of SEQ ID NO: 32 or SEQ ID NO:41.

2. The pharmaceutical formulation of claim 1, wherein the buffer is selected from the group consisting of sodium citrate, succinate, and histidine.

3. The pharmaceutical formulation of claim 1 or 2 wherein the buffer is present in an amount of between 4 mM and 50 mM.

4. The pharmaceutical formulation of claim 1 or 2 wherein the buffer is present in an amount of about 10 mM.

5. The pharmaceutical formulation of claim 1 or 2 wherein the buffer is a citrate buffer at a pH from about 5.5 to about pH 6.5.

6. The pharmaceutical formulation of claim 1 or 2 wherein the buffer is a succinate buffer at a pH from about 5.5 to about pH 6.5.

7. The pharmaceutical formulation of claim 1, or 2 wherein the buffer is a histidine buffer at a pH from about 5.5 to about pH 6.0.

8. The pharmaceutical formulation of claim 1, or 2 wherein the buffer is a citrate buffer at a pH 5.8.

9. The pharmaceutical formulation of claim 1 or 2 wherein the buffer is a succinate buffer at a pH of 5.8.

10. The pharmaceutical formulation of claim 5 or 8, wherein the citrate buffer comprises citric acid monohydrate and tri-sodium citrate dihydrate.

11. The pharmaceutical formulation of any one of claims 1-10, wherein the surfactant is polysorbate 80 or polysorbate 20.

12. The pharmaceutical formulation of claim 11, wherein the surfactant is present in an amount of from about 0.01 to about 0.05 weight/volume.

13. The pharmaceutical formulation of claim 11, wherein the surfactant is present in an amount of about 0.02% weight/volume.

14. The pharmaceutical formulation of any one of claims 1-13, wherein the stabilizer is sucrose.

15. The pharmaceutical formulation of claim 14 wherein sucrose is present in an amount of about 8% to about 10% weight/volume.

16. The pharmaceutical formulation of claim 14 wherein sucrose is present in an amount of about 8% weight/volume.

17. The pharmaceutical formulation of any one of claims 1-16, wherein the human ActRIIa fusion protein is present in an amount of about 75 mg/mL or less.

18. The pharmaceutical formulation of any one of claims 1-16, wherein the human ActRIIa fusion protein is present in an amount of about 50 mg/mL.

19. The pharmaceutical formulation of any one of claims 1-18, wherein the pharmaceutical formulation does not contain a salt.

20. The pharmaceutical formulation of any one of claims 1-19, wherein the ActRIIa fusion protein is sotatercept.

21. The pharmaceutical formulation of any one of claims 1-19, wherein the ActRIIa fusion protein comprises the amino acid sequence of SEQ ID NO: 32.

22. The pharmaceutical formulation of any one of claims 1-19, wherein the ActRIIa fusion protein comprises the amino acid sequence of SEQ ID NO: 41.

23. The pharmaceutical formulation of any one of claims 1-19, wherein the formulation comprises a first ActRIIa fusion protein comprising the amino acid sequence of SEQ ID NO: 32 and a second ActRIIa fusion protein comprising the amino acid sequence of SEQ ID NO: 41.

24. A lyophilized pharmaceutical formulation made by lyophilizing the formulation of any one of claims 1-23.

25. The lyophilized pharmaceutical formulation of claim 24, wherein the human ActRIIa fusion protein is provided in an amount of 45 mg/vial or 60 mg/vial.

26. A reconstituted pharmaceutical formulation made by reconstituting the lyophilized pharmaceutical formulation of claim 24.

27. The reconstituted pharmaceutical formulation of claim 26, wherein the lyophilized pharmaceutical formulation is reconstituted with Sterile Water for Injection.

28. The reconstituted pharmaceutical formulation of claim 26, wherein the lyophilized formulation is reconstituted with Sterile Water for Injection to a final protein concentration of approximately 45 mg/mL to 55 mg/mL.

29. The reconstituted pharmaceutical formulation of claim 28, wherein the lyophilized formulation is reconstituted with Sterile Water for Injection to a final protein concentration of approximately 50 mg/mL.

30. The reconstituted pharmaceutical formulation of claim 26, wherein the lyophilized formulation is reconstituted with approximately 0.5 mL to 1.8 mL of Sterile Water for Injection.

31. The reconstituted pharmaceutical formulation of claim 26, wherein the lyophilized formulation is provided in an amount of 45 mg/vial, and wherein the formulation is reconstituted with between 0.65 mL to 0.75 mL of Sterile Water for Injection.

32. The reconstituted pharmaceutical formulation of claim 26, wherein the lyophilized formulation is provided in an amount of 60 mg/vial, and wherein the formulation is reconstituted with between 0.65 mL to 1.75 mL of Sterile Water for Injection.

33. A lyophilized pharmaceutical formulation comprising a human ActRIIa fusion protein, a buffer, a surfactant and a stabilizer, wherein the buffer is selected to be physiologically compatible and to maintain a pH of 5.8 when reconstituted with Sterile Water for Injection and wherein the human ActRIIa fusion protein comprises the amino acid sequence of SEQ ID NO:32 or SEQ ID NO:41.

34. The lyophilized pharmaceutical formulation of claim 33, wherein the buffer comprises citrate.

35. The lyophilized pharmaceutical formulation of claim 33 or 34, wherein the stabilizer is sucrose.

36. The lyophilized pharmaceutical formulation of any one of claims 33-35, wherein the surfactant is polysorbate 20 or polysorbate 80.

37. The lyophilized pharmaceutical formulation of claim 36, wherein the surfactant is polysorbate 80.

38. A lyophilized pharmaceutical formulation comprising a human ActRIIa fusion protein, citrate, polysorbate 80, and sucrose, wherein the ActRIIa fusion protein comprises the amino acid sequence of SEQ ID NO:32 or SEQ ID NO:41.

39. A lyophilized pharmaceutical formulation comprising 55.0 mg of a human ActRIIa fusion protein, 0.48 mg of citric acid monohydrate, 2.56 mg tri-sodium citrate dihydrate, 0.22 mg polysorbate 80, and 88.0 mg sucrose; wherein the human ActRIIa fusion protein comprises the amino acid sequence of SEQ ID NO:32 or SEQ ID NO:41.

40. A lyophilized pharmaceutical formulation comprising 72.5 mg of a human ActRIIa fusion protein, 0.64 mg of citric acid monohydrate, 3.37 mg tri-sodium citrate dihydrate, 0.29 mg polysorbate 80, and 116.0 mg sucrose; wherein the human ActRIIa fusion protein comprises the amino acid sequence of SEQ ID NO:32 or SEQ ID NO:41.

41. A reconstituted pharmaceutical formulation comprising 50 mg/mL of a human ActRIIa fusion protein, 10 mM citrate, 0.2 mg/ml polysorbate 80, and 80 mg/ml sucrose at pH 5.8, wherein the human ActRIIa fusion protein comprises the amino acid sequence of SEQ ID NO:32 or SEQ ID NO:41.

42. The pharmaceutical formulation any one of claims 1-41, wherein the pharmaceutical formulation is administered via subcutaneous injection.

43. The pharmaceutical formulation of any one of claims 1-41, wherein the formulation is contained in a glass vial or injection device.

44. A method of treating pulmonary arterial hypertension (PAH) in a human patient in need thereof, comprising administering to the patient a pharmaceutical formulation of any one of claims 1-43.

45. A method of treating pulmonary arterial hypertension (PAH) in a human patient in need thereof, comprising reconstituting the lyophilized pharmaceutical formulation of any one of claim 24, 25, or 33-40 to create a reconstituted formulation and administering the reconstituted formulation to the patient.

46. Use of the pharmaceutical formulation of any one of claims 1-43, for the treatment of pulmonary arterial hypertension (PAH) in a patient in need thereof.


SEQUENCE LISTING (68 sequences)

SEQ ID NO: 1-8 - skipped (8 entries)

SEQ ID NO: 9 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 10 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 11 - protein, 100 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 12 - DNA, 1539 bp, Homo sapiens
  source 1..1539: mol_type = genomic DNA
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 13 - DNA, 345 bp, Homo sapiens
  source 1..345: mol_type = genomic DNA
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 14-19 - skipped (6 entries)

SEQ ID NO: 20 - protein, 4 aa, synthetic construct
GGGG

SEQ ID NO: 21 - protein, 5 aa, synthetic construct
TGGGG

SEQ ID NO: 22 - protein, 5 aa, synthetic construct
SGGGG

SEQ ID NO: 23 - protein, 4 aa, synthetic construct
TGGG

SEQ ID NO: 24 - protein, 4 aa, synthetic construct
SGGG

SEQ ID NO: 25 - protein, 5 aa, synthetic construct
GGGGS

SEQ ID NO: 26-31 - skipped (6 entries)

SEQ ID NO: 32 - protein, 344 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 21 aa, synthetic construct
MKFLVNVALVFMVVYISYIYA

SEQ ID NO: 34 - protein, 22 aa, synthetic construct
MDAMKRGLCCVLLLCGAVFVSP

SEQ ID NO: 35 - protein, 20 aa, synthetic construct
MGAAAKLAFAVFLISCSSGA

SEQ ID NO: 36 - protein, 369 aa, synthetic construct
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1114 bp, synthetic construct
  source 1..1114: mol_type = other DNA
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 38 - protein, 9 aa, synthetic construct
ILGRSETQE

SEQ ID NO: 39-40 - skipped (2 entries)

SEQ ID NO: 41 - protein, 343 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42-61 - skipped (20 entries)

SEQ ID NO: 62 - protein, 115 aa, Ovis aries
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 63 - protein, 115 aa, unidentified
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKAP

SEQ ID NO: 64 - protein, 115 aa, Mus sp.
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPP

SEQ ID NO: 65 - protein, 115 aa, Gallus gallus
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 66 - protein, 115 aa, Bos taurus; identical to SEQ ID NO: 62

SEQ ID NO: 67 - protein, 115 aa, Tyto alba; identical to SEQ ID NO: 65

SEQ ID NO: 68 - protein, 115 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKPP